(19) United States
(12) Patent Application Publication
(10) Pub. No.: US 2024/0343818 A1
(43) Pub. Date: Oct. 17, 2024

(54) Antibodies Having Humanized Framework Regions

(71) Applicant: R.P. Scherer Technologies, LLC, Carson City, NV (US)

(72) Inventors: Stepan Chuprakov, Walnut Creek, CA (US); Ayodele O. Ogunkoya, Berkeley, CA (US); Penelope M. Drake, Castro Valley, CA (US); Yun Kim, Walnut Creek, CA (US); Maxine Bauzon, Hercules, CA (US); Colin Hickle, Martinez, CA (US); Robyn M. Barfield, Emeryville, CA (US)

(21) Appl. No.: 18/578,669

(22) PCT Filed: Aug. 24, 2022
(86) PCT No.: PCT/US2022/075424

§ 371 Date: Jan. 12, 2024

Related U.S. Application Data
(60) Provisional application No. 63/236,928, filed on Aug. 25, 2021.

(51) Int. Cl. C07K 16/28; A61K 47/54; A61K 47/68; A61P 35/00

(52) U.S. Cl. CPC C07K 16/2878; A61K 47/549; A61K 47/68031; A61K 47/68033; A61K 47/6849; A61P 35/00; C07K 2317/24; C07K 2317/31; C07K 2317/565; C07K 2317/567

(57) ABSTRACT

The present disclosure provides binding agents, particularly, antibodies, that comprise variable regions comprising humanized framework regions. Nucleic acids that encode one or both of the variable chains of the binding agents of the present disclosure are also provided, as are cells that include such nucleic acids. Also provided are compositions, including in some instances, pharmaceutical compositions, that include the binding agents disclosed herein. Methods of making and using the binding agents of the present disclosure are also provided. In certain aspects, provided are methods that include administering to an individual having a cell proliferative disorder a therapeutically effective amount of a binding agent disclosed herein, where the binding agent is administered to the individual to enhance an immune response, e.g., a T cell response, to abnormally proliferating cells. The binding agents are also useful in various diagnostic, and monitoring applications, which are also provided.

### CROSS-REFERENCE TO RELATED APPLICATIONS

This application claims the benefit of priority to U.S. Provisional Application No. 63/236,928, filed Aug. 25, 2021, the disclosure of which is incorporated herein by reference.

### INCORPORATION-BY-REFERENCE OF MATERIAL ELECTRONICALLY SUBMITTED

A Sequence Listing is provided herewith as a Sequence Listing XML, “RDWD-043WO_SEQ_LIST” created on Aug. 24, 2022 and having a size of 150 KB. The contents of the Sequence Listing XML are incorporated by reference herein in their entirety.

### INTRODUCTION

Antibody biologics are becoming more clinically prevalent and, thus, present a promising class of drugs for treating several diseases, with cancer being a particularly important target for treatment with antibodies. For example, an antibody against CD30, namely brentuximab, is used for the treatment of adult patients with previously untreated stage III or IV classical Hodgkin lymphoma (cHL) in combination with chemotherapy.

To treat a chronic disease, such as cancer, it is typically necessary to repeatedly administer a biological, such as an antibody on over months or years. However, a patient's immune system may generate its own antibodies directed against the administered antibodies, thereby inducing undesirable immune response, decrease efficacy of the antibody drugs, and present clinical complications.

Therefore, therapeutics, such as antibodies are desired that do not induce an immune response when administered to humans.

### SUMMARY

The present disclosure provides binding agents comprising humanized amino acid sequences, particularly, in the framework regions of the antigen binding portions of the binding agents. In some embodiments, the present disclosure provides antibodies that specifically bind to a target, such as CD30, the antibodies comprising humanized amino acid sequences, particularly, in the framework regions of the variable heavy (VH) and variable light (VL) chains of the binding agents, such as antibodies.

Certain embodiments of the disclosure provide a binding agent that specifically binds to an antigen, the binding agent comprising:
a VH chain comprising H-CDR1, H-CDR2, and H-CDR3 and a VL chain comprising L-CDR1, L-CDR2, and L-CDR3, wherein the CDRs determine the binding specificity of the binding agent for the antigen, and wherein, in the binding agent:
the VH chain comprises:
i) a heavy chain framework region 1 (HFR1) having the sequence of SEQ ID NO: 7, a heavy chain framework region 2 (HFR2) having the sequence of SEQ ID NO: 8, a heavy chain framework region 3 (HFR3) having the sequence of SEQ ID NO: 9, and a heavy chain framework region 4 (HFR4) having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and the VL chain comprises:
i) a light chain framework region 1 (LFR1) having the sequence of SEQ ID NO: 22, a light chain framework region 2 (LFR2) having the sequence of SEQ ID NO: 23, a light chain framework region 3 (LFR3) having the sequence of SEQ ID NO: 24, and a light chain framework region 4 (LFR4) having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 25.

In some cases, the binding agent specifically binds to CD30, and comprises: a VH chain comprising H-CDR1, H-CDR2, and H-CDR3 having the sequences of SEQ ID NOs: 31-33, respectively; and VL chain comprising L-CDR1, L-CDR2, and L-CDR3 having the sequences of SEQ ID NOs: 34-36, respectively.

In certain such embodiments, the binding agent comprises i) a VH chain comprising a sequence selected from: SEQ ID NO: 6 and SEQ ID NO: 11; and ii) a VL chain comprising a sequence selected from: SEQ ID NO: 21 and SEQ ID NO: 26.

The binding agents can be an antibody, such as IgG, Fv, single chain antibody, scFv, Fab, F(ab′)2, or Fab′. The binding agent can also be a T-cell receptor or T-cell receptor (TCR)-like antibody.

The binding agent can be conjugated to another moiety, such as detectable label, non-peptide synthetic polymer, lipid or fatty acid, contrast agent, affinity domain, cytotoxin, a drug, oligonucleotide, protein, lipid nanoparticle, viral particle, a water-soluble polymer, or a synthetic peptide.

Further embodiments of the disclosure provide nucleic acid encoding binding agents comprising VH chain and/or VL chains disclosed herein. The nucleic acids can be in transiently or permanently present in a host cell. For example, in a host cell, the nucleic acid encoding the binding agent comprising VH chain and/or VL chains can be operably linked to a transcriptional control element that is active in the host cell.

Even further embodiments of the disclosure provide pharmaceutical compositions comprising a binding agent disclosed herein and a pharmaceutically acceptable carrier.

Certain embodiments of the disclosure provide treating a diseases, such as a cell proliferative disorder in a subject, the method comprising: administering to the subject a therapeutically effective amount of a pharmaceutical composition comprising a binding agent disclosed herein.

### BRIEF DESCRIPTION OF THE FIGURES

FIGS. 1A-1D. (A) Sequence alignment of the heavy chain framework regions 1-4 of AC10 antibody and H1 variant disclosed herein. (B) Sequence alignment of the heavy chain framework regions 1-4 of AC10 antibody and H4 variant disclosed herein. (C) Sequence alignment of the light chain framework regions 1-4 of AC10 antibody and L2 variant disclosed herein. (B) Sequence alignment of the light chain framework regions 1-4 of AC10 antibody and L4 variant disclosed herein.

FIGS. 2A-2C. Aldehyde-tagged antibody production and ADC generation using HIPS-mediated conjugation. (A) The formylglycine recognition sequence (CXPXR) is genetically encoded into the antibody. (B) Co-translationally formylglycine-generating enzyme converts the cysteine within the recognition sequence to a formylglycine residue containing an aldehyde functional group that can be specifically conjugated with (C) the Hydrazino-iso-Pictet-Spengler (HIPS) conjugation element.

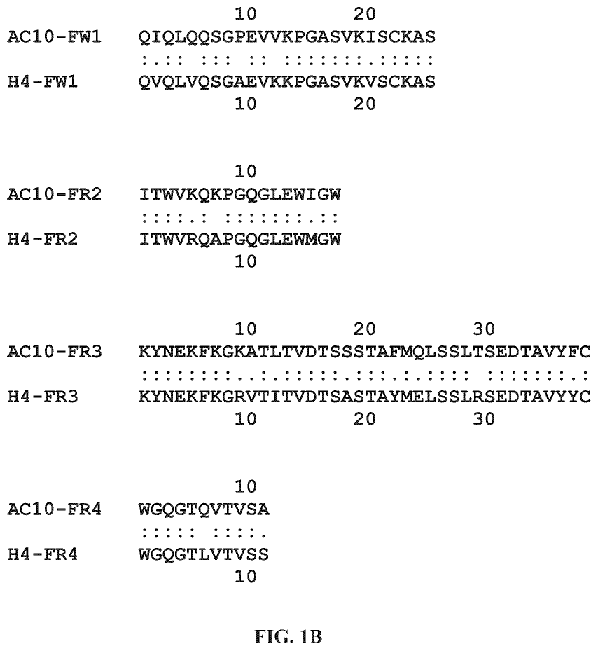

FIG. 3. CT-tagged H1/L1 antibody conjugated to a non-cleavable linker bearing a maytansine payload (RED-106) yields a DAR of 1.88 as determined by hydrophobic interaction chromatography (HIC).

FIG. 4. CT-tagged H1/L1 antibody conjugated to RED-106 is 99.2% monomeric as determined by size-exclusion chromatography (SEC).

FIG. 5. CT-tagged H1/L4 antibody conjugated to RED-106 yields a DAR of 1.91 as determined by HIC.

FIG. 6. CT-tagged H1/L4 antibody conjugated to RED-106 is 99.5% monomeric as determined by SEC.

FIG. 7. CT-tagged H4/L2 antibody conjugated to RED-106 yields a DAR of 1.89 as determined by HIC.

FIG. 8. CT-tagged H4/L2 antibody conjugated to RED-106 is 99.7% monomeric as determined by SEC.

FIG. 9. CT-tagged H4/L4 antibody conjugated to RED-106 yields a DAR of 1.90 as determined by HIC.

FIG. 10. CT-tagged H4/L4 antibody conjugated to RED-106 is 99.5% monomeric as determined by SEC.

FIG. 11. ELISA binding of humanized anti-CD30 antibodies to recombinant human CD30 protein. Antibody variants comprising various combinations of H1 heavy chain variant with L1-L5 light chains were tested. The chimeric antibody (H/L) is included for reference.

FIG. 12. ELISA binding of humanized anti-CD30 antibodies to recombinant human CD30 protein. Antibody variants comprising various combinations of H4 heavy chain variant with L1-L5 light chains were tested. The chimeric antibody (H/L) is included for reference.

FIG. 13. In vitro potency against SU-DHL-1 cells of maytansine-conjugated humanized anti-CD30 ADCs comprising various combinations of H1 heavy chain variant with L1-L5 light chain variants. An ADC made with the chimeric antibody (H/L) is included for reference.

FIG. 14. In vitro potency against L540 cells of maytansine-conjugated humanized anti-CD30 ADCs comprising various combinations of H1 heavy chain variant with L1-L5 light chain variants. An ADC made with the chimeric antibody (H/L) is included for reference.

FIG. 15. In vitro potency against SU-DHL-1 cells of maytansine-conjugated humanized anti-CD30 ADCs comprising various combinations of H4 heavy chain variant with L1-L5 light chain variants. An ADC made with the chimeric antibody (H/L) is included for reference.

FIG. 16. In vitro potency against L540 cells of maytansine-conjugated humanized anti-CD30 ADCs comprising various combinations of H4 heavy chain variant with L1-L5 light chain variants. An ADC made with the chimeric antibody (H/L) is included for reference.

FIG. 17. In vivo efficacy against the HuT 102 xenograft of maytansine-conjugated humanized anti-CD30 ADCs comprising the VH1/VL4, VH4/VL2, and VH4/VL4 variants. The VH4/VL4 variant was tested both as a DAR4 and a DAR2 composition. A single 10 mg/kg dose of ADC was administered on Day 0.

FIG. 18 shows a graph of an L-82 xenograft study with a single intravenous dose of the listed anti-CD30 ADC on Day 0. VH4/VL4 Compound 8 (RED-601) uses the internal 91N tag and delivers half the payload dose as compared to Adcetris. At 50% ADC dosing (1.5 mg/kg) and equal dosing (3 mg/kg) VH4/VL4 Compound 8 was equally efficacious as compared with Adcetris, with all arms showing 8 complete responses out of 8 mice/group. The VH4/VL4 antibody alone had minimal activity.

FIG. 19 shows a graph of a Karpas 299 xenograft study with a single intravenous dose of the listed anti-CD30 ADC on Day 0. VH4/VL4 Compound 8 (RED-601) uses the internal 91N tag and delivers half the payload dose as compared to Adcetris. At 50% ADC dosing (1.5 mg/kg) and equal dosing (3 mg/kg) VH4/VL4 Compound 8 gave 5/6 and 6/6 complete responses as compared with Adcetris, which gave 6/6 complete responses though with 2-fold the payload amount compared to VH4/VL4 Compound 8. The VH4/VL4 antibody alone had minimal activity.

FIG. 20. Single-tagged CD30 VH4/VL4 antibody conjugated at 91N to Compound 8 (RED-601) yields a DAR of 1.58 as determined by PLRP.

FIG. 21. Single-tagged CD30 VH4/VL4 antibody conjugated at 91N to Compound 8 (RED-601) is 98% monomeric as determined by SEC.

### DEFINITIONS

The term “binding agent” refers to a protein comprising a variable heavy (VH) chain and variable light (VL) chain. Each VH and VL chain comprises framework (FR) regions interrupted by complementarity determining regions (CDR). Typically, a VH chain comprises sequentially arranged HFR1-HCDR1-HFR2-HCDR2-HFR3-HCDR3-HFR4. Similarly, a VL chain typically comprises sequentially arranged LFR1-LCDR1-LFR2-LCDR2-LFR3-LCDR3-LFR4. Thus, the term “framework” when used in reference to a binding agent, such as an antibody, is intended to mean the amino acid residues outside the CDRs within the variable region of the binding agent.

The terms “antibodies” and “immunoglobulin” include antibodies or immunoglobulins of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. An antibody may be monovalent or bivalent. “Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

In some embodiments, a subject binding agent is a recombinant or modified binding agent, e.g., a chimeric, humanized, deimmunized or an in vitro generated antibody. The term “recombinant” or “modified” binding agent or antibody as used herein is intended to include all binding agents that are prepared, expressed, created, or isolated by recombinant means, such as (i) antibodies expressed using a recombinant expression vector transfected into a host cell; (ii) antibodies isolated from a recombinant, combinatorial antibody library; (iii) antibodies isolated from an animal (e.g. a mouse) that is transgenic for human immunoglobulin genes; or (iv) antibodies prepared, expressed, created, or isolated by any other means that involves splicing of human immunoglobulin gene sequences to other DNA sequences. Such recombinant antibodies include humanized, CDR grafted, chimeric, deimmunized, and in vitro generated antibodies; and can optionally include constant regions derived from human germline immunoglobulin sequences.

“Fv” is the minimum antibody fragment which contains a complete antigen-recognition and -binding site. This region consists of a dimer of one heavy- and one light-chain variable domain in a non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some aspects, the Fv polypeptide further comprises a polypeptide linker between the VH and VL domains, which enables the sFv to form the desired structure for antigen binding.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites.

The term “binding” refers to a direct association between two molecules, due to, for example, covalent, electrostatic, hydrophobic, and ionic and/or hydrogen-bond interactions, including interactions such as salt bridges and water bridges. A subject anti-CD30 antibody binds specifically to an epitope within a CD30 protein, e.g., a human CD30 protein, for example, a glycosylated CD30 or a fragment thereof. Non-specific binding would refer to binding with an affinity of less than about 10⁻⁷ M, e.g., binding with an affinity of 10⁻⁶ M, 10⁻⁵ M, 10⁻⁴ M, etc.

The term “specifically binds” in the context of an antibody and an antigen means that the antibody binds to or associates with the antigen with an affinity or Kₐ (that is, an equilibrium association constant of a particular binding interaction with units of 1/M) of, for example, greater than or equal to about 10⁵ M⁻¹.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

Throughout the present disclosure, the numbering of the residues in an immunoglobulin heavy chain and in an immunoglobulin light chain is as described by Chothia et al., J. Mol. Biol. 196:901-917 (1987), expressly incorporated herein by reference. Also, the framework regions and the CDRs as referenced in this disclosure are as described by Chothia et al.

In the context of an immunoglobulin polypeptide, the term “constant region” is well understood in the art, and refers to a C-terminal region of an Ig heavy chain, or an Ig light chain. An Ig heavy chain constant region includes CH1, CH2, and CH3 domains (and CH4 domains, where the heavy chain is a or an F heavy chain). In a native Ig heavy chain, the CH1, CH2, CH3 (and, if present, CH4) domains begin immediately after (C-terminal to) the heavy chain variable (VH) region, and are each from about 100 amino acids to about 130 amino acids in length. In a native Ig light chain, the constant region begins immediately after (C-terminal to) the light chain variable (VL) region, and is about 100 amino acids to 120 amino acids in length.

An “epitope” is a site on an antigen (e.g., a site on CD30) to which an antibody binds. Epitopes can be formed both from contiguous amino acids or noncontiguous amino acids juxtaposed by folding (e.g., tertiary folding) of a protein. Epitopes formed from contiguous amino acids are typically retained on exposure to denaturing solvents whereas epitopes formed by folding are typically lost on treatment with denaturing solvents. An epitope typically includes at least 3, and more usually, at least 5 or 8-10 amino acids in a linear or spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Several commercial laboratories offer epitope mapping services. Epitopes bound by an antibody immunoreactive with a membrane associated antigen can reside on the surface of the cell (e.g. in the extracellular region of a transmembrane protein), so that such epitopes are considered cell-surface accessible, solvent accessible, and/or cell-surface exposed.

By “genetically-encodable” as used in reference to an amino acid sequence of polypeptide, peptide or protein means that the amino acid sequence is composed of amino acid residues that are capable of production by transcription and translation of a nucleic acid encoding the amino acid sequence, where transcription and/or translation may occur in a cell or in a cell-free in vitro transcription/translation system.

The term “control sequences” refers to DNA sequences that facilitate expression of an operably linked coding sequence in a particular expression system, e.g. mammalian cell, bacterial cell, cell-free synthesis, etc. The control sequences that are suitable for prokaryote systems, for example, include a promoter, optionally an operator sequence, and a ribosome binding site. Eukaryotic cell systems may utilize promoters, polyadenylation signals, and enhancers.

A nucleic acid is “operably linked” when it is placed into a functional relationship with another nucleic acid sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate the initiation of translation. Generally, “operably linked” means that the DNA sequences being linked are contiguous, and, in the case of a secretory leader, contiguous and in reading frame. Linking is accomplished by ligation or through amplification reactions. Synthetic oligonucleotide adaptors or linkers may be used for linking sequences in accordance with conventional practice.

In certain embodiments, the antibody molecules disclosed herein include a heavy chain comprising a variable heavy chain region as provided herein and a human IgG1 constant region having the amino acid sequence set forth in UniProt: P01857-1, version 1. In certain embodiments, the antibody molecules disclosed herein include a light chain comprising a variable light chain region as provided herein and a human light chain constant region. In certain embodiments, the human light chain constant region is a human kappa light chain constant region having the amino acid set forth in UniProtKB/Swiss-Prot: P01834.2. In certain embodiments, the human IgG1 heavy chain constant region present in the subject antibodies may include mutations, e.g., substitutions to modulate Fc function. For example, the LALAPG effector function mutations (L234A, L235A, and P329G) or the N297A mutation may be introduced to reduce antibody dependent cellular cytotoxicity (ADCC). The numbering of the substitutions is based on the EU numbering system. The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, MD. (1991)). The “EU index as in Kabat” refers to the residue numbering of the human IgG 1 EU antibody.

The term “chimeric binding agent” refer to binding agents whose light and heavy chain genes have been constructed, typically by genetic engineering, from variable and constant region genes belonging to different species. For example, the variable segments of the genes from a mouse may be joined to human constant segments. An example of a “chimeric binding agent” is a chimeric antibody or chimeric TCR-like antibody. Certain aspects of TCR-like antibodies are described by He et al. (2019), J Hematol Oncol.; 12(1):99.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

The term “humanized antibodies” refer to antibodies from non-human species whose protein sequences have been modified to increase their similarity to antibodies produced naturally in humans. Humanized antibodies, when administered to humans, do not induce immune response against these antibodies or induce immune response that is much weaker compared to administration of the corresponding non-human antibodies. Humanized antibodies have at least three advantages over the original non-human antibodies: the immunogenicity of the antibody is reduced (since much of the immune response occurs against the mouse Ig constant region); the human constant region allows for human effector functions to occur; and the serum half-life of the humanized antibodies in humans is significantly increased.

The binding agents disclosed herein may also include an affinity domain, including peptide sequences that can interact with a binding partner, e.g., such as one immobilized on a solid support, useful for identification or purification. Consecutive single amino acids, such as histidine, when fused to a protein, can be used for one-step purification of the fusion protein by high affinity binding to a resin column, such as nickel sepharose. Examples of affinity domains include His5 (HHHHH) (SEQ ID NO: 177), His X6 (HHHHHH) (SEQ ID NO: 178), C-myc (EQKLISEEDL) (SEQ ID NO: 179), Flag (DYKDDDDK) (SEQ ID NO: 180), StrepTag (WSHPQFEK) (SEQ ID NO: 181), hemagglutinin, e.g., HA Tag (YPYDVPDYA; SEQ ID NO: 182), glutathinone-S-transferase (GST), thioredoxin, cellulose binding domain, RYIRS (SEQ ID NO: 183), Phe-His-His-Thr (SEQ ID NO: 184), chitin binding domain, S-peptide, T7 peptide, SH2 domain, C-end RNA tag, WEAAAREACCRECCARA (SEQ ID NO: 185), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, leucine zipper sequences, and maltose binding protein.

“Native amino acid sequence” or “parent amino acid sequence” are used interchangeably herein to refer to the amino acid sequence of a polypeptide prior to modification to include a modified amino acid residue. In this disclosure, the “native amino acid sequence” or “parent amino acid sequence” refers to the sequence found in the anti-CD30 antibody AC10 as described in the United States Patent Application Publication No. 20050123536, which is incorporated herein by reference in its entirety. Particularly, the amino acid sequences of various domains in the VH and VL chains of AC10 antibody are shown in the following Table (Table 2):

TABLE 2

| Domain | SEQ ID NO |
|---|---|
| Heavy chain framework region 1 | 2 |
| Heavy chain framework region 2 | 3 |
| Heavy chain framework region 3 | 4 |
| Heavy chain framework region 4 | 10 |
| Light chain framework region 1 | 17 |
| Light chain framework region 2 | 18 |
| Light chain framework region 3 | 19 |
| Light chain framework region 4 | 20 |
| Heavy chain CDR1 | 31 |
| Heavy chain CDR2 | 32 |
| Heavy chain CDR3 | 33 |
| Light chain CDR1 | 34 |
| Light chain CDR2 | AAS |
| Light chain CDR3 | 36 |
| Entire variable heavy chain | 1 |
| Entire variable light chain | 16 |

The term “conjugated” generally refers to a chemical linkage, either covalent or non-covalent, usually covalent, that proximally associates one molecule of interest with a second molecule of interest. In some embodiments, the agent is selected from a half-life extending moiety, a labeling agent, and a drug. For half-life extension, for example, the antibodies of the present disclosure can optionally be modified to provide for improved pharmacokinetic profile (e.g., by PEGylation, hyperglycosylation, and the like). Modifications that can enhance serum half-life are of interest.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a subject binding agent, such as an antibody, for example, anti-CD30 antibody, that, when administered to a mammal or other subject for treating a disease, is sufficient to effect such treatment for the disease. The “therapeutically effective amount” will vary depending on the antibody, the disease and its severity and the age, weight, etc., of the subject to be treated.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O) substituted alkyl, NR²⁰C(O)cycloalkyl, —NR²⁰C(O) substituted cycloalkyl, —NR²⁰C(O)cycloalkenyl, —NR²⁰C(O) substituted cycloalkenyl, —NR²⁰C(O)alkenyl, —NR²⁰C(O) substituted alkenyl, —NR²⁰C(O)alkynyl, —NR²⁰C(O) substituted alkynyl, —NR²⁰C(O)aryl, —NR²⁰C(O) substituted aryl, —NR²⁰C(O)heteroaryl, —NR²⁰C(O) substituted heteroaryl, —NR²⁰C(O)heterocyclic, and —NR²⁰C(O) substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR⁵¹R⁵², wherein R⁵¹ and R⁵² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵¹ and R⁵² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁵¹C(O)NR⁵²R⁵³ where R⁵¹, R⁵², and R⁵ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR⁵¹R⁵², wherein R⁵¹ and R⁵² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R⁵¹ and R⁵² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR⁵¹SO₂R⁵², wherein R⁵¹ and R⁵² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵¹ and R⁵² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzol[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂ alkenyl, OSO₂-substituted alkenyl, OSO₂-cycloalkyl, OSO₂-substituted cycloalkyl, OSO₂ cycloalkenyl, OSO₂-substituted cylcoalkenyl, OSO₂-aryl, OSO₂-substituted aryl, OSO₂ heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸OR⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like. In certain embodiments, a polypeptide is an antibody or a binding agent, as described herein.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an antibody” includes a plurality of such antibodies and reference to “the CDR” includes reference to one or more CDRs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As noted above, “humanized antibodies” are antibodies from non-human species whose protein sequences have been modified to increase their similarity to antibody variants produced naturally in humans. Humanized antibodies, when administered to humans, do not induce immune response against these antibodies or induce immune response that is much weaker compared to administration of the corresponding non-human antibodies. Humanized antibodies have at least three advantages over the original non-human antibodies: the immunogenicity of the antibody is reduced (since much of the immune response occurs against the mouse Ig constant region); the human constant region allows for human effector functions to occur; and the serum half-life of the humanized antibodies in humans is significantly increased.

As also described above, “framework” or “framework region” when used in reference to a variable region refers to amino acid residues outside the CDRs within the variable region of a binding protein, such as an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

Certain embodiments of the disclosure provide binding agents comprising framework regions having humanized amino acid sequences. Depending upon the CDR region inserted within the humanized framework regions, the binding agents disclosed herein can specifically bind to any antigen, particularly, CD30 protein. Also provided herein are nucleic acids that encode one or both of the variable chain polypeptides of the binding agents disclosed herein. Cells that include such nucleic acids are also described. Further provided are compositions that include the binding agents, particularly, antibodies, disclosed herein, including in some instances, pharmaceutical compositions.

Methods of making and using the binding agents of the present disclosure are also provided. In certain aspects, provided are methods that include administering to an individual having a cell proliferative disorder a therapeutically effective amount of a binding agent, particularly, an antibody of the present disclosure, where the binding agent is administered to the individual to enhance an immune response, e.g., a T cell response, to abnormally proliferating cells of the cell proliferative disorder. The antibodies are useful in various diagnostic, and monitoring applications, which are also provided.

### Humanized Framework Regions

As summarized above, the present disclosure provides binding agents comprising humanized framework regions. As a “parent amino acid sequence,” the amino acid sequence of AC-10 antibody, which is an anti-CD30 antibody, is used to derive binding agents having humanized framework regions.

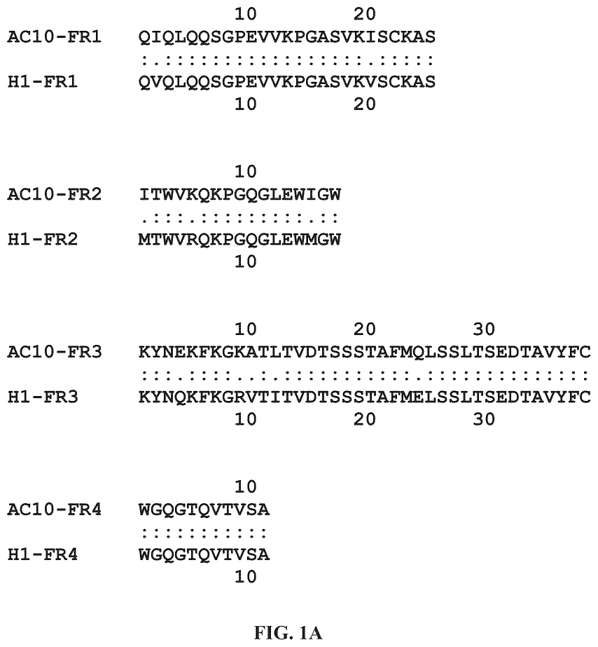

For example, FIG. 1A describes sequence alignments between the framework regions 1 to 4 of the VH chain of AC10 antibody (SEQ ID NOs: 2 to 4 and 10, respectively, for HFR1 to HFR4) with the framework regions 1 to 4 of the heavy chain variant 1 (H1 variant) (SEQ ID NOs: 7 to 10, respectively, for HFR1 to HFR4). The specific mutations in the framework regions of the H1 variant as compared to those of AC10 may render the binding agents comprising the framework regions of the H1 variant less immunogenic when administered to a human.

Similarly, FIG. 1B describes sequence alignments between the framework regions 1 to 4 of the VH chain of AC10 antibody (SEQ ID NOs: 2 to 4 and 10, respectively, for HFR1 to HFR4) with the framework regions 1 to 4 of the heavy chain variant 4 (H4 variant) (SEQ ID NOs: 12 to 15, respectively, for HFR1 to HFR4). The specific mutations in the framework regions of the H4 variant as compared to those of AC10 may render the binding agents comprising the framework regions of the H4 variant less immunogenic when administered to a human.

Further, FIG. 1C describes sequence alignments between the framework regions 1 to 4 of the VL chain of AC10 antibody (SEQ ID NOs: 17 to 20, respectively, for LFR1 to LFR4) with the framework regions 1 to 4 of the light chain variant 2 (L2 variant) (SEQ ID NOs: 22 to 25, respectively, for LFR1 to LFR4). The specific mutations in the framework regions of the L2 variant as compared to those of AC10 may render the binding agents comprising the framework regions of the L2 variant less immunogenic when administered to a human.

Further, FIG. 1D describes sequence alignment between the framework regions 1 to 4 of the VL chain of AC10 antibody (SEQ ID NOs: 17 to 20, respectively, for LFR1 to LFR4) with the framework regions 1 to 4 of the light chain variant 4 (L4 variant) (SEQ ID NOs: 27 to 30, respectively, for LFR1 to LFR4). The specific mutations in the framework regions of the L4 variant as compared to those of AC10 may render the binding agents comprising the framework regions of the L4 variant less immunogenic when administered to a human.

In certain embodiments, binding agents are described that contain:
1) the framework regions from the H1 variant with the framework regions from the L2 variant;
2) the framework regions from the H1 variant with the framework regions from the L4 variant;
3) the framework regions from the H4 variant with the framework regions from the L2 variant; or
4) the framework regions from the H4 variant with the framework regions from the L4 variant.

Certain embodiments of the disclosure provide a binding agent that comprising: a VH chain comprising H-CDR1, H-CDR2, and H-CDR3 and a VL chain comprising L-CDR1, L-CDR2, and L-CDR3, wherein the CDRs determine the binding specificity of the binding agent for the antigen, and wherein, in the binding agent:
the VH chain comprises:
i) a HFR1 having the sequence of SEQ ID NO: 7, a HFR2 having the sequence of SEQ ID NO: 8, a HFR3 having the sequence of SEQ ID NO: 9, and a HFR4 having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and
the VL chain comprises:
i) a LFR1 having the sequence of SEQ ID NO: 22, a LFR2 having the sequence of SEQ ID NO: 23, a LFR3 having the sequence of SEQ ID NO: 24, and a LFR4 having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 25.

### CDR Grafting

The term “CDR grafting” as used herein refers to grafting CDRs from an antibody into framework regions of another antibody to transfer the specificity and affinity of CDR donor antibody to the antibody providing the framework regions. While CDR grafting is typically used to transfer CDRs from non-human antibodies to framework regions of human antibodies to produce humanized antibodies, the term “CDR grafting” as used herein encompasses transferring CDRs from any donor antibody to framework regions of any acceptor antibody.

Almagro et al. (2018), Frontiers in Immunology, Vol. 8, Article 1751 (“Almagro”), describe certain aspects of CDR grafting. Particularly, Almagro teaches that CDRs from an antibody can be grafted into framework regions of another antibody to transfer the specificity and affinity of CDR donor antibody to the antibody providing the framework regions. Almagro also states that CDR grafting were used to engineer alemtuzumab, which is a humanized antibody against CD52 and approved for clinical use. CDRs from a rat IgG2a were grafted into the human VH and VL framework regions to produce alemtuzumab. Almagro also teaches CDR grafting from murine antibody was used to produce humanized antibody daclizumab. Almagro teaches that CDR grafting was used to engineer all of the humanized antibodies approved for therapeutic use as of Jul. 30, 2017. Indeed, Almagro teaches that preparing humanized antibodies by grafting rodent CDRs into human FRs constitutes a “remarkable progress in the engineering and clinical development of therapeutic antibodies.”

Thus, CDRs from a first antibody that specifically binds to an antigen can be grafted into the framework regions of a second antibody having certain beneficial characteristics attributable to the framework regions, for example, humanized sequences. The resulting antibody retains the binding specificity of the first antibody while also acquiring the beneficial characteristics of the framework regions of the second antibody, such as reduced immunogenicity in humans.

As noted above, certain embodiments of the disclosure provide antibodies having humanized framework regions. Therefore, CDRs from any antibody that specifically binds to an antigen can be grafted into the framework regions disclosed herein to produce humanized antibodies having binding specificity for the antibody that provides the CDRs.

Accordingly, certain embodiments of the disclosure provide a binding agent that specifically binds to an antigen, the binding agent comprising: a VH chain comprising H-CDR1, H-CDR2, and H-CDR3 and a VL chain comprising L-CDR1, L-CDR2, and L-CDR3, wherein the CDRs determine the binding specificity of the binding agent for the antigen, and wherein, in the binding agent:
the VH chain comprises:
i) a HFR1 having the sequence of SEQ ID NO: 7, a HFR2 having the sequence of SEQ ID NO: 8, a HFR3 having the sequence of SEQ ID NO: 9, and a HFR4 having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and the VL chain comprises:
i) a LFR1 having the sequence of SEQ ID NO: 22, a LFR2 having the sequence of SEQ ID NO: 23, a LFR3 having the sequence of SEQ ID NO: 24, and a LFR4 having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 25.

In certain embodiments, the VH chain of a binding agent comprises in the framework regions amino acid sequences having 80% or greater, 85% or greater, 90% or greater, 95% or greater, 99% or greater, or 100% sequence identity to the amino acid sequences set forth in FR1 to FR4 as described in Table 3, with the exclusion of the residues mutated in the variants as compared to the parental AC-10 sequences, i.e., the mutated residues are retained. The mutations in the H1 and H4 variants as compared to the parental AC-10 sequences are shown in FIG. 1A and FIG. 1B, respectively.

In certain embodiments, the VL chain of a CD-30 binding agent comprises in the framework regions amino acid sequences having 80% or greater, 85% or greater, 90% or greater, 95% or greater, 99% or greater, or 100% sequence identity to the amino acid sequences set forth in FR1 to FR4 as described in Table 3, with the exclusion of the residues mutated in the variants as compared to the parental AC-10 sequences, i.e., the mutated residues are retained. The mutations in the L2 and L4 variants as compared to the parental AC-10 sequences are shown in FIG. 1C and FIG. 1D, respectively.

In some cases, the binding agent is a chimeric binding agent. A chimeric binding agent can be a chimeric antibody or a chimeric TCR-like antibody.

In some cases, the binding agent is a T-cell receptor, T-cell receptor like antibody, an IgG, Fv, single chain antibody, scFv, Fab, F(ab′)₂, or Fab′. IgG can be an IgG1.

In some embodiments, a subject binding agent comprises a constant region of an immunoglobulin (e.g., an Fc region). The Fc region, if present, can be a human Fc region. If constant regions are present, the antibody can contain both light chain and heavy chain constant regions. The antibodies described herein include antibodies having all types of constant regions, including IgM, IgG, IgD, IgA and IgE, and any isotype, including IgG1, IgG2, IgG3 and IgG4. An example of a suitable heavy chain Fc region is a human isotype IgG1 Fc. Light chain constant regions can be lambda or kappa. A subject binding agent (e.g., a subject humanized antibody) can comprise sequences from more than one class or isotype. Antibodies can be expressed as tetramers containing two light and two heavy chains, as separate heavy chains, light chains, as Fab, Fab′ F(ab′)₂, and Fv, or as single chain antibodies in which heavy and light chain variable domains are linked through a spacer.

In some embodiments, a binding agent, such as an antibody, for example, an anti-CD30 antibody, disclosed herein may include one or more amino acid substitutions introduced in the Fc region. In some embodiments, the one or more of the amino acid substitutions may be at the positions 239, 298, 326, 330 and 332 in the Fc region. In some embodiments, an antibody of the present disclosure may include one or more of the following amino acid substitutions introduced in the Fc region: 1332E; S239D/A330L/I332E; S239D/S298A/I332E; S239D/K326T/I332E; S239D/S298A/K326T/I332E; or S239D/A330L/I332E/D356E/L358M.

In some embodiments, a subject binding agent, such as an antibody, comprises a free thiol (—SH) group at the carboxyl terminus, where the free thiol group can be used to attach the antibody to a second polypeptide (e.g., another antibody, including a subject antibody), a scaffold, a carrier, etc.

In some embodiments, a subject binding agent, such as an antibody comprises one or more non-naturally occurring amino acids. In some embodiments, the non-naturally encoded amino acid comprises a carbonyl group, an acetyl group, an aminooxy group, a hydrazine group, a hydrazide group, a semicarbazide group, an azide group, or an alkyne group. Inclusion of a non-naturally occurring amino acid can provide for linkage to a polymer, a second polypeptide, a scaffold, etc. Examples of such non-naturally-occurring amino acids include, but are not limited to, N-acetylglucosaminyl-L-serine, N-acetylglucosaminyl-L-threonine, and O-phosphotyrosine.

In some cases, the binding agent is a bispecific binding agent, such as a bispecific antibody. For example, a bispecific antibody can comprise two linked antigen-binding fragments or two large immunoglobulin-like molecules with additional domains attached. In such bispecific antibodies, the two variable domains can have the framework regions as described herein but have CDRs that are specific for different targets.

Full length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., PCT Intl. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T3945/Y407A, T366W/T394S, F405W/T394S and T366W/T366S/L368A/Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. U82010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351 Y/F405A/Y407V/T394W, T3661/K392M/T394W/F405A/Y407V, T366L/K392M/T394W/F405A/Y407V, L351Y/Y407A/T366A/K409F, L351Y/Y407A/T366V/K409F, Y407A/T366A/K409F, or T350V/L351Y/F405A/Y407V, T350V/T366L/K392L/T394W as described in U.S. Pat. Pub. No. US2012/0149876 or U.S. Pat. Pub. No. US2013/0195849.

Also provided are single chain bispecific antibodies. In some embodiments, a single chain bispecific antibody of the present disclosure is a bispecific scFv.

In some embodiments, a subject binding agent comprises scFv multimers. For example, in some embodiments, a subject binding agent is an scFv dimer (e.g., comprises two tandem scFv (scFv2)), an scFv trimer (e.g., comprises three tandem scFv (scFv3)), an scFv tetramer (e.g., comprises four tandem scFv (scFv4)), or is a multimer of more than four scFv (e.g., in tandem). The scFv monomers can be linked in tandem via linkers of from about 2 amino acids to about 10 amino acids in length, e.g., 2 aa, 3 aa, 4 aa, 5 aa, 6 aa, 7 aa, 8 aa, 9 aa, or 10 aa in length. Suitable linkers include, e.g., (Gly)ₓ, where x is an integer from 2 to 10 (SEQ ID NO: 191), glycine-serine polymers, and the like.

### CD30 Binding Agents

As summarized above, via CDR grafting CDRs from a first antibody that specifically binds to an antigen can be grafted into the framework regions of a second antibody having certain beneficial characteristics attributable to the framework regions, for example, humanized sequences. The resulting antibody retains the binding specificity of the first antibody while also acquiring the beneficial characteristics of the framework regions of the second antibody, such as reduced immunogenicity in humans.

Also, as discussed above, certain embodiments of the disclosure provide antibodies having humanized framework regions. Therefore, CDRs from any antibody that specifically binds to an antigen can be grafted into the framework regions disclosed herein to produce humanized antibodies having binding specificity for the antibody that provides the CDRs.

In certain embodiments, the present disclosure provides binding agents that specifically bind to CD30 protein. Such binding agents can be anti-CD30 antibodies. A CD-30 binding agent can be produced by grafting CDRs that confer to the variable domains of the binding agents the ability to specifically bind to CD30.

In one embodiment, the VH chain CDRs that confer to the binding agent the CD30 binding specificity comprise the sequences of SEQ ID NOs: 31-33; and the VL chain CDRs that confer to the binding agent the CD30 binding specificity comprise the sequences of SEQ ID NOs: 34-36. Any other combinations of CDRs that confer to the binding agent the CD30 binding specificity can also be grafted into the VH and VL domains described herein.

Thus, certain embodiments of the disclosure provide a binding agent that specifically binds to CD30 protein, the binding agent comprising:
i) a VH chain comprising a sequence selected from: SEQ ID NO: 6 and SEQ ID NO: 11; and
ii) a VL chain comprising a sequence selected from: SEQ ID NO: 21 and SEQ ID NO: 26.

For example, a binding agent that specifically binds to CD30, can comprise: 1) a VH chain comprising H-CDR1, H-CDR2, and H-CDR3 having the sequences of SEQ ID NOs: 31-33, respectively; and VL chain comprising L-CDR1, L-CDR2, and L-CDR3 having the sequences of SEQ ID NOs: 34-36, respectively.

In one embodiment, a binding agent that specifically binds to CD30, comprises: H-CDR1, H-CDR2, and H-CDR3 having the sequences of SEQ ID NOs: 31-33, and L-CDR1, L-CDR2, and L-CDR3 having the sequences of SEQ ID NOs: 34-36; and wherein, in the binding agent:
the VH chain comprises:
i) a HFR1 having the sequence of SEQ ID NO: 7, a HFR2 having the sequence of SEQ ID NO: 8, a HFR3 having the sequence of SEQ ID NO: 9, and a HFR4 having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and
the VL chain comprises:
i) a LFR1 having the sequence of SEQ ID NO: 22, a LFR2 having the sequence of SEQ ID NO: 23, a LFR3 having the sequence of SEQ ID NO: 24, and a LFR4 having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 25.

Certain embodiments of the disclosure provide a binding agent that specifically binds to CD30 protein, the binding agent comprising a combination of VH and VL chains selected from:
a) VH chain comprising SEQ ID NO: 6 and VL chain comprising SEQ ID NO: 21,
b) VH chain comprising SEQ ID NO: 6 and VL chain comprising SEQ ID NO: 26,
c) VH chain comprising SEQ ID NO: 11 and VL chain comprising SEQ ID NO: 21, and
d) VH chain comprising SEQ ID NO: 11 and VL chain comprising SEQ ID NO: 26.

According to some embodiments, an antibody of the present disclosure specifically binds to CD30 and competes for binding to CD30 with an antibody comprising:

Any suitable approach for determining whether a first antibody competes with a second antibody for binding to CD30 may be employed. Whether a first antibody “competes with” a second antibody for binding to a compound may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labelled. If the labeled antibody and the unlabeled antibody bind to separate and discrete sites on the compound, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the compound by about 50% or more, about 60% or more, about 70% or more, about 80% or more, about 85% or more, about 90% or more, about 95% or more, or about 99% or more. Details of procedures for carrying out such competition assays are well known in the art. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

According to some embodiments, an antibody of the present disclosure specifically binds to CD30 and comprises:

The HCDRs 1-3 and LCDRs 1-3 as defined by Chothia nomenclature. The HCDRs 1-3 and LCDRs 1-3 of the anti-CD30 antibodies disclosed herein as defined per the listed nomenclatures may be as described in the Table (Table 3) below.

TABLE 3
Domains and sequences in AC-10 antibody and H1,
H4, L2, and L4 variants identified by SEQ ID NOs.

| Antibody | FR1 | CDR1 | FR2 | CDR2 | FR3 | CDR3 | FR4 |
|---|---|---|---|---|---|---|---|
| H-AC-10 | 2 | 31 | 3 | 32 | 4 | 33 | 10 |
| H1 variant | 7 | 31 | 8 | 32 | 9 | 33 | 10 |
| H4 variant | 12 | 31 | 13 | 32 | 14 | 33 | 15 |
| L-AC-10 | 17 | 34 | 18 | AAS | 19 | 36 | 20 |
| L2 variant | 22 | 34 | 23 | AAS | 24 | 36 | 25 |
| L4 variant | 27 | 34 | 28 | AAS | 29 | 36 | 30 |

In certain embodiments, the VH chain of a CD30 binding agent comprises the HCDRs 1-3 as set forth in Table 3 and comprises in the framework regions amino acid sequences having 80% or greater, 85% or greater, 90% or greater, 95% or greater, 99% or greater, or 100% sequence identity to the amino acid sequences set forth in FR1 to FR4 as described in Table 3, with the exclusion of the residues mutated in the variants as compared to the parental AC-10 sequences, i.e., the mutated residues are retained, i.e., the mutated residues are retained. The mutations in the H1 and H4 variants as compared to the parental AC-10 sequences are shown in FIG. 1A and FIG. 1B, respectively.

In certain embodiments, the VL chain of a CD-30 binding agent comprises the LCDRs 1-3 as set forth herein in Table 3 and comprises in the framework regions amino acid sequences having 80% or greater, 85% or greater, 90% or greater, 95% or greater, 99% or greater, or 100% sequence identity to the amino acid sequences set forth in FR1 to FR4 as described in Table 3, with the exclusion of the residues mutated in the variants as compared to the parental AC-10 sequences, i.e., the mutated residues are retained. The mutations in the L2 and L4 variants as compared to the parental AC-10 sequences are shown in FIG. 1C and FIG. 1D, respectively.

The CD30-binding agents disclosed herein find use in a variety of research, diagnostic, and therapeutic applications, including for performing any of the methods described in U.S. Patent Application Nos. 20020064527, 20040136992, 20080003221, 20080206242, 20060177442, 20100239571, 20090214544, 20190218293, 20180280532, 20200095329, 20200095330, and 20200102399, the disclosure of which is incorporated herein by reference in its entirety for all purposes.

A “CD30 antigen” or “CD30 protein” can comprises an amino acid sequence having at least about 75%, at least about 80%, at least about 90%, at least about 95%, at least about 98%, at least about 99%, or 100%, amino acid sequence identity to the protein sequence described in NCBI Entry, GenBank Accession Number: CAC16652.1.

In some cases, the CD30 binding agents disclosed herein are modified, for example, by conjugation to an additional moiety. Various moieties that can be conjugated to binding agents, such as antibodies, for example, anti-CD30 antibodies disclosed herein are described below.

### Binding Agent Conjugates

The present disclosure provides a conjugate, such as a binding agent conjugate. In some instances, the binding agent is an antibody, and thus in these instances the present disclosure provides an antibody-drug conjugate (ADC). By “conjugate” is meant a polypeptide (e.g., a binding agent or an antibody) covalently attached to a moiety of interest (e.g., a drug or active agent). For example, a binding agent conjugate includes a binding agent (e.g., an antibody) covalently attached to a drug or active agent. In certain embodiments, the polypeptide (e.g., antibody) and the drug or active agent are bound to each other through one or more functional groups and covalent bonds. For example, the one or more functional groups and covalent bonds can include a linker as described herein.

In certain embodiments, the conjugate is a polypeptide conjugate, which includes a polypeptide conjugated to a moiety of interest. In certain embodiments, the moiety conjugated to the polypeptide can be any of a variety of moieties of interest such as, but not limited to, a detectable label, a drug, a water-soluble polymer, or a moiety for immobilization of the polypeptide to a membrane or a surface. In certain embodiments, the conjugate is a drug conjugate, where a polypeptide is conjugated to a drug or an active agent. Suitable drugs and active agents, and analogs and derivatives thereof, are described in more detail herein.

The moiety of interest (e.g., drug or active agent) can be conjugated to the polypeptide at any desired site of the polypeptide. Thus, the present disclosure provides, for example, a polypeptide having a moiety conjugated at a site at or near the C-terminus of the polypeptide. Other examples include a polypeptide having a moiety conjugated at a position at or near the N-terminus of the polypeptide. Examples also include a polypeptide having a moiety conjugated at a position between the C-terminus and the N-terminus of the polypeptide (e.g., at an internal site of the polypeptide). Combinations of the above are also possible where the polypeptide is conjugated to two or more moieties.

In certain embodiments, a conjugate of the present disclosure includes a drug or active agent conjugated to an amino acid residue of a polypeptide at the α-carbon of an amino acid residue. Stated another way, a conjugate includes a polypeptide where the side chain of one or more amino acid residues in the polypeptide is attached to a drug or active agent (e.g., attached to the drug or active agent through a linker as described herein). For example, a conjugate includes a polypeptide where the α-carbon of one or more amino acid residues in the polypeptide is attached to a drug or active agent (e.g., attached to the drug or active agent through a linker as described herein).

Embodiments of the present disclosure include conjugates where a polypeptide is conjugated to one or more moieties, such as 2 moieties, 3 moieties, 4 moieties, 5 moieties, 6 moieties, 7 moieties, 8 moieties, 9 moieties, or 10 or more moieties. The moieties may be conjugated to the polypeptide at one or more sites in the polypeptide. For example, one or more moieties may be conjugated to a single amino acid residue of the polypeptide. In some cases, one moiety is conjugated to an amino acid residue of the polypeptide. In other embodiments, two moieties may be conjugated to the same amino acid residue of the polypeptide. In other embodiments, a first moiety is conjugated to a first amino acid residue of the polypeptide and a second moiety is conjugated to a second amino acid residue of the polypeptide. Combinations of the above are also possible, for example where a polypeptide is conjugated to a first moiety at a first amino acid residue and conjugated to two other moieties at a second amino acid residue. Other combinations are also possible, such as, but not limited to, a polypeptide conjugated to first and second moieties at a first amino acid residue and conjugated to third and fourth moieties at a second amino acid residue, etc.

The one or more amino acid residues of the polypeptide that are conjugated to the one or more moieties may be naturally occurring amino acids, unnatural amino acids, or combinations thereof. For instance, the conjugate may include a moiety conjugated to a naturally occurring amino acid residue of the polypeptide. In other instances, the conjugate may include a moiety conjugated to an unnatural amino acid residue of the polypeptide. One or more moieties may be conjugated to the polypeptide at a single natural or unnatural amino acid residue as described above. One or more natural or unnatural amino acid residues in the polypeptide may be conjugated to the moiety or moieties as described herein. For example, two (or more) amino acid residues (e.g., natural or unnatural amino acid residues) in the polypeptide may each be conjugated to one or two moieties, such that multiple sites in the polypeptide are conjugated to the moieties of interest.

As described herein, a polypeptide may be conjugated to one or more moieties. In certain embodiments, the moiety of interest is a chemical entity, such as a drug or a detectable label. For example, a drug or active agent may be conjugated to the polypeptide, or in other embodiments, a detectable label may be conjugated to the polypeptide. Thus, for instance, embodiments of the present disclosure include, but are not limited to, the following: a conjugate of a polypeptide and a drug; a conjugate of a polypeptide and a detectable label; a conjugate of two or more drugs and a polypeptide; a conjugate of two or more detectable labels and a polypeptide; and the like.

In certain embodiments, the polypeptide and the moiety of interest are conjugated through a coupling moiety. For example, the polypeptide and the moiety of interest may each be bound (e.g., covalently bonded) to the coupling moiety, thus indirectly binding the polypeptide and the moiety of interest (e.g., a drug or active agent) together through the coupling moiety. In some cases, the coupling moiety includes a hydrazinyl-indolyl or a hydrazinyl-pyrrolo-pyridinyl compound, or a derivative of a hydrazinyl-indolyl or a hydrazinyl-pyrrolo-pyridinyl compound. For instance, a general scheme for coupling a moiety of interest (e.g., a drug or active agent) to a polypeptide through a hydrazinyl-indolyl or a hydrazinyl-pyrrolo-pyridinyl coupling moiety is shown in the general reaction scheme below. Hydrazinyl-indolyl and hydrazinyl-pyrrolo-pyridinyl coupling moiety are also referred to herein as a hydrazino-iso-Pictet-Spengler (HIPS) coupling moiety and an aza-hydrazino-iso-Pictet-Spengler (azaHIPS) coupling moiety, respectively.

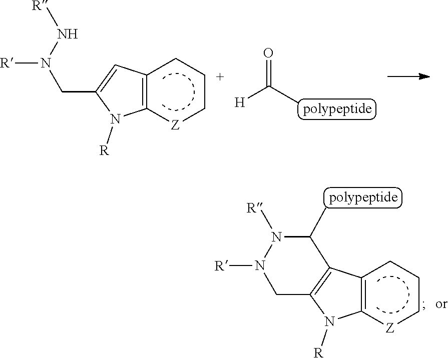

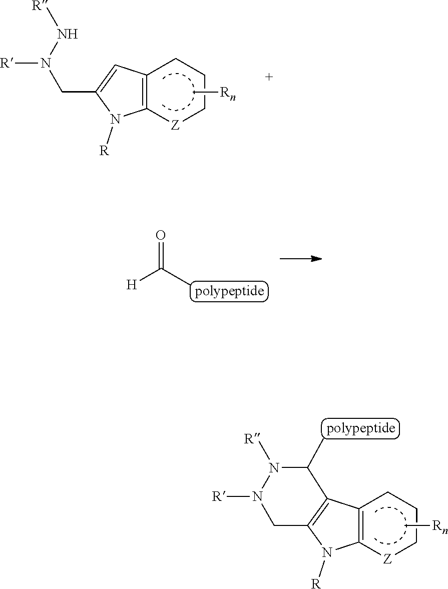

In the reaction scheme above, each R is the moiety of interest (e.g., drug or active agent) that is conjugated to the polypeptide, where n is an integer from 1 to 4. As shown in the reaction scheme above, a polypeptide that includes a 2-formylglycine residue (fGly) is reacted with a drug that has been modified to include a coupling moiety (e.g., a hydrazinyl-indolyl or a hydrazinyl-pyrrolo-pyridinyl coupling moiety) to produce a polypeptide conjugate attached to the coupling moiety, thus attaching the drug to the polypeptide through the coupling moiety.

As described herein, the moiety can be any of a variety of moieties such as, but not limited to, chemical entity, such as a detectable label, or a drug or active agent. R′ and R″ may each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. Z may be CR⁶¹, NR⁶², N, O or S, where R⁶¹ and R⁶² are each independently selected from any of the substituents described for R′ and R″ above.

Other hydrazinyl-indolyl or hydrazinyl-pyrrolo-pyridinyl coupling moieties are also possible, as shown in the conjugates and compounds described herein. For example, the hydrazinyl-indolyl or hydrazinyl-pyrrolo-pyridinyl coupling moieties may be attached (e.g., covalently attached) to a linker. As such, embodiments of the present disclosure include a hydrazinyl-indolyl or hydrazinyl-pyrrolo-pyridinyl coupling moiety attached to a drug or active agent through a linker. Various embodiments of the linker that may couple the hydrazinyl-indolyl or hydrazinyl-pyrrolo-pyridinyl coupling moiety to the drug or active agent are described in detail herein.

Additional hydrazinyl-indolyl or hydrazinyl-pyrrolo-pyridinyl conjugation moieties are also possible, as shown in the conjugates and compounds described herein. For example, the hydrazinyl-indolyl or hydrazinyl-pyrrolo-pyridinyl conjugation moieties may be attached (e.g., covalently attached) to two or more linkers. As such, embodiments of the present disclosure include a hydrazinyl-indolyl or hydrazinyl-pyrrolo-pyridinyl conjugation moiety attached to two or more drugs or active agents each through a corresponding linker. Thus, conjugates of the present disclosure may include two or more linkers, where each linker attaches a corresponding drug or active agent to the hydrazinyl-indolyl or hydrazinyl-pyrrolo-pyridinyl conjugation moiety. Accordingly, the hydrazinyl-indolyl or hydrazinyl-pyrrolo-pyridinyl conjugation moiety and two or more linkers may be viewed overall as a “branched linker”, where the hydrazinyl-indolyl or hydrazinyl-pyrrolo-pyridinyl conjugation moiety is attached to two of more “branches”, where each branch includes a linker attached to a drug or active agent.

Combinations of the same of different payloads may be conjugated to the polypeptide through the branched linker. In certain embodiments, the two payloads (e.g., drugs, active agents or detectable labels) attached to the branched linker are the same payload (e.g., drug, active agent or detectable label). For example, a first branch of a branched linker may be attached to a payload (e.g., drug, active agent or detectable label) and a second branch of the branched linker may be attached to the same payload (e.g., drug, active agent or detectable label) as the first branch.

In other embodiments, the two payloads (e.g., drugs, active agents or detectable labels) attached to the branched linker are different payloads (e.g., drugs, active agents or detectable labels). For example, a first branch of a branched linker may be attached to a first payload (e.g., a first drug, active agent or detectable label) and a second branch of the branched linker may be attached to a second payload (e.g., a second drug, active agent or detectable label) different from the first payload (e.g., the first drug, active agent or detectable label) attached to the first branch.

In certain embodiments, the polypeptide (e.g., antibody) may be conjugated to a moiety of interest, where one or more amino acids of the polypeptide are modified before conjugation to the moiety of interest. Modification of one or more amino acids of the polypeptide may produce a polypeptide that contains one or more reactive groups suitable for conjugation to the moiety of interest. In some cases, the polypeptide may include one or more modified amino acid residues to provide one or more reactive groups suitable for conjugation to the moiety of interest (e.g., a moiety that includes a coupling moiety, such as a hydrazinyl-indolyl or a hydrazinyl-pyrrolo-pyridinyl coupling moiety as described above). For example, an amino acid of the polypeptide may be modified to include a reactive aldehyde group (e.g., a reactive aldehyde). A reactive aldehyde may be included in an “aldehyde tag” or “ald-tag”, which as used herein refers to an amino acid sequence produced from a sulfatase motif (e.g., L(C/S)TPSR) that has been converted by action of a formylglycine generating enzyme (FGE) to contain a 2-formylglycine residue (referred to herein as “fGly”). The fGly residue generated by an FGE may also be referred to as a “formylglycine”. Stated differently, the term “aldehyde tag” is used herein to refer to an amino acid sequence that includes a “converted” sulfatase motif (i.e., a sulfatase motif in which a cysteine or serine residue has been converted to fGly by action of an FGE, e.g., L(fGly)TPSR). A converted sulfatase motif may be produced from an amino acid sequence that includes an “unconverted” sulfatase motif (i.e., a sulfatase motif in which the cysteine or serine residue has not been converted to fGly by an FGE, but is capable of being converted, e.g., an unconverted sulfatase motif with the sequence: L(C/S)TPSR). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 8,729,232, the disclosures of each of which are incorporated herein by reference.

In some cases, to produce the conjugate, the polypeptide containing the fGly residue may be conjugated to the moiety of interest by reaction of the fGly with a compound (e.g., a compound containing a hydrazinyl-indolyl or a hydrazinyl-pyrrolo-pyridinyl coupling moiety, as described above). For example, an fGly-containing polypeptide may be contacted with a reactive partner-containing drug under conditions suitable to provide for conjugation of the drug to the polypeptide. In some instances, the reactive partner-containing drug may include a hydrazinyl-indolyl or a hydrazinyl-pyrrolo-pyridinyl coupling moiety as described above. For example, a drug or active agent may be modified to include a hydrazinyl-indolyl or a hydrazinyl-pyrrolo-pyridinyl coupling moiety. In some cases, the drug or active agent is attached to a hydrazinyl-indolyl or a hydrazinyl-pyrrolo-pyridinyl coupling moiety, such as covalently attached to a hydrazinyl-indolyl or a hydrazinyl-pyrrolo-pyridinyl coupling moiety through a linker, as described in detail herein.

In certain embodiments, a conjugate of the present disclosure includes a polypeptide (e.g., a binding agent or an antibody) having at least one amino acid residue that has been attached to a moiety of interest (e.g., drug or active agent). In order to make the conjugate, an amino acid residue of the polypeptide may be modified and then coupled to a drug or active agent containing a hydrazinyl-indolyl or a hydrazinyl-pyrrolo-pyridinyl coupling moiety as described above. In certain embodiments, an amino acid residue of the polypeptide is a cysteine or serine residue that is converted to an fGly residue, as described above. In certain embodiments, the amino acid residue (e.g., fGly residue) is conjugated to a drug or active agent containing a hydrazinyl-indolyl or a hydrazinyl-pyrrolo-pyridinyl coupling moiety as described above to provide a conjugate of the present disclosure where the drug or active agent is conjugated to the polypeptide through the hydrazinyl-indolyl or hydrazinyl-pyrrolo-pyridinyl coupling moiety. As used herein, the term fGly′ refers to the amino acid residue of the polypeptide (e.g., binding agent or antibody) that is coupled to the moiety of interest (e.g., drug or active agent).

In certain embodiments, the conjugate includes a polypeptide (e.g., binding agent or antibody) having at least one amino acid residue attached to a linker as described herein, which in turn is attached to a drug or active agent. For instance, the conjugate may include a polypeptide (e.g., binding agent or antibody) having at least one amino acid residue (fGly′) that is conjugated to a drug or active agent.

### Conjugates of Formula (I)

Aspects of the present disclosure include a conjugate of formula (I):

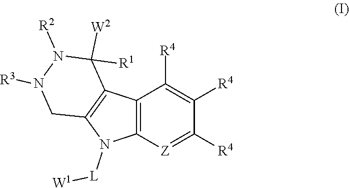

wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker;
W¹ is a drug (or active agent); and
W² is a binding agent as described herein.

In certain embodiments, Z is CR⁴ or N. In certain embodiments, Z is CR⁴. In certain embodiments, Z is N.

In certain embodiments, R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl.

In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is methyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl.

In certain embodiments, R² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is alkoxy or substituted alkoxy. In certain embodiments, R² is amino or substituted amino. In certain embodiments, R² is carboxyl or carboxyl ester. In certain embodiments, R² is acyl or acyloxy. In certain embodiments, R² is acyl amino or amino acyl. In certain embodiments, R² is alkylamide or substituted alkylamide. In certain embodiments, R² is sulfonyl. In certain embodiments, R² is thioalkoxy or substituted thioalkoxy. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is alkoxy or substituted alkoxy. In certain embodiments, R³ is amino or substituted amino. In certain embodiments, R³ is carboxyl or carboxyl ester. In certain embodiments, R³ is acyl or acyloxy. In certain embodiments, R³ is acyl amino or amino acyl. In certain embodiments, R³ is alkylamide or substituted alkylamide. In certain embodiments, R³ is sulfonyl. In certain embodiments, R³ is thioalkoxy or substituted thioalkoxy. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are optionally cyclically linked to form a 5 or 6-membered heterocyclyl. In certain embodiments, R² and R³ are cyclically linked to form a 5 or 6-membered heterocyclyl. In certain embodiments, R² and R³ are cyclically linked to form a 5-membered heterocyclyl. In certain embodiments, R² and R³ are cyclically linked to form a 6-membered heterocyclyl.

In certain embodiments, each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R⁴ are described in more detail as follows. In certain embodiments, R⁴ is hydrogen. In certain embodiments, each R⁴ is hydrogen. In certain embodiments, R⁴ is halogen, such as F, Cl, Br or I. In certain embodiments, R⁴ is F. In certain embodiments, R⁴ is Cl. In certain embodiments, R⁴ is Br. In certain embodiments, R⁴ is I. In certain embodiments, R⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁴ is methyl. In certain embodiments, R⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁴ is alkynyl or substituted alkynyl. In certain embodiments, R⁴ is alkoxy or substituted alkoxy. In certain embodiments, R⁴ is amino or substituted amino. In certain embodiments, R⁴ is carboxyl or carboxyl ester. In certain embodiments, R⁴ is acyl or acyloxy. In certain embodiments, R⁴ is acyl amino or amino acyl. In certain embodiments, R⁴ is alkylamide or substituted alkylamide. In certain embodiments, R⁴ is sulfonyl. In certain embodiments, R⁴ is thioalkoxy or substituted thioalkoxy. In certain embodiments, R⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, W¹ is a drug. Further description of the drug is found in the disclosure herein.

In certain embodiments, W² is a binding agent as described herein. In certain embodiments, W² comprises one or more fGly′ residues as described herein. In certain embodiments, the binding agent is attached to the rest of the conjugate through an fGly′ residue as described herein. Further description of the binding agents that find use in the subject conjugates is found in the disclosure herein.

In certain embodiments, the compounds of formula (I) include a linker, L. The linker may be utilized to bind the conjugation moiety (e.g., a hydrazinyl-indolyl or a hydrazinyl-pyrrolo-pyridinyl conjugation moiety) to one or more moieties of interest. The linker may be bound (e.g., covalently bonded) to the conjugation moiety (e.g., as described herein) at any convenient position. For example, the linker may attach a hydrazinyl-indolyl or a hydrazinyl-pyrrolo-pyridinyl conjugation moiety to a drug. The hydrazinyl-indolyl or hydrazinyl-pyrrolo-pyridinyl coupling moiety may be used to conjugate the linker (and thus the drug) to a polypeptide, such as an antibody or binding agent as described herein. For example, the conjugation moiety may be used to conjugate the linker (and thus the drug) to a modified amino acid residue of the polypeptide, such as an fGly residue of an antibody or binding agent as described herein.

In certain embodiments, L attaches the conjugation moiety to W¹, and thus the conjugation moiety is indirectly bonded to W¹ through the linker L. As described above, W¹ is a drug, and thus L attaches the conjugation moiety to a drug, e.g., the conjugation moiety is indirectly bonded to the drug through the linker, L.

Any convenient linker may be utilized in the subject conjugates. In certain embodiments, L includes a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, L includes an alkyl or substituted alkyl group. In certain embodiments, L includes an alkenyl or substituted alkenyl group. In certain embodiments, L includes an alkynyl or substituted alkynyl group. In certain embodiments, L includes an alkoxy or substituted alkoxy group. In certain embodiments, L includes an amino or substituted amino group. In certain embodiments, L includes a carboxyl or carboxyl ester group. In certain embodiments, L includes an acyl amino group. In certain embodiments, L includes an alkylamide or substituted alkylamide group. In certain embodiments, L includes an aryl or substituted aryl group. In certain embodiments, L includes a heteroaryl or substituted heteroaryl group. In certain embodiments, L includes a cycloalkyl or substituted cycloalkyl group. In certain embodiments, L includes a heterocyclyl or substituted heterocyclyl group.

In certain embodiments, L includes a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L is a linker described by the formula:

-(L¹)ₐ-(L²)_{b}-(L³)_{c}-(L⁴)_{d}-(L⁵)ₑ-(L⁶)_{f}-,

wherein L¹, L², L³, L⁴, L⁵ and L⁶ are each independently a linker subunit, and a, b, c, d, e and f are each independently 0 or 1, wherein the sum of a, b, c, d, e and f is 1 to 6.

In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the hydrazinyl-indolyl or the hydrazinyl-pyrrolo-pyridinyl conjugation moiety (e.g., as shown in formula (I) above). In certain embodiments, the linker subunit L², if present, is attached to drug. In certain embodiments, the linker subunit L³, if present, is attached to the drug. In certain embodiments, the linker subunit L⁴, if present, is attached to the drug. In certain embodiments, the linker subunit L⁵, if present, is attached to the drug. In certain embodiments, the linker subunit L⁶, if present, is attached to the drug.

Any convenient linker subunits may be utilized in the linker L. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵ (if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L is a linker comprising -(L¹)ₐ-(L²)_{b}-(L³)_{c}-(L⁴)_{d}-(L⁵)ₑ-(L⁶)_{f}-, where:
-(L¹)ₐ is -(T¹-V¹)ₐ—;
-(L²)_{b}- is -(T²-V²)_{b}—;
-(L³)_{c}- is -(T³-V³)_{c}—;
-(L⁴)_{d}- is -(T⁴-V⁴)_{d}—;
-(L⁵)ₑ- is -(T⁵-V⁵)ₑ—; and
-(L⁶)_{f}- is -(T⁶-V⁶)_{f}—,
wherein T¹, T², T³, T⁴, T⁵ and T⁶, if present, are tether groups;
V¹, V², V³, V⁴, V⁵ and V⁶, if present, are covalent bonds or linking functional groups; and
a, b, c, d, e and f are each independently 0 or 1, wherein the sum of a, b, c, d, e and f is 1 to 6.

As described above, in certain embodiments, L¹ is attached to the hydrazinyl-indolyl or the hydrazinyl-pyrrolo-pyridinyl conjugation moiety (e.g., as shown in formula (I) above). As such, in certain embodiments, T¹ is attached to the hydrazinyl-indolyl or the hydrazinyl-pyrrolo-pyridinyl conjugation moiety (e.g., as shown in formula (I) above). In certain embodiments, V¹ is attached to the drug. In certain embodiments, L², if present, is attached to the drug. As such, in certain embodiments, T², if present, is attached to the drug, or V², if present, is attached to the drug. In certain embodiments, L³, if present, is attached to the drug. As such, in certain embodiments, T³, if present, is attached to the drug, or V³, if present, is attached to the drug. In certain embodiments, L⁴, if present, is attached to the drug. As such, in certain embodiments, T⁴, if present, is attached to the drug, or V⁴, if present, is attached to the drug. In certain embodiments, L⁵, if present, is attached to the drug. As such, in certain embodiments, T⁵, if present, is attached to the drug, or V⁵, if present, is attached to the drug. In certain embodiments, L⁶, if present, is attached to the drug. As such, in certain embodiments, T⁶, if present, is attached to the drug, or V⁶, if present, is attached to the drug.

Regarding the tether groups, T¹, T², T³, T⁴, T⁵ and T⁶, any convenient tether groups may be utilized in the subject linkers. In some embodiments, T¹, T², T³, T⁴, T⁵ and T⁶ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, where each w is an integer from 1 to 20, each n is an integer from 1 to 30, each p is an integer from 1 to 20, and each m is an integer from 1 to 12.

In certain embodiments, the tether group (e.g., T¹, T², T³, T⁴, T⁵ and/or T⁶) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₂-alkylene (e.g., CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₂-alkylene.

In certain embodiments, the tether group (e.g., T¹, T², T³, T⁴, T⁵ and/or T⁶) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵ and T⁶) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵ and/or T⁶) includes a heteroaryl or substituted heteroaryl. In some instances, the tether group (e.g., T¹, T², T³, T⁴, T⁵ and T⁶) includes a cycloalkyl or substituted cycloalkyl. In some instances, the tether group (e.g., T¹, T², T³, T⁴, T⁵ and T⁶) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵ and/or T⁶) includes an ethylene diamine (EDA) moiety, e.g., an EDA containing tether group. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

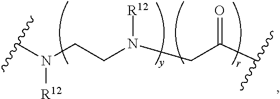

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T⁵ and/or T⁶) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

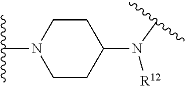

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

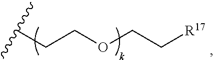

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH.

In certain embodiments, a tether group (e.g., T¹, T², T³, T⁴, T⁵ and/or T⁶) includes (PEG)ₙ, where (PEG)ₙ is a polyethylene glycol or a modified polyethylene glycol linking unit. In certain embodiments, (PEG)ₙ is described by the structure:

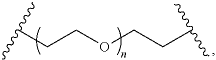

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵ and/or T⁶) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵ and/or T⁶) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Len or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T¹, T², T³, T⁴, T⁵ and/or T⁶) includes a moiety described by the formula —(CR¹³OH)ₘ—, where m is 0 or n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, m is 1. In certain embodiments, m is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³.

In certain embodiments, a tether group (e.g., T¹, T², T³, T⁴, T⁵ and/or T⁶) includes a meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), or para-hydroxy-phenyl (PHP).

In some embodiments, a tether includes a MABO group described by the following structure:

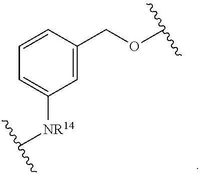

In some embodiments, a tether includes a MABC group described by the following structure:

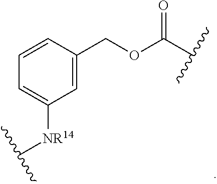

In some embodiments, a tether includes a PABO group described by the following structure:

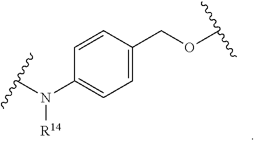

In some embodiments, a tether includes a PABC group described by the following structure

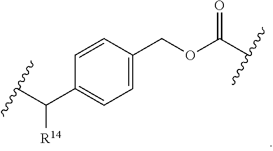

In some embodiments, a tether includes a PAB group described by the following structure:

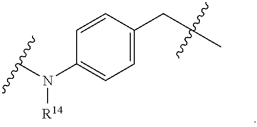

In some embodiments, a tether includes a PABA group described by the following structure:

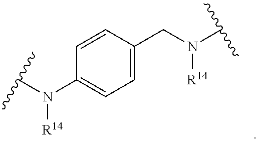

In some embodiments, a tether includes a PAP group described by the following structure:

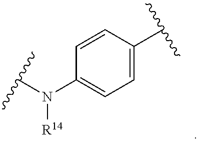

In some embodiments, a tether includes a PHP group described by the following structure:

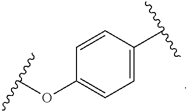 [SMILES: *Oc1ccc(*)cc1]

In certain embodiments, each R¹⁴ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁴ is hydrogen. In certain embodiments, each R¹⁴ is hydrogen. In certain embodiments, R¹⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁴ is alkynyl or substituted alkynyl. In certain embodiments, R¹⁴ is alkoxy or substituted alkoxy. In certain embodiments, R¹⁴ is amino or substituted amino. In certain embodiments, R¹⁴ is carboxyl or carboxyl ester. In certain embodiments, R¹⁴ is acyl or acyloxy. In certain embodiments, R¹⁴ is acyl amino or amino acyl. In certain embodiments, R¹⁴ is alkylamide or substituted alkylamide. In certain embodiments, R¹⁴ is sulfonyl. In certain embodiments, R¹⁴ is thioalkoxy or substituted thioalkoxy. In certain embodiments, R¹⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In some embodiments of the MABO, MABC, PABO, PABC, PAB, PABA, PAP, and PHP tether structures shown above, the phenyl ring may be substituted with one or more additional groups selected from halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments of the linker L, one or more of the tether groups T¹, T², T³, T⁴, T⁵ or T⁶ is each optionally substituted with a glycoside or glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments, the MABO, MABC, PABO, PABC, PAB, PABA, PAP, and PHP tether structures shown above may be substituted with one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

For example, in some embodiments, the glycoside or glycoside derivative can be selected from the following structures:

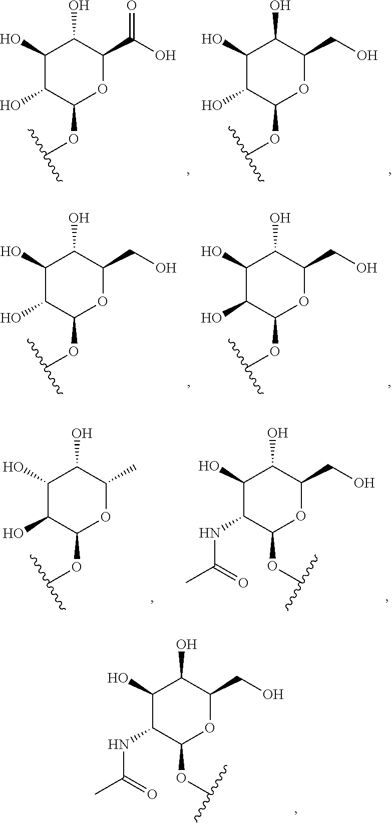

Regarding the linking functional groups, V¹, V², V³, V⁴, V⁵ and V⁶, any convenient linking functional groups may be utilized in the linker L. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵.

In certain embodiments, the tether group includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

As described above, in some embodiments, L is a linker comprising -(T¹-V¹)ₐ-(T²-V²)_{b}-(T³-V³)_{c}-(T⁴-V⁴)_{d}-(T⁵-V⁵)ₑ-(T⁶-V⁶)_{f}—, where a, b, c, d, e and f are each independently 0 or 1, where the sum of a, b, c, d, e and f is 1 to 6.

In some embodiments, in the linker L:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), MABO, MABC, PABO, PABC, PAB, PABA, PAP, PHP, an acetal group, a disulfide, a hydrazine, and an ester; and
V¹, V², V³, V⁴, V⁵ and V⁶ are each independently selected from a covalent bond, —CO—, —NR¹⁵, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
wherein:
   (PEG)ₙ is

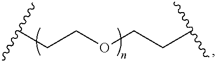

where n is an integer from 1 to 30;
EDA is an ethylene diamine moiety having the following structure:

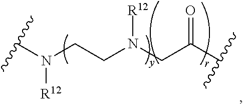

where y is an integer from 1 to 6 and r is 0 or 1;
4-amino-piperidine (4AP) is

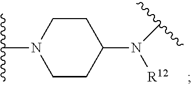

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In certain embodiments, T, T², T³, T⁴, T⁵ and T⁶ and V¹, V², V³, V⁴, V⁵ and V⁶ are selected from the following:

wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0; or
   wherein:
T¹ is (C₁-C₁₂)alkyl and V¹ is —CO—;
T² is 4AP and V² is —CO—;
T³ is (C₁-C₁₂)alkyl and V³ is —CO—;
d, e and f are each 0.

For example, in certain embodiments, the conjugate of formula (I) has a structure selected from the following:

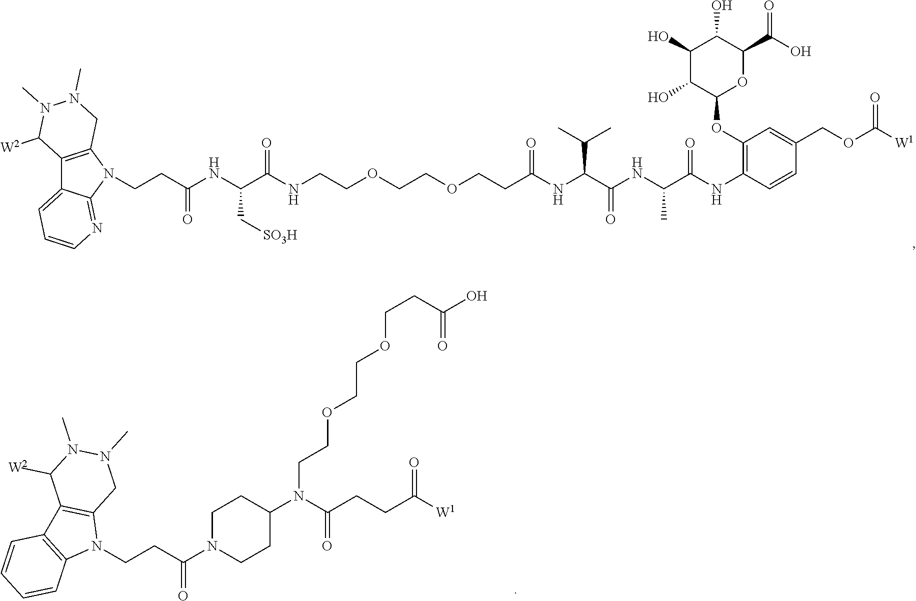

In certain embodiments, the left-hand side of the linker structure is attached to the hydrazinyl-indolyl or the hydrazinyl-pyrrolo-pyridinyl conjugation moiety, and the right-hand side of the linker structure is attached to the drug W¹, e.g., as shown above.

In certain embodiments, the conjugate is an antibody-drug conjugate where the antibody and the drug are linked together by a linker (e.g., L), as described above. In some instances, the linker is a cleavable linker. A cleavable linker is a linker that includes one or more cleavable moieties, where the cleavable moiety includes one or more bonds that can dissociate under certain conditions, thus separating the cleavable linker into two or more separatable portions. For example, the cleavable moiety may include one or more covalent bonds, which under certain conditions, can dissociate or break apart to separate the cleavable linker into two or more portions. As such a cleavable linker can be included in an antibody-drug conjugate, such that under appropriate conditions, the cleavable linker is cleaved to separate or release the drug from the antibody at a desired target site of action for the drug.

In some instances, the cleavable linker includes two cleavable moieties, such as a first cleavable moiety and a second cleavable moiety. The cleavable moieties can be configured such that cleavage of both cleavable moieties is needed in order to separate or release the drug from the antibody at a desired target site of action for the drug. For example, cleavage of the cleavable linker can be achieved by initially cleaving one of the two cleavable moieties and then cleaving the other of the two cleavable moieties. In certain embodiments, the cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (e.g., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as the protease enzyme cathepsin B, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (e.g., the site of the cancer (and overexpressed enzyme)). Thus, in some embodiments, the enzymatically cleavable moiety is a cleavable moiety (e.g., a peptide) that can be cleaved by an enzyme that is overexpressed in cancer cells. For instance, the enzyme can be the protease enzyme cathepsin B. As such, in some instances, the enzymatically cleavable moiety is a cleavable moiety (e.g., a peptide) that can be cleaved by a protease enzyme, such as cathepsin B.

In certain embodiments, the enzymatically cleavable moiety is a peptide. The peptide can be any peptide suitable for use in the cleavable linker and that can be cleaved through the enzymatic action of an enzyme. Non-limiting examples of peptides that can be used as an enzymatically cleavable moiety include, for example, Val-Ala, Phe-Lys, and the like. For example, the first cleavable moiety described above (e.g., the cleavable moiety protected from premature cleavage by the second cleavable moiety) can include a peptide. The presence of uncleaved second cleavable moiety can protect the first cleavable moiety (peptide) from cleavage by a protease enzyme (e.g., cathepsin B), and thus substantially reduce or prevent premature release of the drug from the antibody until the antibody-drug conjugate is at or near the desired target site of action for the drug. In some instances, one of the amino acid residues of the peptide that comprises the first cleavable moiety is linked to or includes a substituent, where the substituent comprises the second cleavable moiety. In some instances, the second cleavable moiety includes a glycoside.

In some embodiments, the enzymatically cleavable moiety is sugar moiety, such as a glycoside (or glyosyl). In some cases, the glycoside can facilitate an increase in the hydrophilicity of the cleavable linker as compared to a cleavable linker that does not include the glycoside. The glycoside can be any glycoside or glycoside derivative suitable for use in the cleavable linker and that can be cleaved through the enzymatic action of an enzyme. For example, the second cleavable moiety (e.g., the cleavable moiety that protects the first cleavable moiety from premature cleavage) can be a glycoside. For instance, in some embodiments, the first cleavable moiety includes a peptide and the second cleavable moiety includes a glycoside. In certain embodiments, the second cleavable moiety is a glycoside or glycoside derivative selected from a glucuronide, a galactoside, a glucoside, a mannoside, a fucoside, O-GlcNAc, and O-GalNAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is 0-GlcNAc. In some instances, the second cleavable moiety is O-GalNAc.

The glycoside can be attached (e.g., covalently bonded) to the cleavable linker through a glycosidic bond. The glycosidic bond can link the glycoside to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some instances, the glycosidic bond is an O-glycosidic bond (an O-glycoside). In some cases, the glycoside can be cleaved from the cleavable linker it is attached to by an enzyme (e.g., through enzymatically-mediated hydrolysis of the glycosidic bond). A glycoside can be removed or cleaved from the cleavable linker by any convenient enzyme that is able to carry out the cleavage (hydrolysis) of the glycosidic bond that attaches the glycoside to the cleavable linker. An example of an enzyme that can be used to mediate the cleavage (hydrolysis) of the glycosidic bond that attaches the glycoside to the cleavable linker is a glucuronidase, a glycosidase, such as a galactosidase, a glucosidase, a mannosidase, a fucosidase, and the like. Other suitable enzymes may also be used to mediate the cleavage (hydrolysis) of the glycosidic bond that attaches the glycoside to the cleavable linker. In some cases, the enzyme used to mediate the cleavage (hydrolysis) of the glycosidic bond that attaches the glycoside to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

In certain embodiments, the conjugate of formula (I) has a structure selected from the following:

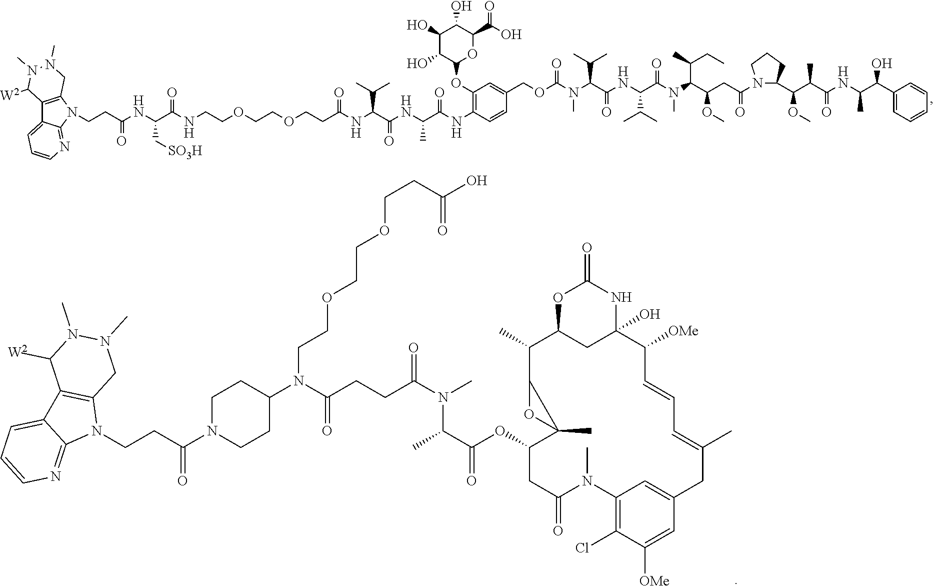

Any of the chemical entities, drugs, linkers and coupling moieties set forth in the description and structures above may be adapted for use in the subject conjugates.

Additional disclosure related to hydrazinyl-indolyl and hydrazinyl-pyrrolo-pyridinyl compounds and methods for producing a conjugate is found in U.S. Pat. Nos. 9,310,374 and 9,493,413, the disclosures of each of which are incorporated herein by reference. Additional disclosure related to cleavable linkers is found in U.S. Provisional Application No. 63/214,525, filed Jun. 24, 2021, the disclosure of which is incorporated herein by reference.

In certain embodiments of formula (I), the conjugate is a conjugate of the formula (Ia):

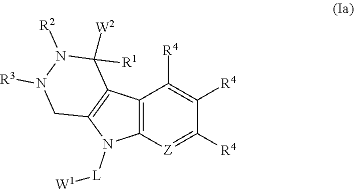

wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker comprising -(T¹-V¹)ₐ-(T²-V²)_{b}-(T³-V³)_{c}-(T⁴-V⁴)_{d}—, wherein a, b, c and d are each independently 0 or 1, where the sum of a, b, c and d is 1 to 4;
T¹, T², T³ and T⁴ are each independently selected from (C₁-C₁₂)alkyl, substituted (C₁-C₁₂)alkyl, (EDA)_{w}, (PEG)ₙ, (AA)ₚ, —(CR¹³OH)ₕ—, piperidin-4-amino (4AP), an acetal group, a hydrazine, a disulfide, and an ester, wherein EDA is an ethylene diamine moiety, PEG is a polyethylene glycol or a modified polyethylene glycol, and AA is an amino acid residue, wherein w is an integer from 1 to 20, n is an integer from 1 to 30, p is an integer from 1 to 20, and h is an integer from 1 to 12;
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl; W¹ is a drug (or active agent); and W² is a binding agent as described herein.

In certain embodiments, Z is CR⁴ or N. In certain embodiments, Z is CR⁴. In certain embodiments, Z is N.

In certain embodiments, R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is methyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl.

In certain embodiments, R² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is alkoxy or substituted alkoxy. In certain embodiments, R² is amino or substituted amino. In certain embodiments, R² is carboxyl or carboxyl ester. In certain embodiments, R² is acyl or acyloxy. In certain embodiments, R² is acyl amino or amino acyl. In certain embodiments, R² is alkylamide or substituted alkylamide. In certain embodiments, R² is sulfonyl. In certain embodiments, R² is thioalkoxy or substituted thioalkoxy. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is alkoxy or substituted alkoxy. In certain embodiments, R³ is amino or substituted amino. In certain embodiments, R³ is carboxyl or carboxyl ester. In certain embodiments, R³ is acyl or acyloxy. In certain embodiments, R³ is acyl amino or amino acyl. In certain embodiments, R³ is alkylamide or substituted alkylamide. In certain embodiments, R³ is sulfonyl. In certain embodiments, R³ is thioalkoxy or substituted thioalkoxy. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are optionally cyclically linked to form a 5 or 6-membered heterocyclyl. In certain embodiments, R² and R³ are cyclically linked to form a 5 or 6-membered heterocyclyl. In certain embodiments, R² and R³ are cyclically linked to form a 5-membered heterocyclyl. In certain embodiments, R² and R³ are cyclically linked to form a 6-membered heterocyclyl.

In certain embodiments, each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R⁴ are described in more detail as follows. In certain embodiments, R⁴ is hydrogen. In certain embodiments, each R⁴ is hydrogen. In certain embodiments, R⁴ is halogen, such as F, Cl, Br or I. In certain embodiments, R⁴ is F. In certain embodiments, R⁴ is Cl. In certain embodiments, R⁴ is Br. In certain embodiments, R⁴ is I. In certain embodiments, R⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁴ is methyl. In certain embodiments, R⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁴ is alkynyl or substituted alkynyl. In certain embodiments, R⁴ is alkoxy or substituted alkoxy. In certain embodiments, R⁴ is amino or substituted amino. In certain embodiments, R⁴ is carboxyl or carboxyl ester. In certain embodiments, R⁴ is acyl or acyloxy. In certain embodiments, R⁴ is acyl amino or amino acyl. In certain embodiments, R⁴ is alkylamide or substituted alkylamide. In certain embodiments, R⁴ is sulfonyl. In certain embodiments, R⁴ is thioalkoxy or substituted thioalkoxy. In certain embodiments, R⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, W¹ is a drug or active agent. Further description of drugs and active agents suitable for the subject conjugates is found in the disclosure herein.

In certain embodiments, W² is a binding agent as described herein. For example, in some cases, W² is an antibody. In certain embodiments, W² comprises one or more fGly′ residues as described herein. In certain embodiments, the binding agent is attached to the rest of the conjugate through an fGly′ residue as described herein. Further description of binding agents and antibodies that find use in the subject conjugates is found in the disclosure herein.

In certain embodiments, the compounds of formula (Ia) include a linker, L. The linker may be utilized to bind a coupling moiety to one or more moieties of interest and/or one or more polypeptides. In some embodiments, the linker binds a coupling moiety to either a polypeptide or a chemical entity. The linker may be bound (e.g., covalently bonded) to the coupling moiety (e.g., as described herein) at any convenient position. For example, the linker may attach a hydrazinyl-indolyl or a hydrazinyl-pyrrolo-pyridinyl coupling moiety to a drug or active agent. The hydrazinyl-indolyl or hydrazinyl-pyrrolo-pyridinyl coupling moiety may be used to conjugate the linker (and thus the drug or active agent) to a polypeptide, such as a binding agent or an antibody. For example, the coupling moiety may be used to conjugate the linker (and thus the drug or active agent) to an amino acid residue of the polypeptide, such as an fGly reside of a binding agent or an antibody.

In certain embodiments, L attaches the coupling moiety to W¹, and thus the coupling moiety is indirectly bonded to W¹ through the linker L. As described above, W¹ is a drug or active agent, and thus L attaches the coupling moiety to a drug or active agent, e.g., the coupling moiety is indirectly bonded to the drug or active agent through the linker, L.

Any convenient linkers may be utilized in the subject conjugates and compounds. In certain embodiments, L includes a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, L includes an alkyl or substituted alkyl group. In certain embodiments, L includes an alkenyl or substituted alkenyl group. In certain embodiments, L includes an alkynyl or substituted alkynyl group. In certain embodiments, L includes an alkoxy or substituted alkoxy group. In certain embodiments, L includes an amino or substituted amino group. In certain embodiments, L includes a carboxyl or carboxyl ester group. In certain embodiments, L includes an acyl amino group. In certain embodiments, L includes an alkylamide or substituted alkylamide group. In certain embodiments, L includes an aryl or substituted aryl group. In certain embodiments, L includes a heteroaryl or substituted heteroaryl group. In certain embodiments, L includes a cycloalkyl or substituted cycloalkyl group. In certain embodiments, L includes a heterocyclyl or substituted heterocyclyl group.

In certain embodiments, L includes a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L is a linker described by the formula (L¹)ₐ(L²)_{b}-(L³)_{c}-(L⁴)_{d}-, wherein L¹, L², L³ and L⁴ are each independently a linker unit, and a, b, c and d are each independently 0 or 1, wherein the sum of a, b, c and d is 1 to 4.

In certain embodiments, the sum of a, b, c and d is 1. In certain embodiments, the sum of a, b, c and d is 2. In certain embodiments, the sum of a, b, c and d is 3. In certain embodiments, the sum of a, b, c and d is 4. In certain embodiments, a, b, c and d are each 1. In certain embodiments, a, b and c are each 1 and d is 0. In certain embodiments, a and b are each 1 and c and d are each 0. In certain embodiments, a is 1 and b, c and d are each 0.

In certain embodiments, L¹ is attached to the hydrazinyl-indolyl or the hydrazinyl-pyrrolo-pyridinyl coupling moiety (e.g., as shown in formula (Ia) above). In certain embodiments, L², if present, is attached to W¹. In certain embodiments, L³, if present, is attached to W¹. In certain embodiments, L⁴, if present, is attached to W¹.

Any convenient linker units may be utilized in the subject linkers. Linker units of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³ and L⁴ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L is a linker comprising -(L¹)ₐ-(L²)_{b}-(L³)_{c}-(L⁴)_{d}-, where:
-(L¹)ₐ is -(T¹-V¹)ₐ—;
-(L²)_{b}- is -(T²-V²)_{b}—;
-(L³)_{c}- is -(T³-V³)_{c}—; and
-(L⁴)_{d}- is -(T⁴-V⁴)_{d}—,
wherein T¹, T², T³ and T⁴, if present, are tether groups;
V¹, V², V³ and V⁴, if present, are covalent bonds or linking functional groups; and
a, b, c and d are each independently 0 or 1, wherein the sum of a, b, c and d is 1 to 4.

As described above, in certain embodiments, L¹ is attached to the hydrazinyl-indolyl or the hydrazinyl-pyrrolo-pyridinyl coupling moiety (e.g., as shown in formula (Ia) above). As such, in certain embodiments, T¹ is attached to the hydrazinyl-indolyl or the hydrazinyl-pyrrolo-pyridinyl coupling moiety (e.g., as shown in formula (Ia) above). In certain embodiments, V¹ is attached to W¹ (the drug or active agent). In certain embodiments, L², if present, is attached to W¹. As such, in certain embodiments, T², if present, is attached to W¹, or V², if present, is attached to W¹. In certain embodiments, L³, if present, is attached to W¹. As such, in certain embodiments, T³, if present, is attached to W¹, or V³, if present, is attached to W¹. In certain embodiments, L⁴, if present, is attached to W¹. As such, in certain embodiments, T⁴, if present, is attached to W¹, or V⁴, if present, is attached to W¹.

Regarding the tether groups, T¹, T², T³ and T⁴, any convenient tether groups may be utilized in the subject linkers. In some embodiments, T¹, T², T³ and T⁴ each comprise one or more groups independently selected from a (C₁-C₁₂)alkyl, a substituted (C₁-C₁₂)alkyl, an (EDA)_{w}, (PEG)ₙ, (AA)ₚ, —(CR¹³OH)ₕ—, piperidin-4-amino (4AP), an acetal group, a disulfide, a hydrazine, and an ester, where w is an integer from 1 to 20, n is an integer from 1 to 30, p is an integer from 1 to 20, and h is an integer from 1 to 12.

In certain embodiments, the tether group (e.g., T¹, T², T³ and/or T⁴) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₂-alkylene.

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₂-alkylene.

In certain embodiments, the tether group (e.g., T¹, T², T³ and/or T⁴) includes an ethylene diamine (EDA) moiety, e.g., an EDA containing tether. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

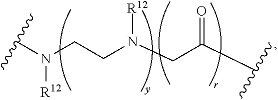

where y is an integer from 1 to 6, r is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group includes a 4-amino-piperidine (4AP) moiety (also referred to as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

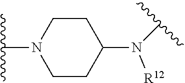

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

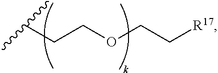

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH.

In certain embodiments, a tether group (e.g., T¹, T², T³ and/or T⁴) includes (PEG)ₙ, where (PEG)ₙ is a polyethylene glycol or a modified polyethylene glycol linking unit. In certain embodiments, (PEG)ₙ is described by the structure:

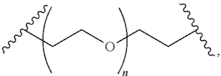

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³ and/or T⁴) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³ and/or T⁴) includes a moiety described by the formula —(CR¹³OH)ₕ—, where h is 0 or n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, h is 1. In certain embodiments, h is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, an alkyl, a substituted alkyl, an aryl, and a substituted aryl. In these embodiments, alkyl, substituted alkyl, aryl, and substituted aryl are as described above for R¹³.

Regarding the linking functional groups, V¹, V², V³ and V⁴, any convenient linking functional groups may be utilized in the subject linkers. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³ and V⁴ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R¹⁵ are described in more detail as follows. In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, the hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl substituents are as described above for R¹⁵.

In certain embodiments, the tether group includes an acetal group, a disulfide, a hydrazine, or an ester. In some embodiments, the tether group includes an acetal group. In some embodiments, the tether group includes a disulfide. In some embodiments, the tether group includes a hydrazine. In some embodiments, the tether group includes an ester.

As described above, in some embodiments, L is a linker comprising -(T¹-V¹)ₐ-(T²-V²)_{b}-(T³-V³)_{c}-(T⁴-V⁴)_{d}—, where a, b, c and d are each independently 0 or 1, where the sum of a, b, c and d is 1 to 4.

In some embodiments, in the subject linker:
T¹ is selected from a (C₁-C₁₂)alkyl and a substituted (C₁-C₁₂)alkyl;
T², T³ and T⁴ are each independently selected from (C₁-C₁₂)alkyl, substituted (C₁-C₁₂)alkyl, (EDA)_{w}, (PEG)ₙ, (AA)ₚ, —(CR¹³OH)ₕ—, 4-amino-piperidine (4AP), an acetal group, a disulfide, a hydrazine, and an ester; and
V¹, V², V³ and V⁴ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
wherein:
(PEG)ₙ is

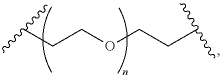

where n is an integer from 1 to 30;
EDA is an ethylene diamine moiety having the following structure:

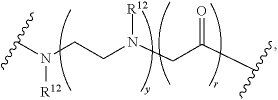

where y is an integer from 1 to 6 and r is 0 or 1;
4-amino-piperidine (4AP) is

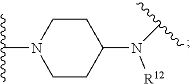

AA is an amino acid residue, where p is an integer from 1 to 20;
each R¹⁵ and R¹² is independently selected from hydrogen, an alkyl, a substituted alkyl, an aryl and a substituted aryl, wherein any two adjacent R¹² groups may be cyclically linked to form a piperazinyl ring; and
R¹³ is selected from hydrogen, an alkyl, a substituted alkyl, an aryl, and a substituted aryl.

In certain embodiments, T¹, T², T³ and T⁴ and V¹, V², V³ and V⁴ are selected from the following table, e.g., one row of the following table (Table A):

TABLE A

| T¹ | V¹ | V² | V² | T³ | V³ | T⁴ | V⁴ |
|---|---|---|---|---|---|---|---|
| (C₁-C₁₂)alkyl | —CONR¹⁵— | (PEG)ₙ | —CO— | — | — | — | — |
| (C₁-C₁₂)alkyl | —CO— | (AA)ₚ | —NR¹⁵— | (PEG)ₙ | —CO— | — | — |
| (C₁-C₁₂)alkyl | —CO— | (AA)ₚ | — | — | — | — | — |
| (C₁-C₁₂)alkyl | —CONR¹⁵— | (PEG)ₙ | —NR¹⁵— | — | — | — | — |
| (C₁-C₁₂)alkyl | —CO— | (AA)ₚ | —NR¹⁵— | (PEG)ₙ | —NR¹⁵— | — | — |
| (C₁-C₁₂)alkyl | —CO— | (EDA)_{w} | —CO— | — | — | — | — |
| (C₁-C₁₂)alkyl | —CONR¹⁵— | (C₁-C₁₂)alkyl | —NR¹⁵— | — | — | — | — |
| (C₁-C₁₂)alkyl | —CONR¹⁵— | (PEG)ₙ | —CO— | (EDA)_{w} | — | — | — |
| (C₁-C₁₂)alkyl | —CO— | (EDA)_{w} | — | — | — | — | — |
| (C₁-C₁₂)alkyl | —CO— | (EDA)_{w} | —CO— | (CR¹³OH)ₕ | —CONR¹⁵— | (C₁-C₁₂)alkyl | —CO— |
| (C₁-C₁₂)alkyl | —CO— | (AA)ₚ | —NR¹⁵— | (C₁-C₁₂)alkyl | —CO— | — | — |
| (C₁-C₁₂)alkyl | —CONR¹⁵— | (PEG)ₙ | —CO— | (AA)ₚ | — | — | — |
| (C₁-C₁₂)alkyl | —CO— | (EDA)_{w} | —CO— | (CR¹³OH)ₕ | —CO— | (AA)ₚ | — |
| (C₁-C₁₂)alkyl | —CO— | (AA)ₚ | —NR¹⁵— | (C₁-C₁₂)alkyl | —CO— | (AA)ₚ | — |
| (C₁-C₁₂)alkyl | —CO— | (AA)ₚ | —NR¹⁵— | (PEG)ₙ | —CO— | (AA)ₚ | — |
| (C₁-C₁₂)alkyl | —CO— | (AA)ₚ | —NR¹⁵— | (PEG)ₙ | —SO₂— | (AA)ₚ | — |
| (C₁-C₁₂)alkyl | —CO— | (EDA)_{w} | —CO— | (CR¹³OH)ₕ | —CONR¹⁵— | (PEG)ₙ | —CO— |
| (C₁-C₁₂)alkyl | —CO— | (CR¹³OH)ₕ | —CO— | — | — | — | — |
| (C₁-C₁₂)alkyl | —CONR¹⁵— | substituted | —NR¹⁵— | (PEG)ₙ | —CO— | — | — |
| | | (C₁-C₁₂)alkyl | | | | | |
| (C₁-C₁₂)alkyl | —SO₂— | (C₁-C₁₂)alkyl | —CO— | — | — | — | — |
| (C₁-C₁₂)alkyl | —CONR¹⁵— | (C₁-C₁₂)alkyl | — | (CR¹³OH)ₕ | —CONR¹⁵— | — | — |
| (C₁-C₁₂)alkyl | —CO— | (AA)ₚ | —NR¹⁵— | (PEG)ₙ | —CO— | (AA)ₚ | —NR¹⁵— |
| (C₁-C₁₂)alkyl | —CO— | (AA)ₚ | —NR¹⁵— | (PEG)ₙ | —P(O)OH— | (AA)ₚ | — |
| (C₁-C₁₂)alkyl | —CO— | (EDA)_{w} | — | (AA)ₚ | — | — | — |
| (C₁-C₁₂)alkyl | —CONR¹⁵— | (C₁-C₁₂)alkyl | —NR¹⁵— | — | —CO— | — | — |
| (C₁-C₁₂)alkyl | —CONR¹⁵— | (C₁-C₁₂)alkyl | —NR¹⁵— | — | —CO— | (C₁-C₁₂)alkyl | —NR¹⁵— |
| (C₁-C₁₂)alkyl | —CO— | 4AP | —CO— | (C₁-C₁₂)alkyl | —CO— | (AA)ₚ | — |
| (C₁-C₁₂)alkyl | —CO— | 4AP | —CO— | (C₁-C₁₂)alkyl | —CO— | — | — |

In certain embodiments, L is a linker comprising -(L¹)ₐ-L²)_{b}-(L³)_{c}(L⁴)_{d}-, where -(L¹)ₐ, is -(T¹-V¹)ₐ, -(L²)_{b}- is -(T²-V²)_{b}—; -(L³)_{c}- is -(T³-V³)_{c}—; and -(L⁴)_{d}- is -(T⁴-V⁴)_{d}—.

In certain embodiments, T¹ is (C₁-C₁₂)alkyl, V¹ is —CO—, T² is (AA)ₚ, V² is —NR¹⁵—, T³ is (PEG)ₙ, V³ is —CO—, T⁴ is absent and V⁴ is absent.

In certain embodiments, T¹ is (C₁-C₁₂)alkyl, V¹ is —CO—, T² is (EDA)_{w}, V² is —CO—, T³ is (CR¹³OH)ₕ, V³ is —CONR¹⁵—, T⁴ is (C₁-C₁₂)alkyl and V⁴ is —CO—.

In certain embodiments, T¹ is (C₁-C₁₂)alkyl, V¹ is —CO—, T² is (AA)ₚ, V² is —NR¹⁵—, T³ is (C₁-C₁₂)alkyl, V³ is —CO—, T⁴ is absent and V⁴ is absent.

In certain embodiments, T¹ is (C₁-C₁₂)alkyl, V¹ is —CONR¹⁵—, T² is (PEG)ₙ, V² is —CO—, T³ is absent, V³ is absent, T⁴ is absent and V⁴ is absent.

In certain embodiments, T¹ is (C₁-C₁₂)alkyl, V¹ is —CO—, T² is (AA)ₚ, V² is absent, T³ is absent, V³ is absent, T⁴ is absent and V⁴ is absent.

In certain embodiments, T¹ is (C₁-C₁₂)alkyl, V¹ is —CONR¹⁵—, T² is (PEG)ₙ, V² is —NR¹⁵—, T³ is absent, V³ is absent, T⁴ is absent and V⁴ is absent.

In certain embodiments, T¹ is (C₁-C₁₂)alkyl, V¹ is —CO—, T² is (AA)ₚ, V² is —NR¹⁵—, T³ is (PEG)ₙ, V³ is —NR¹⁵—, T⁴ is absent and V⁴ is absent.

In certain embodiments, T¹ is (C₁-C₁₂)alkyl, V¹ is —CO—, T² is (EDA)_{w}, V² is —CO—, T³ is absent, V³ is absent, T⁴ is absent and V⁴ is absent.

In certain embodiments, T¹ is (C₁-C₁₂)alkyl, V¹ is —CONR¹⁵—, T² is (C₁-C₁₂)alkyl, V² is —NR¹⁵—, T³ is absent, V³ is absent, T⁴ is absent and V⁴ is absent.

In certain embodiments, T¹ is (C₁-C₁₂)alkyl, V¹ is —CONR¹⁵—, T² is (PEG)ₙ, V² is —CO—, T³ is (EDA)_{w}, V³ is absent, T⁴ is absent and V⁴ is absent.

In certain embodiments, T¹ is (C₁-C₁₂)alkyl, V¹ is —CO—, T² is (EDA)_{w}, V² is absent, T³ is absent, V³ is absent, T⁴ is absent and V⁴ is absent.

In certain embodiments, T¹ is (C₁-C₁₂)alkyl, V¹ is —CONR¹⁵—, T² is (PEG)ₙ, V² is —CO—, T³ is (AA)ₚ, V³ is absent, T⁴ is absent and V⁴ is absent.

In certain embodiments, T¹ is (C₁-C₁₂)alkyl, V¹ is —CO—, T² is (EDA)_{w}, V² is —CO—, T³ is (CR¹³OH)ₕ, V³ is —CO—, T⁴ is (AA)ₚ and V⁴ is absent.

In certain embodiments, T¹ is (C₁-C₁₂)alkyl, V¹ is —CO—, T² is (AA)ₚ, V² is —NR¹⁵—, T³ is (C₁-C₁₂)alkyl, V³ is —CO—, T⁴ is (AA)ₚ and V⁴ is absent.

In certain embodiments, T¹ is (C₁-C₁₂)alkyl, V¹ is —CO—, T² is (AA)ₚ, V² is —NR¹⁵—, T³ is (PEG)ₙ, V³ is —CO—, T⁴ is (AA)ₚ and V⁴ is absent.

In certain embodiments, T¹ is (C₁-C₁₂)alkyl, V¹ is —CO—, T² is (AA)ₚ, V² is —NR¹⁵—, T³ is (PEG)ₙ, V³ is —SO₂—, T⁴ is (AA)ₚ and V⁴ is absent.

In certain embodiments, T¹ is (C₁-C₁₂)alkyl, V¹ is —CO—, T² is (EDA)_{w}, V² is —CO—, T³ is (CR¹³OH)ₕ, V³ is —CONR¹⁵—, T⁴ is (PEG)ₙ and V⁴ is —CO—.

In certain embodiments, T¹ is (C₁-C₁₂)alkyl, V¹ is —CO—, T² is (CR¹³OH)ₕ, V² is —CO—, T³ is absent, V³ is absent, T⁴ is absent and V⁴ is absent.

In certain embodiments, T¹ is (C₁-C₁₂)alkyl, V¹ is —CONR¹⁵—, T² is substituted (C₁-C₁₂)alkyl, V² is —NR¹⁵—, T³ is (PEG)ₙ, V³ is —CO—, T⁴ is absent and V⁴ is absent.

In certain embodiments, T¹ is (C₁-C₁₂)alkyl, V¹ is —SO₂—, T² is (C₁-C₁₂)alkyl, V² is —CO—, T³ is absent, V³ is absent, T⁴ is absent and V⁴ is absent.

In certain embodiments, T¹ is (C₁-C₁₂)alkyl, V¹ is —CONR¹⁵—, T² is (C₁-C₁₂)alkyl, V² is absent, T³ is (CR¹³OH)ₕ, V³ is —CONR¹⁵—, T⁴ is absent and V⁴ is absent.

In certain embodiments, T¹ is (C₁-C₁₂)alkyl, V¹ is —CO—, T² is (AA)ₚ, V² is —NR¹⁵—, T³ is (PEG)ₙ, V³ is —CO—, T⁴ is (AA)ₚ and V⁴ is —NR¹⁵—.

In certain embodiments, T¹ is (C₁-C₁₂)alkyl, V¹ is —CO—, T² is (AA)ₚ, V² is —NR¹⁵—, T³ is (PEG)ₙ, V³ is —P(O)OH—, T⁴ is (AA)ₚ and V⁴ is absent.

In certain embodiments, T¹ is (C₁-C₁₂)alkyl, V¹ is —CO—, T² is (EDA)_{w}, V² is absent, T³ is (AA)ₚ, V³ is absent, T⁴ is absent and V⁴ is absent.

In certain embodiments, T¹ is (C₁-C₁₂)alkyl, V¹ is —CO—, T² is (EDA)_{w}, V² is —CO—, T³ is (CR¹³OH)ₕ, V³ is —CONR¹⁵—, T⁴ is (C₁-C₁₂)alkyl and V⁴ is —CO(AA)ₚ-.

In certain embodiments, T¹ is (C₁-C₁₂)alkyl, V¹ is —CONR¹⁵—, T² is (C₁-C₁₂)alkyl, V² is —NR¹⁵—, T³ is absent, V³ is —CO—, T⁴ is absent and V⁴ is absent.

In certain embodiments, T¹ is (C₁-C₁₂)alkyl, V¹ is —CONR¹⁵—, T² is (C₁-C₁₂)alkyl, V² is —NR¹⁵—, T³ is absent, V³ is —CO—, T⁴ is (C₁-C₁₂)alkyl and V⁴ is —NR¹⁵—.

In certain embodiments, T¹ is (C₁-C₁₂)alkyl, V¹ is —CO—, T² is (EDA)_{w}, V² is —CO—, T³ is (CR¹³OH)ₕ, V³ is —CONR¹⁵—, T⁴ is (PEG)ₙ and V⁴ is —CO(AA)ₚ-.

In certain embodiments, T¹ is (C₁-C₁₂)alkyl, V¹ is —CO—, T² is 4AP, V² is —CO—, T³ is (C₁-C₁₂)alkyl, V³ is —CO—, T⁴ is (AA)ₚ and V⁴ is absent.

In certain embodiments, T¹ is (C₁-C₁₂)alkyl, V¹ is —CO—, T² is 4AP, V² is —CO—, T³ is (C₁-C₁₂)alkyl, V³ is —CO—, T⁴ is absent and V⁴ is absent.

In certain embodiments, the linker is described by one of the following structures:

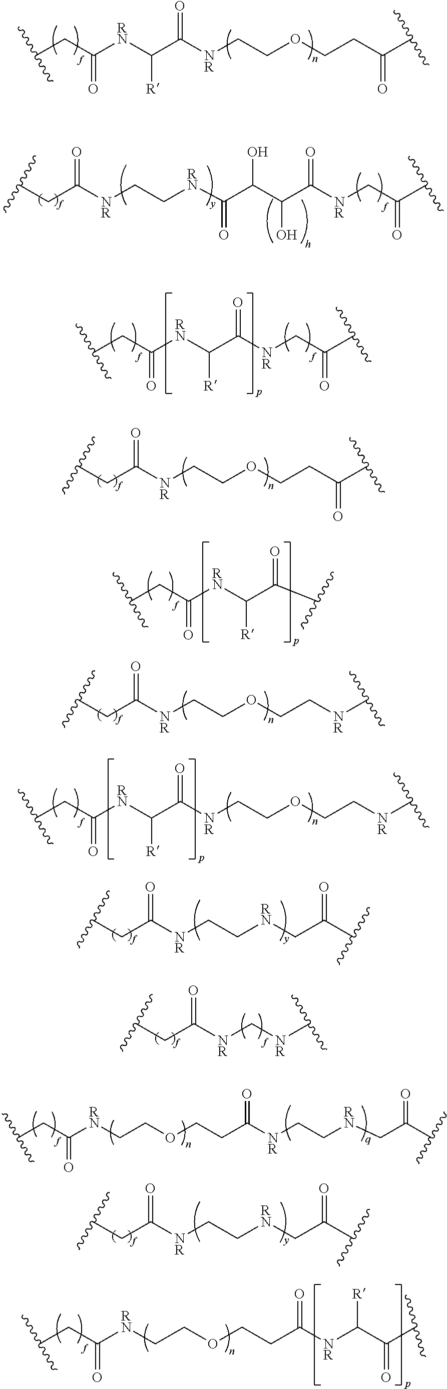

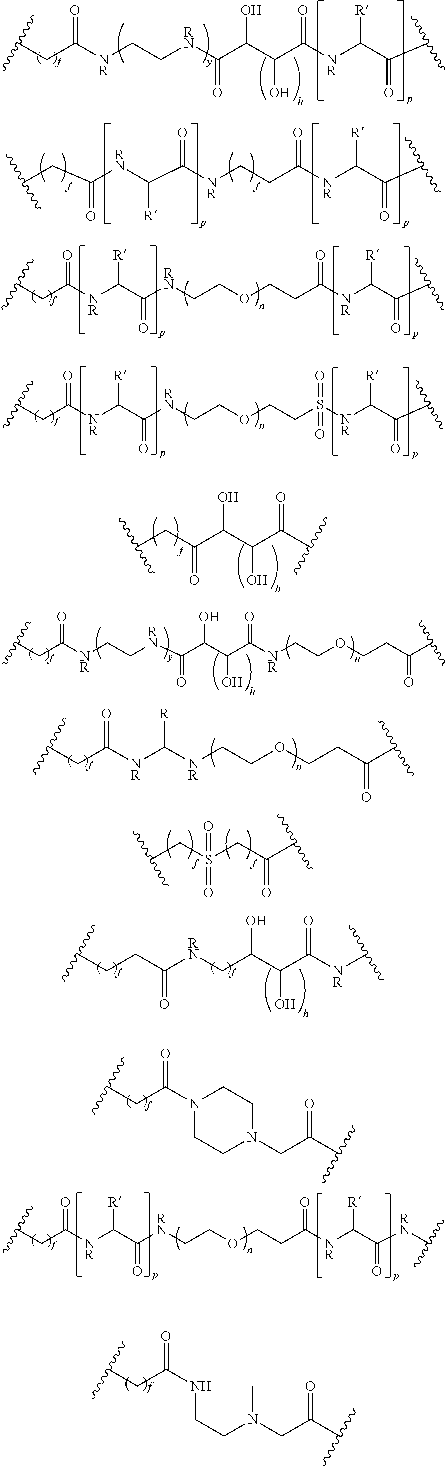

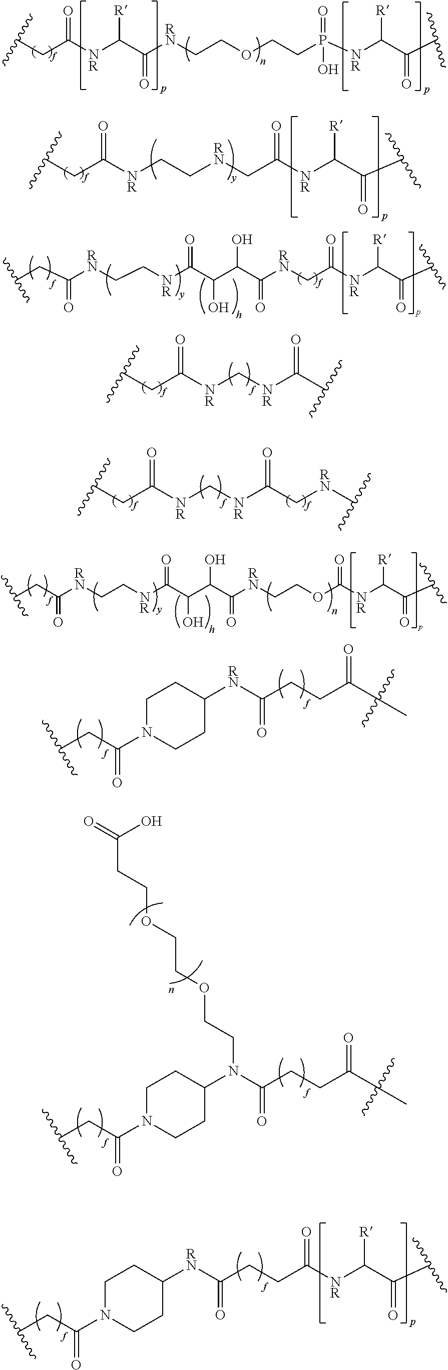

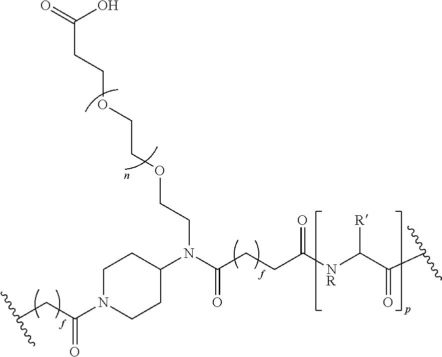

In certain embodiments of the linker structures depicted above, each f is independently 0 or an integer from 1 to 12; each y is independently 0 or an integer from 1 to 20; each n is independently 0 or an integer from 1 to 30; each p is independently 0 or an integer from 1 to 20; each h is independently 0 or an integer from 1 to 12; each R is independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl; and each R′ is independently H, a sidechain of an amino acid, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments of the linker structures depicted above, each f is independently 0, 1, 2, 3, 4, 5 or 6; each y is independently 0, 1, 2, 3, 4, 5 or 6; each n is independently 0, 1, 2, 3, 4, 5 or 6; each p is independently 0, 1, 2, 3, 4, 5 or 6; and each h is independently 0, 1, 2, 3, 4, 5 or 6. In certain embodiments of the linker structures depicted above, each R is independently H, methyl or —(CH₂)ₘ—OH where m is 1, 2, 3 or 4 (e.g., 2).

In certain embodiments of the linker, L, T¹ is (C₁-C₁₂)alkyl, V¹ is —CO—, T² is 4AP, V² is —CO—, T³ is (C₁-C₁₂)alkyl, V³ is —CO—, T⁴ is absent and V⁴ is absent. In certain embodiments, T¹ is ethylene, V¹ is —CO—, T² is 4AP, V² is —CO—, T³ is ethylene, V³ is —CO—, T⁴ is absent and V⁴ is absent. In certain embodiments, T¹ is ethylene, V¹ is —CO—, T² is 4AP, V² is —CO—, T³ is ethylene, V³ is —CO—, T⁴ is absent and V⁴ is absent, where T² (e.g., 4AP) has the following structure:

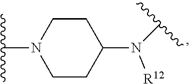

wherein
R¹² is a polyethylene glycol moiety (e.g., a polyethylene glycol or a modified polyethylene glycol).

In certain embodiments, the linker, L, includes the following structure:

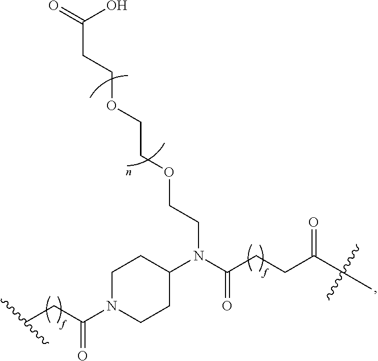

wherein
each f is independently an integer from 1 to 12; and
n is an integer from 1 to 30.

In certain embodiments, f is 1. In certain embodiments, f is 2. In certain embodiments, one f is 2 and one f is 1.

In certain embodiments, n is 1.

In certain embodiments, the left-hand side of the above linker structure is attached to the hydrazinyl-indolyl or the hydrazinyl-pyrrolo-pyridinyl coupling moiety, and the right-hand side of the above linker structure is attached to a drug or active agent.

Any of the chemical entities, linkers and coupling moieties set forth in the structures above may be adapted for use in the subject compounds and conjugates.

Additional disclosure related to hydrazinyl-indolyl and hydrazinyl-pyrrolo-pyridinyl compounds and methods for producing a conjugate is found in U.S. Pat. Nos. 9,310,374, and 9,493,413, the disclosures of each of which are incorporated herein by reference.

### Conjugates of Formula (II)

Aspects of the present disclosure include a conjugate of formula (II):

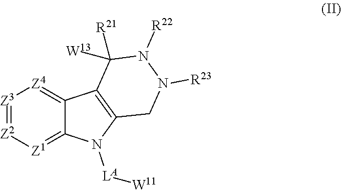

wherein:
Z¹, Z², Z³ and Z⁴ are each independently selected from CR²⁴, N and C-L^{B}-W¹², wherein at least one Z¹, Z², Z³ and Z⁴ is C-L^{B}-W¹²;
R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl;
each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹¹ is a first drug (or active agent);
W¹² is a second drug (or active agent); and
W¹³ is a binding agent as described herein.

The substituents related to conjugates of formula (II) are described in more detail below.

In certain embodiments, Z¹, Z², Z³ and Z⁴ are each independently selected from CR²⁴, N and C-L^{B}-W¹², wherein at least one Z¹, Z², Z³ and Z⁴ is C-L^{B}-W¹². In certain embodiments, Z¹ is CR²⁴. In certain embodiments, Z¹ is N. In certain embodiments, Z¹ is C-L^{B}-W¹². In certain embodiments, Z² is CR²⁴. In certain embodiments, Z² is N. In certain embodiments, Z² is C-L^{B}-W¹². In certain embodiments, Z³ is CR²⁴. In certain embodiments, Z³ is N. In certain embodiments, Z³ is C-L^{B}-W¹². In certain embodiments, Z⁴ is CR²⁴. In certain embodiments, Z⁴ is N. In certain embodiments, Z⁴ is C-L^{B}-W¹².

Combinations of various Z¹, Z², Z³ and Z⁴ are possible. For example, in some instances, Z¹ is C-L^{B}-W¹², Z² is CR²⁴, Z³ is CR²⁴, and Z⁴ is CR²⁴. In some instances, Z¹ is CR²⁴, Z² is C-L^{B}-W¹², Z³ is CR²⁴, and Z⁴ is CR²⁴. In some instances, Z¹ is CR²⁴, Z² is CR²⁴, Z³ is C-L^{B}-W¹², and Z⁴ is CR²⁴. In some instances, Z¹ is CR²⁴, Z² is CR²⁴, Z³ is CR²⁴, and Z⁴ is C-L^{B}-W¹².

In certain embodiments, R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl. In certain embodiments, R²¹ is hydrogen. In certain embodiments, R²¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R²¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl.

In certain embodiments, R²² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R²² is hydrogen. In certain embodiments, R²² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²² is methyl. In certain embodiments, R²² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²² is alkynyl or substituted alkynyl. In certain embodiments, R²² is alkoxy or substituted alkoxy. In certain embodiments, R²² is amino or substituted amino. In certain embodiments, R²² is carboxyl or carboxyl ester. In certain embodiments, R²² is acyl or acyloxy. In certain embodiments, R²² is acyl amino or amino acyl. In certain embodiments, R²² is alkylamide or substituted alkylamide. In certain embodiments, R²² is sulfonyl. In certain embodiments, R²² is thioalkoxy or substituted thioalkoxy. In certain embodiments, R²² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R²² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R²³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R²³ is hydrogen. In certain embodiments, R²³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²³ is methyl. In certain embodiments, R²³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²³ is alkynyl or substituted alkynyl. In certain embodiments, R²³ is alkoxy or substituted alkoxy. In certain embodiments, R²³ is amino or substituted amino. In certain embodiments, R²³ is carboxyl or carboxyl ester. In certain embodiments, R²³ is acyl or acyloxy. In certain embodiments, R²³ is acyl amino or amino acyl. In certain embodiments, R²³ is alkylamide or substituted alkylamide. In certain embodiments, R²³ is sulfonyl. In certain embodiments, R²³ is thioalkoxy or substituted thioalkoxy. In certain embodiments, R²³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R²³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiment, both R²² and R²³ are methyl.

In certain embodiments, R²² and R²³ are optionally cyclically linked to form a 5 or 6-membered heterocyclyl. In certain embodiments, R²² and R²³ are cyclically linked to form a 5 or 6-membered heterocyclyl. In certain embodiments, R²² and R²³ are cyclically linked to form a 5-membered heterocyclyl. In certain embodiments, R²² and R²³ are cyclically linked to form a 6-membered heterocyclyl.

In certain embodiments, each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R²⁴ are described in more detail as follows. In certain embodiments, R²⁴ is hydrogen. In certain embodiments, each R²⁴ is hydrogen. In certain embodiments, R²⁴ is halogen, such as F, Cl, Br or I. In certain embodiments, R²⁴ is F. In certain embodiments, R²⁴ is Cl. In certain embodiments, R²⁴ is Br. In certain embodiments, R²⁴ is I. In certain embodiments, R²⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R²⁴ is methyl. In certain embodiments, R²⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R²⁴ is alkynyl or substituted alkynyl. In certain embodiments, R²⁴ is alkoxy or substituted alkoxy. In certain embodiments, R²⁴ is amino or substituted amino. In certain embodiments, R²⁴ is carboxyl or carboxyl ester. In certain embodiments, R²⁴ is acyl or acyloxy. In certain embodiments, R²⁴ is acyl amino or amino acyl. In certain embodiments, R²⁴ is alkylamide or substituted alkylamide. In certain embodiments, R²⁴ is sulfonyl. In certain embodiments, R²⁴ is thioalkoxy or substituted thioalkoxy. In certain embodiments, R²⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R²⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R²⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R²⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, L^{A} is a first linker. Examples of linkers that can be used in the conjugates of the present disclosure are described in more detail below.

In certain embodiments, L^{B} is a second linker. Examples of linkers that can be used in the conjugates of the present disclosure are described in more detail below.

In certain embodiments, W¹¹ is a first drug (or a first active agent). Examples of drugs and active agents that can be used in the conjugates of the present disclosure are described in more detail below.

In certain embodiments, W¹² is a second drug (or a second active agent). Examples of drugs and active agents that can be used in the conjugates of the present disclosure are described in more detail below.

In certain embodiments, W¹³ is a polypeptide (e.g., an antibody or binding agent as described herein). In certain embodiments, W¹³ comprises one or more fGly′ residues as described herein. In certain embodiments, the polypeptide is attached to the rest of the conjugate through an fGly′ residue as described herein. Examples of polypeptides (e.g., antibodies and binding agents) that can be used in the conjugates of the present disclosure are described in more detail below. In some instances, W¹³ is an antibody (e.g., an antibody as described herein). In some instances, W¹³ is a binding agent (e.g., a binding agent as described herein).

In certain embodiments, the conjugate of formula (II) includes a first linker, L^{A}. The first linker, L^{A}, may be utilized to bind a first moiety of interest (e.g., a first drug or active agent) to a polypeptide (e.g., an antibody) through a conjugation moiety. The first linker, L^{A}, may be bound (e.g., covalently bonded) to the conjugation moiety (e.g., as described herein). For example, the first linker, L^{A}, may attach a hydrazinyl-indolyl or a hydrazinyl-pyrrolo-pyridinyl conjugation moiety to a first drug. The hydrazinyl-indolyl or hydrazinyl-pyrrolo-pyridinyl conjugation moiety may be used to conjugate the first linker, L^{A}, (and thus the first drug) to a polypeptide, such as an antibody.

For example, as shown in formula (II) above, L^{A} is attached to W¹³ through a conjugation moiety, and thus W¹³ is indirectly bonded to the linker L^{A} through the hydrazinyl-indolyl or a hydrazinyl-pyrrolo-pyridinyl conjugation moiety. As described above, W¹³ is a polypeptide (e.g., an antibody or binding agent as described herein), and thus L^{A} is attached through the hydrazinyl-indolyl or a hydrazinyl-pyrrolo-pyridinyl conjugation moiety to the polypeptide (e.g., an antibody or binding agent as described herein), e.g., the linker L^{A} is indirectly bonded to the polypeptide (e.g., an antibody or binding agent as described herein) through the hydrazinyl-indolyl or a hydrazinyl-pyrrolo-pyridinyl conjugation moiety.

Any convenient linker may be utilized for the first linker L^{A} in the subject conjugates and compounds. In certain embodiments, the first linker L^{A} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the first linker L^{A} may include an alkyl or substituted alkyl group. In certain embodiments, the first linker L^{A} may include an alkenyl or substituted alkenyl group. In certain embodiments, the first linker L^{A} may include an alkynyl or substituted alkynyl group. In certain embodiments, the first linker L^{A} may include an alkoxy or substituted alkoxy group. In certain embodiments, the first linker L^{A} may include an amino or substituted amino group. In certain embodiments, the first linker L^{A} may include a carboxyl or carboxyl ester group. In certain embodiments, the first linker L^{A} may include an acyl amino group. In certain embodiments, the first linker L^{A} may include an alkylamide or substituted alkylamide group. In certain embodiments, the first linker L^{A} may include an aryl or substituted aryl group. In certain embodiments, the first linker L^{A} may include a heteroaryl or substituted heteroaryl group. In certain embodiments, the first linker L^{A} may include a cycloalkyl or substituted cycloalkyl group. In certain embodiments, the first linker L^{A} may include a heterocyclyl or substituted heterocyclyl group.

In certain embodiments, the first linker L^{A} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{A} is a first linker described by the formula:

-(L¹)ₐ-(L²)_{b}-(L³)_{c}-(L⁴)_{d}-(L⁵)ₑ-(L⁶)_{f}-,

wherein L¹, L², L³, L⁴, L⁵ and L⁶ are each independently a linker subunit, and a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the hydrazinyl-indolyl or a hydrazinyl-pyrrolo-pyridinyl conjugation moiety (e.g., as shown in formula (I) above). In certain embodiments, the linker subunit L², if present, is attached to the first drug or active agent W¹¹. In certain embodiments, the linker subunit L³, if present, is attached to the first drug or active agent W¹¹. In certain embodiments, the linker subunit L⁴, if present, is attached to the first drug or active agent W¹¹. In certain embodiments, the linker subunit L⁵, if present, is attached to the first drug or active agent W¹¹. In certain embodiments, the linker subunit L⁶, if present, is attached to the first drug or active agent W¹¹.

Any convenient linker subunits may be utilized in the first linker L^{A}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹ (if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵ (if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L^{A} is a first linker comprising -(L¹)ₐ-(L²)_{b}-(L³)_{c}-(L⁴)_{d}-(L⁵)ₑ-(L⁶)_{f}-, where:
-(L¹)ₐ is -(T¹-V¹)ₐ—;
-(L²)_{b}- is -(T²-V²)_{b}—;
-(L³)_{c}- is -(T³-V³)_{c}—;
-(L⁴)_{d}- is -(T⁴-V⁴)_{d}—;
-(L⁵)ₑ- is -(T⁵-V⁵)ₑ—; and
-(L⁶)_{f}- is -(T⁶-V⁶)_{f}—,
wherein T¹, T², T³, T⁴, T⁵ and T⁶, if present, are tether groups;
V¹, V², V³, V⁴, V⁵ and V⁶, if present, are covalent bonds or linking functional groups; and
a, b, c, d, e and f are each independently 0 or 1.

In certain embodiments, the sum of a, b, c, d, e and f is 0 to 6. In certain embodiments, the sum of a, b, c, d, e and f is 0. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

As described above, in certain embodiments, L¹ is attached to the hydrazinyl-indolyl or a hydrazinyl-pyrrolo-pyridinyl conjugation moiety (e.g., as shown in formula (II) above). As such, in certain embodiments, T¹ is attached to the hydrazinyl-indolyl or a hydrazinyl-pyrrolo-pyridinyl conjugation moiety (e.g., as shown in formula (II) above). In certain embodiments, V¹ is attached to the first drug or active agent. In certain embodiments, L², if present, is attached to the first drug or active agent. As such, in certain embodiments, T², if present, is attached to the first drug or active agent, or V², if present, is attached to the first drug or active agent. In certain embodiments, L³, if present, is attached to the first drug or active agent. As such, in certain embodiments, T³, if present, is attached to the first drug or active agent, or V³, if present, is attached to the first drug or active agent. In certain embodiments, L⁴, if present, is attached to the first drug or active agent. As such, in certain embodiments, T⁴, if present, is attached to the first drug or active agent, or V⁴, if present, is attached to the first drug or active agent. In certain embodiments, L⁵, if present, is attached to the first drug or active agent. As such, in certain embodiments, T⁵, if present, is attached to the first drug or active agent, or V⁵, if present, is attached to the first drug or active agent. In certain embodiments, L⁶, if present, is attached to the first drug or active agent. As such, in certain embodiments, T⁶, if present, is attached to the first drug or active agent, or V⁶, if present, is attached to the first drug or active agent.

In certain embodiments, the conjugate of formula (II) includes a second linker, L^{B}. The second linker, L^{B}, may be utilized to bind a second moiety of interest (e.g., a second drug or active agent) to a polypeptide (e.g., an antibody) through a conjugation moiety. The second linker, L^{B}, may be bound (e.g., covalently bonded) to the conjugation moiety (e.g., as described herein). For example, the second linker, L^{B}, may attach a hydrazinyl-indolyl or a hydrazinyl-pyrrolo-pyridinyl conjugation moiety to a second drug. The hydrazinyl-indolyl or hydrazinyl-pyrrolo-pyridinyl conjugation moiety may be used to conjugate the second linker, L^{B}, (and thus the second drug) to a polypeptide, such as an antibody.

For example, as shown in formula (II) above, L^{B} is attached to W¹³ through a conjugation moiety, and thus W¹³ is indirectly bonded to the second linker L^{B} through the hydrazinyl-indolyl or a hydrazinyl-pyrrolo-pyridinyl conjugation moiety. As described above, W¹³ is a polypeptide (e.g., an antibody), and thus L^{B} is attached through the hydrazinyl-indolyl or a hydrazinyl-pyrrolo-pyridinyl conjugation moiety to the polypeptide (antibody), e.g., the linker L^{B} is indirectly bonded to the polypeptide (antibody) through the hydrazinyl-indolyl or a hydrazinyl-pyrrolo-pyridinyl conjugation moiety.

Any convenient linker may be utilized for the second linker L^{B} in the subject conjugates and compounds. In certain embodiments, the second linker L^{B} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the second linker L^{B} may include an alkyl or substituted alkyl group. In certain embodiments, the second linker L^{B} may include an alkenyl or substituted alkenyl group. In certain embodiments, the second linker L^{B} may include an alkynyl or substituted alkynyl group. In certain embodiments, the second linker L^{B} may include an alkoxy or substituted alkoxy group. In certain embodiments, the second linker L^{B} may include an amino or substituted amino group. In certain embodiments, the second linker L^{B} may include a carboxyl or carboxyl ester group. In certain embodiments, the second linker L^{B} may include an acyl amino group. In certain embodiments, the second linker L^{B} may include an alkylamide or substituted alkylamide group. In certain embodiments, the second linker L^{B} may include an aryl or substituted aryl group. In certain embodiments, the second linker L^{B} may include a heteroaryl or substituted heteroaryl group. In certain embodiments, the second linker L^{B} may include a cycloalkyl or substituted cycloalkyl group. In certain embodiments, the second linker L^{B} may include a heterocyclyl or substituted heterocyclyl group.

In certain embodiments, the second linker L^{B} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{B} is a second linker described by the formula:

-(L⁷)g-(L⁸)ₕ-(L⁹)i-(L¹⁰)j-L)ₖ-(L¹²)-(L¹³)ₘ

wherein L⁷, L⁸, L⁹, L¹⁰, L¹¹, L¹² and L¹³ are each independently a linker subunit, and g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and 1 are each 1 and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, l and m are each 0. In certain embodiments, g is 1 and h, i, j, k, l and m are each 0. In certain embodiments, g, h, i, j, k, l and m are each 0.

In certain embodiments, the linker subunit L⁷ is attached to the hydrazinyl-indolyl or a hydrazinyl-pyrrolo-pyridinyl conjugation moiety (e.g., as shown in formula (II) above). In certain embodiments, the linker subunit L⁸, if present, is attached to the second drug or active agent W¹². In certain embodiments, the linker subunit L⁹, if present, is attached to the second drug or active agent W¹². In certain embodiments, the linker subunit L¹⁰, if present, is attached to the second drug or active agent W¹². In certain embodiments, the linker subunit L¹¹, if present, is attached to the second drug or active agent W¹². In certain embodiments, the linker subunit L¹², if present, is attached to the second drug or active agent W¹². In certain embodiments, the linker subunit L¹³, if present, is attached to the second drug or active agent W¹².

Any convenient linker subunits may be utilized in the second linker L^{B}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L⁷, L⁸, L⁹, L¹⁰, L¹¹, L¹² and L¹³ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L⁷ (if present) comprises a polyethylene glycol, a modified polyethylene glycol, an amino acid residue, an alkyl group, a substituted alkyl, an aryl group, a substituted aryl group, or a diamine. In some embodiments, L⁷ comprises a polyethylene glycol. In some embodiments, L⁷ comprises a modified polyethylene glycol. In some embodiments, L⁷ comprises an amino acid residue. In some embodiments, L⁷ comprises an alkyl group or a substituted alkyl. In some embodiments, L⁷ comprises an aryl group or a substituted aryl group. In some embodiments, L⁷ comprises a diamine (e.g., a linking group comprising an alkylene diamine).

In some embodiments, L⁸ (if present) comprises a polyethylene glycol, a modified polyethylene glycol, an amino acid residue, an alkyl group, a substituted alkyl, an aryl group, a substituted aryl group, or a diamine. In some embodiments, L⁸ comprises a polyethylene glycol. In some embodiments, L⁸ comprises a modified polyethylene glycol. In some embodiments, L⁸ comprises an amino acid residue. In some embodiments, L⁸ comprises an alkyl group or a substituted alkyl. In some embodiments, L⁸ comprises an aryl group or a substituted aryl group. In some embodiments, L⁸ comprises a diamine (e.g., a linking group comprising an alkylene diamine).

In some embodiments, L⁹ (if present) comprises a polyethylene glycol, a modified polyethylene glycol, an amino acid residue, an alkyl group, a substituted alkyl, an aryl group, a substituted aryl group, or a diamine. In some embodiments, L⁹ comprises a polyethylene glycol. In some embodiments, L⁹ comprises a modified polyethylene glycol. In some embodiments, L⁹ comprises an amino acid residue. In some embodiments, L⁹ comprises an alkyl group or a substituted alkyl. In some embodiments, L⁹ comprises an aryl group or a substituted aryl group. In some embodiments, L⁹ comprises a diamine (e.g., a linking group comprising an alkylene diamine).

In some embodiments, L¹⁰ (if present) comprises a polyethylene glycol, a modified polyethylene glycol, an amino acid residue, an alkyl group, a substituted alkyl, an aryl group, a substituted aryl group, or a diamine. In some embodiments, L¹⁰ comprises a polyethylene glycol. In some embodiments, L¹⁰ comprises a modified polyethylene glycol. In some embodiments, L¹⁰ comprises an amino acid residue. In some embodiments, L¹⁰ comprises an alkyl group or a substituted alkyl. In some embodiments, L¹⁰ comprises an aryl group or a substituted aryl group. In some embodiments, L¹⁰ comprises a diamine (e.g., a linking group comprising an alkylene diamine).

In some embodiments, L¹¹ (if present) comprises a polyethylene glycol, a modified polyethylene glycol, an amino acid residue, an alkyl group, a substituted alkyl, an aryl group, a substituted aryl group, or a diamine. In some embodiments, L¹¹ comprises a polyethylene glycol. In some embodiments, L¹¹ comprises a modified polyethylene glycol. In some embodiments, L¹¹ comprises an amino acid residue. In some embodiments, L¹¹ comprises an alkyl group or a substituted alkyl. In some embodiments, L¹¹ comprises an aryl group or a substituted aryl group. In some embodiments, L¹¹ comprises a diamine (e.g., a linking group comprising an alkylene diamine).

In some embodiments, L¹² (if present) comprises a polyethylene glycol, a modified polyethylene glycol, an amino acid residue, an alkyl group, a substituted alkyl, an aryl group, a substituted aryl group, or a diamine. In some embodiments, L¹² comprises a polyethylene glycol. In some embodiments, L¹² comprises a modified polyethylene glycol. In some embodiments, L¹² comprises an amino acid residue. In some embodiments, L¹² comprises an alkyl group or a substituted alkyl. In some embodiments, L¹² comprises an aryl group or a substituted aryl group. In some embodiments, L¹² comprises a diamine (e.g., a linking group comprising an alkylene diamine).

In some embodiments, L¹³ (if present) comprises a polyethylene glycol, a modified polyethylene glycol, an amino acid residue, an alkyl group, a substituted alkyl, an aryl group, a substituted aryl group, or a diamine. In some embodiments, L¹³ comprises a polyethylene glycol. In some embodiments, L¹³ comprises a modified polyethylene glycol. In some embodiments, L¹³ comprises an amino acid residue. In some embodiments, L¹³ comprises an alkyl group or a substituted alkyl. In some embodiments, L¹³ comprises an aryl group or a substituted aryl group. In some embodiments, L¹³ comprises a diamine (e.g., a linking group comprising an alkylene diamine).

In some embodiments, L^{B} is a second linker comprising -(L⁷)_{g}-(L⁸)ₕ-(L⁹)ᵢ-(L¹⁰)ⱼ-(L¹¹)ₖ-(L¹²)ₗ-(L¹³)ₘ, where:
-(L⁷)_{g}- is -(T⁷-V⁷)_{g}—;
-(L⁸)ₕ- is -(T⁸-V⁸)ₕ—;
-(L⁹)ᵢ- is -(T⁹-V⁹)ᵢ—;
-(L¹⁰)ⱼ- is -(T¹⁰-V¹⁰)ⱼ—;
-(L¹¹)ₖ- is -(T¹¹-V¹¹)ₖ—;
-(L¹²)₁- is -(T¹²-V¹²)ᵢ—; and
-(L¹³)ₘ is -(T¹³-V¹³)ₘ—,
wherein T⁷, T⁸, T⁹, T¹⁰, T¹¹, T¹² and T¹³, if present, are tether groups;
V⁷, V⁸, V⁹, V¹⁰, V¹¹, V¹² and V¹³, if present, are covalent bonds or linking functional groups; and
g, h, i, j, k, l and m are each independently 0 or 1.

In certain embodiments, the sum of g, h, i, j, k, l and m is 0 to 7. In certain embodiments, the sum of g, h, i, j, k, l and m is 0. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and 1 are each 1 and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, l and m are each 0. In certain embodiments, g is 1 and h, i, j, k, l and m are each 0. In certain embodiments, g, h, i, j, k, l and m are each 0.

As described above, in certain embodiments, L⁷ is attached to the hydrazinyl-indolyl or a hydrazinyl-pyrrolo-pyridinyl conjugation moiety (e.g., as shown in formula (II) above). As such, in certain embodiments, T⁷ is attached to the hydrazinyl-indolyl or a hydrazinyl-pyrrolo-pyridinyl conjugation moiety (e.g., as shown in formula (II) above). In certain embodiments, V⁷ is attached to the second drug or active agent. In certain embodiments, L⁸, if present, is attached to the second drug or active agent. As such, in certain embodiments, T⁸, if present, is attached to the second drug or active agent, or V⁸, if present, is attached to the second drug or active agent. In certain embodiments, L⁹, if present, is attached to the second drug or active agent. As such, in certain embodiments, T⁹, if present, is attached to the second drug or active agent, or V⁹, if present, is attached to the second drug or active agent. In certain embodiments, L¹⁰, if present, is attached to the second drug or active agent. As such, in certain embodiments, T¹⁰, if present, is attached to the second drug or active agent, or V10⁴, if present, is attached to the second drug or active agent. In certain embodiments, L¹¹, if present, is attached to the second drug or active agent. As such, in certain embodiments, T¹¹, if present, is attached to the second drug or active agent, or V¹¹, if present, is attached to the second drug or active agent. In certain embodiments, L¹², if present, is attached to the second drug or active agent. As such, in certain embodiments, T¹², if present, is attached to the second drug or active agent, or V¹², if present, is attached to the second drug or active agent. In certain embodiments, L¹³, if present, is attached to the second drug or active agent. As such, in certain embodiments, T¹³, if present, is attached to the second drug or active agent, or V¹³, if present, is attached to the second drug or active agent.

Regarding the tether groups, T¹, T², T³, T⁴, T⁵, T⁶, T⁷, T⁸, T⁹, T¹⁰, T⁴¹, T¹² and T¹³, any convenient tether groups may be utilized in the subject linkers. In some embodiments, T¹, T², T³, T⁴, T⁵, T⁶, T⁷, T⁸, T⁹, T¹⁰, T¹, T¹² and T¹³ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, where each w is an integer from 1 to 20, each n is an integer from 1 to 30, each p is an integer from 1 to 20, and each x is an integer from 1 to 12.

In certain embodiments, the tether group (e.g., T¹, T², T³, T⁴, T⁵, T⁶, T⁷, T⁸, T⁹, T¹⁰, T¹¹, T¹² and/or T¹³) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₁-alkylene (e.g., CH₂). In some instances, (C₁-C₁₂)alkyl is a C₂-alkylene (e.g., CH₂CH₂). In some instances, (C₁-C₁₂)alkyl is a C₃-alkylene (e.g., CH₂CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₁-alkylene (e.g., C₁-alkylene substituted with —SO₃H). In some instances, substituted (C₁-C₁₂)alkyl is a substituted C₂-alkylene. In some instances, substituted (C₁-C₁₂)alkyl is a substituted C₃-alkylene. For example, substituted (C₁-C₁₂)alkyl may include C₁-C₁₂ alkylene (e.g., C₃-alkylene or C₅-alkylene) substituted with a (PEG)ₖ group as described herein (e.g., —CONH(PEG)ₖ, such as —CONH(PEG)₃ or —CONH(PEG)₅; or —NHCO(PEG)ₖ, such as —NHCO(PEG)₇), or may include C₁-C₁₂ alkylene (e.g., C₃-alkylene) substituted with a —CONHCH₂CH₂SO₃H group, or may include C₁-C₁₂ alkylene (e.g., C₅-alkylene) substituted with a —NHCOCH₂SO₃H group.

In certain embodiments, the tether group (e.g., T¹, T², T³, T⁴, T⁵, T⁶, T⁷, T⁸, T⁹, T¹⁰, T¹¹, T¹² and/or T¹³) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵, T⁶, T⁷, T⁸, T⁹, T¹⁰, T¹¹, T¹² and/or T¹³) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵, T⁶, T⁷, T⁸, T⁹, T¹⁰, T¹¹, T¹² and/or T¹³) includes a heteroaryl or substituted heteroaryl, such triazolyl (e.g., 1,2,3-triazolyl). In some instances, the tether group (e.g., T¹, T², T³, T⁴, T⁵, T⁶, T⁷, T⁸, T⁹, T¹⁰, T⁴, T¹² and/or T¹³) includes a cycloalkyl or substituted cycloalkyl. In some instances, the tether group (e.g., T¹, T², T³, T⁴, T⁵, T⁶, T⁷, T⁸, T⁹, T¹⁰, T¹¹, T¹² and/or T¹³) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵, T⁶, T⁷, T⁸, T⁹, T¹⁰, T¹¹, T¹² and/or T¹³) includes an ethylene diamine (EDA) moiety, e.g., an EDA containing tether group. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

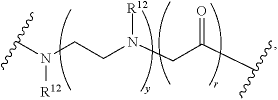

where y is an integer from 1 to 6, or is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T⁵, T⁶, T⁷, T⁸, T⁹, T¹⁰, T¹¹, T¹² and/or T¹³) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

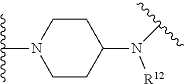

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

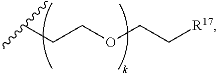

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, OR, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH. In certain embodiments, R¹⁷ is OH. In certain embodiments, R¹⁷ is OCH₃.

In certain embodiments, a tether group (e.g., T¹, T², T³, T⁴, T⁵, T⁶, T⁷, T⁸, T⁹, T¹⁰, T¹¹, T¹² and/or T¹³) includes (PEG)ₙ, where (PEG)ₙ is a polyethylene glycol or a modified polyethyleneglycol linking unit. In certain embodiments, (PEG)ₙ is described by the structure:

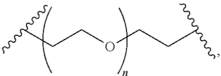

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵, T⁶, T⁷, T⁸, T⁹, T¹⁰, T¹¹, T¹² and/or T¹³) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵, T⁶, T⁷, T⁸, T⁹, T¹⁰, T¹¹, T¹² and/or T¹³) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T¹, T², T³, T⁴, T⁵, T⁶, T⁷, T⁸, T⁹, T¹⁰, T¹¹, T¹² and/or T¹³) includes a moiety described by the formula —(CR¹³OH)ₓ—, where x is 0 or x is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, x is 1. In certain embodiments, x is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³.

In certain embodiments, the tether group (e.g., T¹, T², T³, T⁴, T⁵, T⁶, T⁷, T⁸, T⁹, T¹⁰, T¹¹, T¹² and/or T¹³) includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

In certain embodiments, a tether group (e.g., T¹, T², T³, T⁴, T⁵, T⁶, T⁷, T⁸, T⁹, T¹⁰, T¹¹, T¹² and/or T¹³) includes a meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), or para-hydroxy-phenyl (PHP).

In some embodiments, a tether group includes a MABO group described by the following structure:

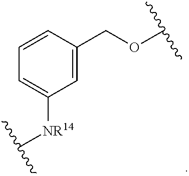

In some embodiments, a tether group includes a MABC group described by the following structure:

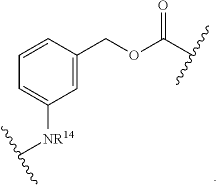

In some embodiments, a tether group includes a PABO group described by the following structure:

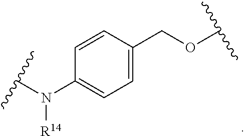

In some embodiments, a tether group includes a PABC group described by the following structure:

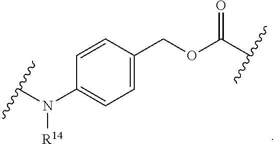

In some embodiments, a tether group includes a PAB group described by the following structure:

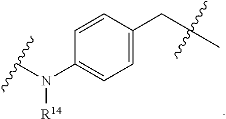

In some embodiments, a tether group includes a PABA group described by the following structure:

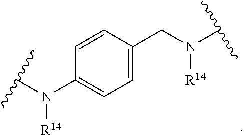

In some embodiments, a tether group includes a PAP group described by the following structure:

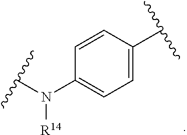

In some embodiments, a tether group includes a PHP group described by the following structure:

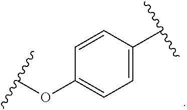 [SMILES: *Oc1ccc(*)cc1]

In certain embodiments, each R¹⁴ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁴ is hydrogen. In certain embodiments, each R¹⁴ is hydrogen. In certain embodiments, R¹⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁴ is alkynyl or substituted alkynyl. In certain embodiments, R¹⁴ is alkoxy or substituted alkoxy. In certain embodiments, R¹⁴ is amino or substituted amino. In certain embodiments, R¹⁴ is carboxyl or carboxyl ester. In certain embodiments, R¹⁴ is acyl or acyloxy. In certain embodiments, R¹⁴ is acyl amino or amino acyl. In certain embodiments, R¹⁴ is alkylamide or substituted alkylamide. In certain embodiments, R¹⁴ is sulfonyl. In certain embodiments, R¹⁴ is thioalkoxy or substituted thioalkoxy. In certain embodiments, R¹⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In some embodiments of the MABO, MABC, PABO, PABC, PAB, PABA, PAP, and PHP tether structures shown above, the phenyl ring may be substituted with one or more additional groups selected from halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, one or more of the tether groups T¹, T², T³, T⁴, T⁵, T⁶, T⁷, T⁸, T⁹, T¹⁰, T¹¹, T¹² and/or T¹³ is each optionally substituted with a glycoside or glycoside derivative. For example, in some instances, T¹, T², T³, T⁴, T⁵ and T⁶ are each optionally substituted with a glycoside. In some instances, T⁷, T⁸, T⁹, T¹⁰, T¹¹, T¹² and T¹³ are each optionally substituted with a glycoside. In certain embodiments, the glycoside or glycoside derivative is selected from a glucuronide, a galactoside, a glucoside, a mannoside, a fucoside, O-GlcNAc, and O-GalNAc.

In certain embodiments, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

For example, in some embodiments, the glycoside or glycoside derivative can be selected from the following structures:

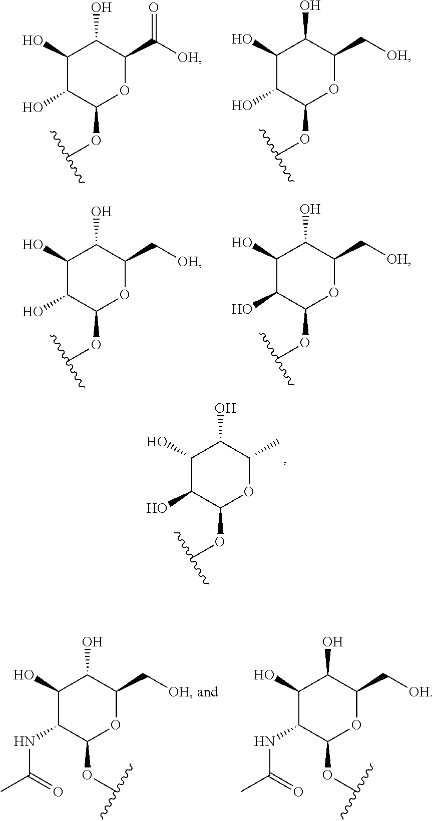

Regarding the linking functional groups, V¹, V², V³, V⁴, V⁵, V⁶, V⁷, V⁸, V⁹, V¹⁰, V¹¹, V¹² and V¹³ any convenient linking functional groups may be utilized in the subject linkers. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵, V⁶, V⁷, V⁸, V⁹, V¹⁰, V¹¹, V¹² and V¹³ are each independently selected from a covalent bond, —CO—, —NR¹⁵, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵.

As described above, in some embodiments, L^{A} is a first linker comprising -(T¹-V¹)ₐ-(T²-V²)_{b}-(T³-V³)_{c}-(T⁴-V⁴)_{d}-(T⁵-V⁵)ₑ-(T⁶-V⁶)_{f}—, where a, b, c, d, e and f are each independently 0 or 1.

In some embodiments, in the first linker L^{A}:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V¹, V², V³, V⁴, V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
   wherein:
(PEG)ₙ is

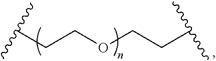

where n is an integer from 1 to 30;
EDA is an ethylene diamine moiety having the following structure:

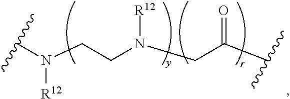

where y is an integer from 1 to 6 and r is 0 or 1;
4-amino-piperidine (4AP) is

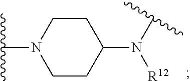

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In certain embodiments, T¹, T², T³, T⁴, T⁵ and T⁶ and V¹, V², V³, V⁴, V⁵ and V⁶ are selected from the following:

wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

In certain embodiments, the left-hand side of the above linker structure for the first linker L^{A} is attached to the hydrazinyl-indolyl or a hydrazinyl-pyrrolo-pyridinyl conjugation moiety, and the right-hand side of the above linker structure for the first linker L^{A} is attached to the first drug or active agent.

As described above, in some embodiments, L^{B} is a second linker comprising -(T⁷-V⁷)_{g}-(T⁸-V⁸)ₕ-(T⁹-V⁹)ᵢ-(T¹⁰-V¹⁰)ⱼ-(T¹¹-V¹¹)ₖ-(T¹²-V¹²)-(T¹³-V¹³)ₘ, where g, h, i, j, k, l and m are each independently 0 or 1.

In some embodiments, in the second linker L^{B}:
T⁷ is selected from a (C₁-C₁₂)alkyl and a substituted (C₁-C₁₂)alkyl;
T⁸, T⁹, T¹⁰, T¹¹, T¹² and T¹³ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PHP, an acetal group, a disulfide, a hydrazine, and an ester; and
V⁷, V⁸, V⁹, V¹⁰, V¹¹, V¹² and V¹³ are each independently selected from a covalent bond, —CO—, —NR¹⁵, —NR¹⁵(CH₂)_{q}—, —NR¹⁵(C₆H₄)—, —CONR¹⁵—, —NR¹⁵CO, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
   wherein:
(PEG)ₙ is

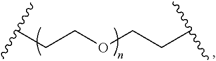

where n is an integer from 1 to 30;
EDA is an ethylene diamine moiety having the following structure:

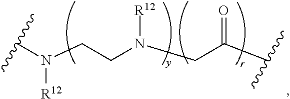

where y is an integer from 1 to 6 and r is 0 or 1;
4-amino-piperidine (4AP) is

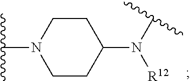

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

Any convenient tether groups may be utilized for T⁷, T⁸, T⁹, T¹⁰, T⁴¹, T¹² and T¹³. For example, any of the tether groups described above in relation to T¹, T², T³, T⁴, T⁵ and T⁶ may be used for the tether groups T⁷, T⁸, T⁹, T¹⁰, T⁴, T¹² and T¹³.

Any convenient linking functional groups may be utilized for V⁷, V⁸, V⁹, V¹⁰, V¹¹, V¹² and V¹³. For example, any of the linking functional groups described above in relation to V¹, V², V³, V⁴, V⁵ and V⁶ may be used for the linking functional groups V⁷, V⁸, V⁹, V¹⁰, V¹¹, V¹² and V³.

In certain embodiments, each R¹³ is independently selected from hydrogen, alkyl, substituted alkyl, aryl, and substituted aryl. In these embodiments, alkyl, substituted alkyl, aryl, and substituted aryl are as described above for R¹³.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵. In these embodiments, various possible substituents are as described above for R¹⁵.

In certain embodiments of the second linker L^{B}, one or more of the tether groups T⁷, T⁸, T⁹, T¹⁰, T⁴, T¹² and T¹³ is each optionally substituted with a glycoside or glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments of the second linker L^{B}, the MABO, MABC, PABO, PABC, PAB, PABA, PAP, and PHP tether structures shown above may be substituted with an one or more additional groups selected from a glycoside and a glycoside derivative. For example, in some embodiments of the MABO, MABC, PABO, PABC, PAB, PABA, PAP, and PHP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments, T⁷, T⁸, T⁹, T¹⁰, T⁴, T¹² and T¹³ and V⁷, V⁸, V⁹, V¹⁰, V¹¹, V¹² and V¹³ are selected from the following:

wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0.

In certain embodiments, the left-hand side of the above linker structure for the second linker L^{B} is attached to the hydrazinyl-indolyl or a hydrazinyl-pyrrolo-pyridinyl conjugation moiety, and the right-hand side of the above linker structure for the second linker L^{B} is attached to the second drug or active agent.

In certain embodiments, the conjugate is an antibody-drug conjugate where the antibody and the drugs are linked together by linkers as described above. In some instances, the linker m (e.g., L^{A} and/or L^{B}) is a cleavable linker. A cleavable linker is a linker that includes one or more cleavable moieties, where the cleavable moiety includes one or more bonds that can dissociate under certain conditions, thus separating the cleavable linker into two or more separable portions. For example, the cleavable moiety may include one or more covalent bonds, which under certain conditions, can dissociate or break apart to separate the cleavable linker into two or more portions. As such the linkers that are included in an antibody-drug conjugate can be cleavable linkers, such that under appropriate conditions, the cleavable linker is cleaved to separate or release the drug from the antibody at a desired target site of action for the drug. In some instances, a cleavable linker includes two cleavable moieties, such as a first cleavable moiety and a second cleavable moiety. The cleavable moieties can be configured such that cleavage of both cleavable moieties is needed in order to separate or release the drug from the antibody at a desired target site of action for the drug. For example, cleavage of a cleavable linker can be achieved by initially cleaving one of the two cleavable moieties and then cleaving the other of the two cleavable moieties. In certain embodiments, a cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (i.e., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, an ester, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as an esterase that cleaves ester bonds or a glycosidase that cleaves glycosidic bonds, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (i.e., the site of the cancer (and overexpressed enzyme)). Thus, in some embodiments, the enzymatically cleavable moiety is a cleavable moiety (e.g., an ester or a glycoside) that can be cleaved by an enzyme that is overexpressed in cancer cells. For instance, the enzyme can be an esterase. As such, in some instances, the enzymatically cleavable moiety is a cleavable moiety (e.g., an ester) that can be cleaved by an esterase enzyme. In some instances, the enzyme can be a glycosidase. As such, in some instances, the enzymatically cleavable moiety is a cleavable moiety (e.g., a glycoside or glycoside derivative) that can be cleaved by a glycosidase enzyme.

In certain embodiments, the enzymatically cleavable moiety is an ester bond. For example, the first cleavable moiety described above (i.e., the cleavable moiety protected from premature cleavage by the second cleavable moiety) can include an ester. The presence of uncleaved second cleavable moiety can protect the first cleavable moiety (ester) from cleavage by an esterase enzyme, and thus substantially reduce or prevent premature release of the drug from the antibody until the antibody-drug conjugate is at or near the desired target site of action for the drug. In some instances, a portion of the linker adjacent to the first cleavable moiety is linked to or includes a substituent, where the substituent comprises the second cleavable moiety. In some instances, the second cleavable moiety includes a glycoside or glycoside derivative.

In some embodiments, the enzymatically cleavable moiety is sugar moiety, such as a glycoside (or glyosyl) or glycoside derivative. In some cases, the glycoside or glycoside derivative can facilitate an increase in the hydrophilicity of the cleavable linker as compared to a cleavable linker that does not include the glycoside or glycoside derivative. The glycoside or glycoside derivative can be any glycoside or glycoside derivative suitable for use in the cleavable linker and that can be cleaved through the enzymatic action of an enzyme. For example, the second cleavable moiety (i.e., the cleavable moiety that protects the first cleavable moiety from premature cleavage) can be a glycoside or glycoside derivative. For instance, in some embodiments, the first cleavable moiety includes an ester and the second cleavable moiety includes a glycoside or glycoside derivative. In certain embodiments, the second cleavable moiety is a glycoside or glycoside derivative selected from a glucuronide, a galactoside, a glucoside, a mannoside, a fucoside, O-GlcNAc, and O-GalNAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is O-GlcNAc. In some instances, the second cleavable moiety is O-GalNAc.

The glycoside or glycoside derivative can be attached (covalently bonded) to the cleavable linker through a glycosidic bond. The glycosidic bond can link the glycoside or glycoside derivative to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some instances, the glycosidic bond is an O-glycosidic bond (an O-glycoside). In some cases, the glycoside or glycoside derivative can be cleaved from the cleavable linker it is attached to by an enzyme (e.g., through enzymatically-mediated hydrolysis of the glycosidic bond). A glycoside or glycoside derivative can be removed or cleaved from the cleavable linker by any convenient enzyme that is able to carry out the cleavage (hydrolysis) of the glycosidic bond that attaches the glycoside or glycoside derivative to the cleavable linker. An example of an enzyme that can be used to mediate the cleavage (hydrolysis) of the glycosidic bond that attaches the glycoside or glycoside derivative to the cleavable linker is a glycosidase, such as a glucuronidase, a galactosidase, a glucosidase, a mannosidase, a fucosidase, and the like. Other suitable enzymes may also be used to mediate the cleavage (hydrolysis) of the glycosidic bond that attaches the glycoside or glycoside derivative to the cleavable linker. In some cases, the enzyme used to mediate the cleavage (hydrolysis) of the glycosidic bond that attaches the glycoside or glycoside derivative to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

Examples of conjugates according to the present disclosure include, but are not limited to, the following structure:

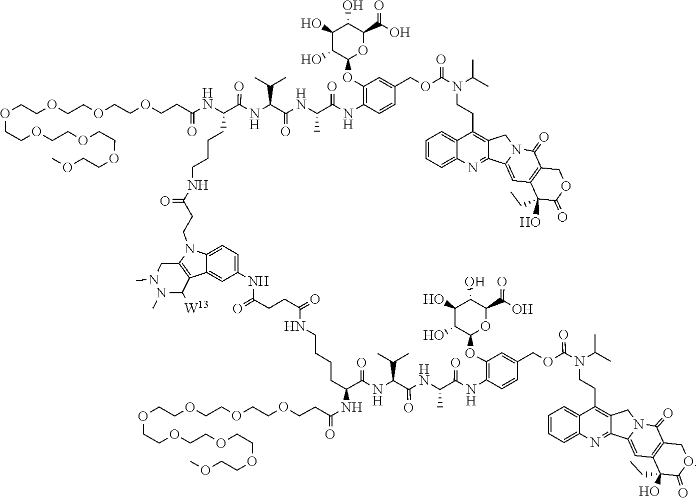

Any of the chemical entities, linkers and conjugation moieties set forth in the structures above may be adapted for use in the subject compounds and conjugates.

Additional disclosure related to hydrazinyl-indolyl and hydrazinyl-pyrrolo-pyridinyl compounds and methods for producing a conjugate is found in U.S. Pat. Nos. 9,310,374 and 9,493,413, the disclosures of each of which are incorporated herein by reference.

### Additional Embodiments of Binding Agent Conjugates

In some cases, the binding agent has an attached moiety of interest, e.g., a detectable label, drug, or half-life-extending moiety. Modification of binding agents can be accomplished by a variety of synthetic and/or recombinant methods. The moiety or moieties attached to a binding agent can provide for one or more of a wide variety of functions or features. Exemplary moieties include detectable labels (e.g., dye labels (e.g., chromophores, fluorophores), biophysical probes (spin labels, nuclear magnetic resonance (NMR) probes), fluorescence Resonance Energy Transfer (FRET)-type labels (e.g., at least one member of a FRET pair, including at least one member of a fluorophore/quencher pair), Bioluminescence Resonance Energy Transfer (BRET)-type labels (e.g., at least one member of a BRET pair), immunodetectable tags (e.g., FLAG, His(6), and the like).

The attached moiety can also be a water soluble polymer (e.g., PEGylation); purification tags (e.g., to facilitate isolation by affinity chromatography (e.g., attachment of a FLAG epitope; membrane localization domains (e.g., lipids or glycophosphatidylinositol (GPI)-type anchors); immobilization tags (e.g., to facilitate attachment of the polypeptide to a surface, including selective attachment); drugs (e.g., to facilitate drug targeting, e.g., through attachment of the drug to an antibody); and the like.

For half-life extension, the binding agents of the present disclosure can optionally be modified to provide for improved pharmacokinetic profile (e.g., by PEGylation, hyperglycosylation, and the like).

In some cases, the binding agent can have a covalently linked non-peptide polymer. Suitable polymers include, e.g., biocompatible polymers, and water-soluble biocompatible polymers. Suitable polymers include synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g. a homo- or hetero-polysaccharide. Suitable polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

Suitable polymers can have an average molecular weight in a range of from 500 Da to 50000 Da, e.g., from 5000 Da to 40000 Da, or from 25000 to 40000 Da. For example, in some embodiments, where a subject antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

In some embodiments, the subject binding agent is covalently linked to a PEG polymer. In some embodiments, a subject scFv multimer is covalently linked to a PEG polymer. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject antibody can be linear or branched. Branched PEG derivatives include star-PEG's and multi-armed PEG's.

A subject binding agent, such as an antibody can be glycosylated, e.g., a subject antibody can comprise a covalently linked carbohydrate or polysaccharide moiety. Glycosylation of antibodies is typically either N-linked or O-linked. Addition of glycosylation sites to an antibody is conveniently accomplished by altering the amino acid sequence such that it contains N- or O-linked glycosylation sites. Similarly, removal of glycosylation sites can be accomplished by amino acid alteration within the native glycosylation sites of an antibody.

A subject binding agent, such as an antibody can be covalently linked to a second moiety (e.g., a lipid, a polypeptide other than a subject antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propionamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

A subject binding agent, such as an antibody can be immobilized on a solid support. Suitable supports are well known in the art and comprise, inter alia, commercially available column materials, polystyrene beads, latex beads, magnetic beads, colloid metal particles, glass and/or silicon chips and surfaces, nitrocellulose strips, nylon membranes, sheets, duracytes, wells of reaction trays (e.g., multi-well plates), plastic tubes, etc. A solid support can comprise any of a variety of substances, including, e.g., glass, polystyrene, polyvinyl chloride, polypropylene, polyethylene, polycarbonate, dextran, nylon, amylose, natural and modified celluloses, polyacrylamides, agaroses, and magnetite. Suitable methods for immobilizing a subject antibody onto a solid support are well known and include, but are not limited to ionic, hydrophobic, covalent interactions and the like. Solid supports can be soluble or insoluble, e.g., in aqueous solution. In some embodiments, a suitable solid support is generally insoluble in an aqueous solution.

A subject binding agent, such as an antibody can in some embodiments comprise a detectable label. Suitable detectable labels include any composition detectable by spectroscopic, photochemical, biochemical, immunochemical, electrical, optical or chemical means. Suitable include, but are not limited to, magnetic beads (e.g. Dynabeads™), fluorescent dyes (e.g., fluorescein isothiocyanate, texas red, rhodamine, a green fluorescent protein, a red fluorescent protein, a yellow fluorescent protein, and the like), radiolabels (e.g., ³H, ¹²⁵, ³⁵S, ¹⁴C, or ²P), enzymes (e.g., horse radish peroxidase, alkaline phosphatase, luciferase, and others commonly used in an enzyme-linked immunosorbent assay (ELISA)), and colorimetric labels such as colloidal gold or colored glass or plastic (e.g. polystyrene, polypropylene, latex, etc.) beads.

In some embodiments, a subject binding agent, such as an antibody comprises a contrast agent or a radioisotope, where the contrast agent or radioisotope is one that is suitable for use as a detectable label, e.g., in imaging, e.g., imaging procedures carried out on humans. Non-limiting examples of labels include radioisotope such as ¹²³¹I (iodine), ¹⁸F (fluorine), ⁹⁹Tc (technetium), ¹¹¹In (indium), and ⁶⁷Ga (gallium), and contrast agent such as gadolinium (Gd), dysprosium, and iron. Radioactive Gd isotopes (¹⁵³Gd) also are available and suitable for imaging procedures in non-human mammals.

A subject binding agent, such as an antibody can be labeled using standard techniques. For example, a subject antibody can be iodinated using chloramine T or 1,3,4,6-tetrachloro-3α,6α-dephenylglycouril. A subject binding agent, such as an antibody can also be labeled with a contrast agent through standard techniques. For example, a subject antibody can be labeled with Gd by conjugating low molecular Gd chelates such as Gd diethylene triamine pentaacetic acid (GdDTPA) or Gd tetraazacyclododecanetetraacetic (GdDOTA) to the antibody. A subject antibody can be labeled with Gd by, for example, conjugating polylysine-Gd chelates to the antibody. Alternatively, a subject antibody can be labeled with Gd by incubating paramagnetic polymerized liposomes that include Gd chelator lipid with avidin and biotinylated antibody.

Suitable fluorescent proteins that can be linked to a subject binding agent include, but are not limited to, a green fluorescent protein from Aequoria victoria or a mutant or derivative thereof, Enhanced GFP, many such GFP which are available commercially, e.g., from Clontech, Inc.; a red fluorescent protein; a yellow fluorescent protein; any of a variety of fluorescent and colored proteins from Anthozoan species; and the like.

A subject binding agent, such as an antibody will in some embodiments comprise a “radiopaque” label, e.g. a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives, radiopaque multiurethanes, organobismuth composites, radiopaque barium multimer complexes, and the like.

A subject binding agent, such as an antibody will in some embodiments be linked to (e.g., covalently or non-covalently linked) a fusion partner, e.g., a ligand; an epitope tag; a peptide; a protein other than an antibody; and the like. Suitable fusion partners include peptides and polypeptides that confer enhanced stability in vivo (e.g., enhanced serum half-life); provide ease of purification, and the like; provide for secretion of the fusion protein from a cell; provide an epitope tag, e.g., His5 (HHHHH) (SEQ ID NO: 177), His X6 (HHHHHH) (SEQ ID NO: 178), C-myc (EQKLISEEDL) (SEQ ID NO: 179), Flag (DYKDDDDK) (SEQ ID NO: 180), StrepTag (WSHPQFEK) (SEQ ID NO: 181), hemagglutinin, e.g., HA Tag (YPYDVPDYA; SEQ ID NO: 182), glutathinone-S-transferase (GST), thioredoxin, cellulose binding domain, RYIRS (SEQ ID NO: 183), Phe-His-His-Thr (SEQ ID NO: 184), chitin binding domain, S-peptide, T⁷ peptide, SH2 domain, C-end RNA tag, WEAAAREACCRECCARA (SEQ ID NO: 185), and the like; provide a detectable signal, e.g., an enzyme that generates a detectable product (e.g., β-galactosidase, luciferase), or a protein that is itself detectable, e.g., a green fluorescent protein, a red fluorescent protein, a yellow fluorescent protein, etc.; provides for multimerization, e.g., a multimerization domain such as an Fc portion of an immunoglobulin; and the like.

In some embodiments, a subject antibody comprises a polyamine modification. A subject antibody can be modified with polyamines that are either naturally occurring or synthetic. Useful naturally occurring polyamines include putrescine, spermidine, spermine, 1,3-deaminopropane, norspermidine, syn-homospermidine, thermine, thermospermine, caldopentamine, homocaldopentamine, and canavalmine. Putrescine, spermidine and spermine are particularly useful. Synthetic polyamines are composed of the empirical formula CxH_{y}Nz, can be cyclic or acyclic, branched or unbranched, hydrocarbon chains of 3-12 carbon atoms that further include 1-6 NR or N(R)₂ moieties, wherein R is H, (C₁-C₄) alkyl, phenyl, or benzyl. Polyamines can be linked to an antibody using any standard crosslinking method.

Where a binding agent, such as an antibody of the present disclosure comprises a covalently linked heterologous moiety, the heterologous moiety can be linked to the heavy and/or light chain directly or via a linker. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Examples of flexible linkers include glycine polymers (G)ₙ, glycine-serine polymers (including, for example, (GS)ₙ, (GSGGS)ₙ (SEQ ID NO: 186) and (GGGS)ₙ (SEQ ID NO: 187), where n is an integer of at least one), glycine-alanine polymers, alanine-serine polymers, and other flexible linkers known in the art.

In some cases, the binding agent, for example, an antibody comprises a covalently linked lipid or fatty acid moiety.

In certain embodiments, the binding agent is conjugated to the agent via a cleavable or a non-cleavable linker. Linkers suitable for use a subject antibody include “flexible linkers.” If present, the linker molecules are generally of sufficient length to permit some flexible movement between linked regions. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like.

### Methods for Modification of Binding Agents

The binding agents, such as antibodies can be modified to have a covalently attached heterologous moiety (e.g., detectable label, drug, etc.) by use of any of a variety of methods. The present disclosure provides a binding agent conjugated to a moiety of interest, where a binding agent conjugated to a moiety of interest is referred to as a “binding agent conjugate.” A binding agent conjugate of the present disclosure can include: 1) Ig heavy chain constant region conjugated to a moiety of interest; and an Ig light chain constant region conjugated to a moiety of interest; 2) an Ig heavy chain constant region conjugated to a moiety of interest; and an Ig light chain constant region that is not conjugated to a moiety of interest; or 3) an Ig heavy chain constant region that is not conjugated to a moiety of interest; and an Ig light chain constant region conjugated to a moiety of interest. A subject binding agent conjugate can include VH and/or VL domains as described herein.

As described above, the binding agent can be modified to include a 2-formylglycine residue (fGly), which can serve as a chemical handle for attachment of a heterologous moiety. For example, the heavy and/or light chain constant region of a binding agent of the present disclosure can be modified to include an amino acid sequence of a sulfatase motif which is capable of being converted by action of a 2-formylglycine generating enzyme (FGE) to contain a 2-formylglycine (fGly). Such sulfatase motifs may also be referred to herein as an FGE-modification site. Action of FGE is directed in a sequence-specific manner in that the FGE acts at a sulfatase motif positioned within the immunoglobulin polypeptide. The moiety of interest is provided as a component of a reactive partner for reaction with an aldehyde of the fGly residue of a converted aldehyde tag of the tagged Ig polypeptide, as described herein.

### Modified Constant Region Sequences

In some cases, the amino acid sequence of a binding agent disclosed herein, for example, an antibody is modified to include a sulfatase motif that contains a serine or cysteine residue that is capable of being converted (oxidized) to a 2-formylglycine (fGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8 or 7 amino acid residues in length.

In certain embodiments, binding agents of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the binding agents. In certain embodiments, the polypeptide includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the binding agents. Where an amino acid sequence native to the binding agent (e.g., anti-CD30 antibody) contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif. In certain embodiments, the extent of modification of the native amino acid sequence of the target binding agent, such as anti-CD30 antibody, is minimized, so as to minimize the number of amino acid residues that are inserted, deleted, substituted (replaced), or added (e.g., to the N- or C-terminus). Minimizing the extent of amino acid sequence modification of the binding agents, such as anti-CD30 antibodies, may minimize the impact such modifications may have upon the function and/or structure of the binding agents.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH1 domain of an Ig heavy chain. An aldehyde tag can be present within a CH2 domain of an Ig heavy chain. An aldehyde tag can be present within a CH3 domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

X¹Z¹⁰X²Z²⁰X³Z³⁰ (I′)

where
Z¹⁰ is cysteine or serine (which can also be represented by (C/S));
Z²⁰ is either a proline or alanine residue (which can also be represented by (P/A));
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I;
X¹ is present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M, S or V, with the proviso that when the sulfatase motif is at the N-terminus of the binding agent, X¹ is present; and
X² and X³ independently can be any amino acid, though usually an aliphatic amino acid, a polar, uncharged amino acid, or a sulfur containing amino acid (i.e., other than an aromatic amino acid or a charged amino acid), e.g., S, T, A, V, G or C, e.g., S, T, A, V or G.

The amino acid sequence of an antibody heavy and/or light chain can be modified to provide a sequence of at least 5 amino acids of the formula X¹Z¹⁰X²Z²⁰X³Z³⁰, where
Z¹⁰ is cysteine or serine;
Z²⁰ is a proline or alanine residue;
Z³⁰ is an aliphatic amino acid or a basic amino acid;
X¹ is present or absent and, when present, is any amino acid, with the proviso that when the heterologous sulfatase motif is at an N-terminus of the polypeptide, X¹ is present;
X² and X³ are each independently any amino acid,
where the sequence is within or adjacent a solvent-accessible loop region of the Ig constant region, and wherein the sequence is not at the C-terminus of the Ig heavy chain.

The sulfatase motif is generally selected so as to be capable of conversion by a selected FGE, e.g., an FGE present in a host cell in which the aldehyde tagged binding agent is expressed or an FGE which is to be contacted with the aldehyde tagged polypeptide in a cell-free in vitro method.

For example, where the FGE is a eukaryotic FGE (e.g., a mammalian FGE, including a human FGE), the sulfatase motif can be of the formula:

X¹CX²PX³Z³⁰ (I″)

where
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, S or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G, or C, e.g., S, T, A, V or G; and
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include VCTPSR (SEQ ID NO: 37), LCSPSR (SEQ ID NO: 38), LCAPSR (SEQ ID NO: 39), LCVPSR (SEQ ID NO: 40), LCGPSR (SEQ ID NO: 41), ICTPAR (SEQ ID NO: 42), LCTPSK (SEQ ID NO: 43), MCTPSK (SEQ ID NO: 44), VCTPSK (SEQ ID NO: 45), LCSPSK (SEQ ID NO: 46), LCAPSK (SEQ ID NO: 47), LCVPSK (SEQ ID NO: 48), LCGPSK (SEQ ID NO: 49), LCTPSA (SEQ ID NO: 50), ICTPAA (SEQ ID NO: 51), MCTPSA (SEQ ID NO: 52), VCTPSA (SEQ ID NO: 53), LCSPSA (SEQ ID NO: 54), LCAPSA (SEQ ID NO: 55), LCVPSA (SEQ ID NO: 56), LCGPSA (SEQ ID NO: 57), LCTPSR (SEQ ID NO: 58), and MCTPSR (SEQ ID NO: 59).

fGly-Containing Sequences

Upon action of FGE on the heavy and/or light chain, the serine or the cysteine in the sulfatase motif is converted to fGly. Thus, the fGly-containing sulfatase motif can be of the formula:

X¹(fGly)X²Z²⁰X³Z³⁰ (I″′)

where
fGly is the formylglycine residue;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

As described above, to produce the conjugate, the binding agent containing the fGly residue may be conjugated to a drug or active agent by reaction of the fGly with a reactive moiety (e.g., hydrazinyl-indolyl or a hydrazinyl-pyrrolo-pyridinyl coupling moiety, as described above) of a linker attached to the drug or active agent to produce an fGly′-containing sulfatase motif. As used herein, the term fGly′ refers to the amino acid residue of the sulfatase motif that is coupled to the drug or active agent through a linker as described herein. Thus, the fGly′-containing sulfatase motif can be of the formula:

X¹(fGly′)X²Z²⁰X³Z³⁰ (II)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the binding agent, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

In certain embodiments, the amino acid residue coupled to the drug or active agent is positioned at a C-terminus of a heavy chain constant region of the binding agent. In some instances, the heavy chain constant region comprises a sequence of the formula (III):

X¹(fGly′)X²Z²⁰X³Z³⁰ (III)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and
wherein the sequence is C-terminal to the amino acid sequence QKSLSLSPGK (SEQ ID NO: 188), and where the sequence may include 1, 2, 3, 4, 5, or from 5 to 10, amino acids that are not present in a native, wild-type heavy Ig chain constant region.

In certain embodiments, the heavy chain constant region comprises the sequence SLSLSPGSL(fGly′)TPSRGS (SEQ ID NO: 60) at the C-terminus of the Ig heavy chain, e.g., in place of a native SLSLSPGK (SEQ ID NO: 61) sequence.

In certain embodiments, the amino acid residue coupled to the drug or active agent is positioned in a light chain constant region of the binding agent. In certain embodiments, the light chain constant region comprises a sequence of the formula (III):

X¹(fGly′)X²Z²⁰X³Z³⁰ (III)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and wherein the sequence is C-terminal to the amino acid sequence KVDNAL (SEQ ID NO: 62) and/or is N-terminal to the amino acid sequence QSGNSQ (SEQ ID NO: 63).

In certain embodiments, the light chain constant region comprises the sequence KVDNAL(fGly′)TPSRQSGNSQ (SEQ ID NO: 64).

In certain embodiments, the amino acid residue coupled to the drug or active agent is positioned in a heavy chain CH1 region of a binding agent, such as an antibody. In certain embodiments, the heavy chain CH1 region comprises a sequence of the formula (III):

X¹(fGly′)X²Z²⁰X³Z³⁰ (III)

where
fGly′ is the amino acid residue coupled to the drug or active agent through a linker as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target polypeptide, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G; and
wherein the sequence is C-terminal to the amino acid sequence SWNSGA (SEQ ID NO: 65) and/or is N-terminal to the amino acid sequence GVHTFP (SEQ ID NO: 66).

In certain embodiments, the heavy chain CH1 region comprises the sequence SWNSGAL(fGly′)TPSRGVHTFP (SEQ ID NO: 67).

### Site of Modification

As noted above, the amino acid sequence of a binding agent, such as an antibody is modified to include a sulfatase motif that contains a serine or cysteine residue that is capable of being converted (oxidized) to an fGly residue by action of an FGE either in vivo (e.g., at the time of translation of an aldehyde tag-containing protein in a cell) or in vitro (e.g., by contacting an aldehyde tag-containing protein with an FGE in a cell-free system). The binding agent used to generate a conjugate of the present disclosure include at least an Ig constant region, e.g., an Ig heavy chain constant region (e.g., at least a CH1 domain; at least a CH1 and a CH2 domain; a CH1, a CH2, and a CH3 domain; or a CH1, a CH2, a CH3, and a CH4 domain), or an Ig light chain constant region. Such Ig polypeptides are referred to herein as “target Ig polypeptides” or “target anti-CD30 antibodies” or “target anti-CD30 Ig polypeptides.”

The site in a binding agent, such as an antibody, particularly, anti-CD30 antibody, into which a sulfatase motif is introduced can be any convenient site. As noted above, in some instances, the extent of modification of the native amino acid sequence of the target anti-CD30 polypeptide is minimized, so as to minimize the number of amino acid residues that are inserted, deleted, substituted (replaced), and/or added (e.g., to the N- or C-terminus). Minimizing the extent of amino acid sequence modification of the target anti-CD30 polypeptide may minimize the impact such modifications may have upon anti-CD30 function and/or structure.

In an antibody, such as an anti-CD30 antibody, heavy chain constant region can include Ig constant regions of any heavy chain isotype, non-naturally occurring Ig heavy chain constant regions (including consensus Ig heavy chain constant regions). An Ig constant region amino acid sequence can be modified to include an aldehyde tag, where the aldehyde tag is present in or adjacent a solvent-accessible loop region of the Ig constant region. An Ig constant region amino acid sequence can be modified by insertion and/or substitution of 1, 2, 3, 4, 5, 6, 7, 8, 9, 10, 11, 12, 13, 14, 15, or 16 amino acids, or more than 16 amino acids, to provide an amino acid sequence of a sulfatase motif as described above.

In some cases, an aldehyde-tagged antibody, such as anti-CD30 antibody comprises an aldehyde-tagged Ig heavy chain constant region (e.g., at least a CH1 domain; at least a CH1 and a CH2 domain; a CH1, a CH2, and a CH3 domain; or a CH1, a CH2, a CH3, and a CH4 domain). The aldehyde-tagged Ig heavy chain constant region can include heavy chain constant region sequences of an IgA, IgM, IgD, IgE, IgG1, IgG2, IgG3, or IgG4 isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, etc., that includes at least one sulfatase motif that can be modified by an FGE to generate an fGly-modified Ig polypeptide. Allotypic variants of Ig heavy chains are known in the art. See, e.g., Jefferis and Lefranc (2009) MAbs 1:4.

In some cases, an aldehyde-tagged antibody, such as an anti-CD30 antibody comprises an aldehyde-tagged Ig light chain constant region. The aldehyde-tagged Ig light chain constant region can include constant region sequences of a kappa light chain, a lambda light chain, e.g., human kappa or lambda light chain constant regions, a hybrid light chain constant region, a synthetic light chain constant region, or a consensus light chain constant region sequence, etc., that includes at least one sulfatase motif that can be modified by an FGE to generate an fGly-modified antibody, such as an fGly-modified anti-CD30 antibody. Exemplary constant regions include human gamma 1 and gamma 3 regions. With the exception of the sulfatase motif, a constant region may have a wild-type amino acid sequence, or it may have an amino acid sequence that is at least 70% identical (e.g., at least 80%, at least 90% or at least 95% identical) to a wild type amino acid sequence.

In some embodiments the sulfatase motif is at a position other than, or in addition to, the C-terminus of the Ig polypeptide heavy chain. As noted above, an isolated aldehyde-tagged antibody, such as an anti-CD30 antibody, can comprise a heavy chain constant region amino acid sequence modified to include a sulfatase motif as described above, where the sulfatase motif is in or adjacent to a surface-accessible loop region of the antibody heavy chain constant region.

In some instances, in an antibody, such as an anti-CD30 antibody, immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG1 heavy chain constant region corresponding to one or more of: 1) amino acids 122-127; 2) amino acids 137-143; 3) amino acids 155-158; 4) amino acids 163-170; 5) amino acids 163-183; 6) amino acids 179-183; 7) amino acids 190-192; 8) amino acids 200-202; 9) amino acids 199-202; 10) amino acids 208-212; 11) amino acids 220-241; 12) amino acids 247-251; 13) amino acids 257-261; 14) amino acid 269-277; 15) amino acids 271-277; 16) amino acids 284-285; 17) amino acids 284-292; 18) amino acids 289-291; 19) amino acids 299-303; 20) amino acids 309-313; 21) amino acids 320-322; 22) amino acids 329-335; 23) amino acids 341-349; 24) amino acids 342-348; 25) amino acids 356-365; 26) amino acids 377-381; 27) amino acids 388-394; 28) amino acids 398-407; 29) amino acids 433-451; and 30) amino acids 446-451; wherein the amino acid numbering is based on the amino acid numbering of human IgG1.

In some instances, in an antibody, such as an anti-CD30 antibody, immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG1 heavy chain constant region corresponding to one or more of: 1) amino acids 1-6; 2) amino acids 16-22; 3) amino acids 34-47; 4) amino acids 42-49; 5) amino acids 42-62; 6) amino acids 34-37; 7) amino acids 69-71; 8) amino acids 79-81; 9) amino acids 78-81; 10) amino acids 87-91; 11) amino acids 100-121; 12) amino acids 127-131; 13) amino acids 137-141; 14) amino acid 149-157; 15) amino acids 151-157; 16) amino acids 164-165; 17) amino acids 164-172; 18) amino acids 169-171; 19) amino acids 179-183; 20) amino acids 189-193; 21) amino acids 200-202; 22) amino acids 209-215; 23) amino acids 221-229; 24) amino acids 22-228; 25) amino acids 236-245; 26) amino acids 217-261; 27) amino acids 268-274; 28) amino acids 278-287; 29) amino acids 313-331; and 30) amino acids 324-331; wherein the amino acid numbering is based on the amino acid numbering of human IgG1 (human IgG1 constant region).

Exemplary surface-accessible loop regions of an IgG1 heavy chain include: 1) ASTKGP (SEQ ID NO: 68); 2) KSTSGGT (SEQ ID NO: 69); 3) PEPV (SEQ ID NO: 70); 4) NSGALTSG (SEQ ID NO: 71); 5) NSGALTSGVHTFPAVLQSSGL (SEQ ID NO: 72); 6) QSSGL (SEQ ID NO: 73); 7) VTV; 8) QTY; 9) TQTY (SEQ ID NO: 74); 10) HKPSN (SEQ ID NO: 75); 11) EPKSCDKTHTCPPCPAPELLGG (SEQ ID NO: 76); 12) FPPKP (SEQ ID NO: 77); 13) ISRTP (SEQ ID NO: 78); 14) DVSHEDPEV (SEQ ID NO: 79); 15) SHEDPEV (SEQ ID NO: 80); 16) DG; 17) DGVEVHNAK (SEQ ID NO: 81); 18) HNA; 19) QYNST (SEQ ID NO: 82); 20) VLTVL (SEQ ID NO: 83); 21) GKE; 22) NKALPAP (SEQ ID NO: 84); 23) SKAKGQPRE (SEQ ID NO: 85); 24) KAKGQPR (SEQ ID NO: 86); 25) PPSRKELTKN (SEQ ID NO: 87); 26) YPSDI (SEQ ID NO: 88); 27) NGQPENN (SEQ ID NO: 89); 28) TPPVLDSDGS (SEQ ID NO: 90); 29) HEALHNHYTQKSLSLSPGK (SEQ ID NO: 91); and 30) SLSPGK (SEQ ID NO: 92).

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG2 heavy chain constant region corresponding to one or more of: 1) amino acids 1-6; 2) amino acids 13-24; 3) amino acids 33-37; 4) amino acids 43-54; 5) amino acids 58-63; 6) amino acids 69-71; 7) amino acids 78-80; 8) 87-89; 9) amino acids 95-96; 10) 114-118; 11) 122-126; 12) 134-136; 13) 144-152; 14) 159-167; 15) 175-176; 16) 184-188; 17) 195-197; 18) 204-210; 19) 216-224; 20) 231-233; 21) 237-241; 22) 252-256; 23) 263-269; 24) 273-282; 25) amino acids 299-302; where the amino acid numbering is based on the numbering of the amino acid sequence (human IgG2).

Exemplary surface-accessible loop regions of an IgG2 heavy chain include 1) ASTKGP (SEQ ID NO: 68); 2) PCSRSTSESTAA (SEQ ID NO: 93); 3) FPEPV (SEQ ID NO: 94); 4) SGALTSGVHTFP (SEQ ID NO: 95); 5) QSSGLY (SEQ ID NO: 96); 6) VTV; 7) TQT; 8) HKP; 9) DK; 10) VAGPS (SEQ ID NO: 97); 11) FPPKP (SEQ ID NO: 77); 12) RTP; 13) DVSHEDPEV (SEQ ID NO: 79); 14) DGVEVHNAK (SEQ ID NO: 81); 15) FN; 16) VLTVV (SEQ ID NO: 101); 17) GKE; 18) NKGLPAP (SEQ ID NO: 102); 19) SKTKGQPRE (SEQ ID NO: 103); 20) PPS; 21) MTKNQ (SEQ ID NO: 104); 22) YPSDI (SEQ ID NO: 88); 23) NGQPENN (SEQ ID NO: 89); 24) TPPMLDSDGS (SEQ ID NO: 107); 25) GNVF (SEQ ID NO: 108); and 26) HEALHNHYTQKSLSLSPGK (SEQ ID NO: 91).

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG3 heavy chain constant region corresponding to one or more of: 1) amino acids 1-6; 2) amino acids 13-22; 3) amino acids 33-37; 4) amino acids 43-61; 5) amino acid 71; 6) amino acids 78-80; 7) 87-91; 8) amino acids 97-106; 9) 111-115; 10) 147-167; 11) 173-177; 16) 185-187; 13) 195-203; 14) 210-218; 15) 226-227; 16) 238-239; 17) 246-248; 18) 255-261; 19) 267-275; 20) 282-291; 21) amino acids 303-307; 22) amino acids 313-320; 23) amino acids 324-333; 24) amino acids 350-352; 25) amino acids 359-365; and 26) amino acids 372-377; where the amino acid numbering is based on the numbering of the amino acid sequence (human IgG3).

Exemplary surface-accessible loop regions of an IgG3 heavy chain include 1) ASTKGP (SEQ ID NO: 68); 2) PCSRSTSGGT (SEQ ID NO: 110); 3) FPEPV (SEQ ID NO: 94); 4) SGALTSGVHTFPAVLQSSG (SEQ ID NO: 112); 5) V; 6) TQT; 7) HKPSN (SEQ ID NO: 75); 8) RVELKTPLGD (SEQ ID NO: 114); 9) CPRCPKP (SEQ ID NO: 115); 10) PKSCDTPPPCPRCPAPELLGG (SEQ ID NO: 116); 11) FPPKP (SEQ ID NO: 77); 12) RTP; 13) DVSHEDPEV (SEQ ID NO: 79); 14) DGVEVHNAK (SEQ ID NO: 81); 15) YN; 16) VL; 17) GKE; 18) NKALPAP (SEQ ID NO: 84); 19) SKTKGQPRE (SEQ ID NO: 103); 20) PPSREEMTKN (SEQ ID NO: 122); 21) YPSDI (SEQ ID NO: 88); 22) SSGQPENN (SEQ ID NO: 124); 23) TPPMLDSDGS (SEQ ID NO: 107); 24) GNI; 25) HEALHNR (SEQ ID NO: 126); and 26) SLSPGK (SEQ ID NO: 92).

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an IgG4 heavy chain constant region corresponding to one or more of: 1) amino acids 1-5; 2) amino acids 12-23; 3) amino acids 32-36; 4) amino acids 42-53; 5) amino acids 57-62; 6) amino acids 68-70; 7) amino acids 77-79; 8) amino acids 86-88; 9) amino acids 94-95; 10) amino acids 101-102; 11) amino acids 108-118; 12) amino acids 122-126; 13) amino acids 134-136; 14) amino acids 144-152; 15) amino acids 159-167; 16) amino acids 175-176; 17) amino acids 185-186; 18) amino acids 196-198; 19) amino acids 205-211; 20) amino acids 217-226; 21) amino acids 232-241; 22) amino acids 253-257; 23) amino acids 264-265; 24) 269-270; 25) amino acids 274-283; 26) amino acids 300-303; 27) amino acids 399-417; where the amino acid numbering is based on the numbering of the amino acid sequence (human IgG4).

Exemplary surface-accessible loop regions of an IgG4 heavy chain include 1) STKGP (SEQ ID NO: 128); 2) PCSRSTSESTAA (SEQ ID NO: 93); 3) FPEPV (SEQ ID NO: 94); 4) SGALTSGVHTFP (SEQ ID NO: 95); 5) QSSGLY (SEQ ID NO: 96); 6) VTV; 7) TKT; 8) HKP; 9) DK; 10) YG; 11) CPAPEFLGGPS (SEQ ID NO: 133); 12) FPPKP (SEQ ID NO: 77); 13) RTP; 14) DVSQEDPEV (SEQ ID NO: 135); 15) DGVEVHNAK (SEQ ID NO: 81); 16) FN; 17) VL; 18) GKE; 19) NKGLPSS (SEQ ID NO: 137); 20) SKAKGQPREP (SEQ ID NO: 138); 21) PPSQEEMTKN (SEQ ID NO: 139); 22) YPSDI (SEQ ID NO: 88); 23) NG; 24) NN; 25) TPPVLDSDGS (SEQ ID NO: 90); 26) GNVF (SEQ ID NO: 108); and 27) HEALHNHYTQKSLSLSLGK (SEQ ID NO: 143).

In some instances, a binding agent is modified to include a sulfatase motif as described above, where the modification includes one or more amino acid residue insertions, deletions, and/or substitutions. In certain embodiments, the sulfatase motif is within, or adjacent to, a region of an IgA heavy chain constant region corresponding to one or more of: 1) amino acids 1-13; 2) amino acids 17-21; 3) amino acids 28-32; 4) amino acids 44-54; 5) amino acids 60-66; 6) amino acids 73-76; 7) amino acids 80-82; 8) amino acids 90-91; 9) amino acids 123-125; 10) amino acids 130-133; 11) amino acids 138-142; 12) amino acids 151-158; 13) amino acids 165-174; 14) amino acids 181-184; 15) amino acids 192-195; 16) amino acid 199; 17) amino acids 209-210; 18) amino acids 222-245; 19) amino acids 252-256; 20) amino acids 266-276; 21) amino acids 293-294; 22) amino acids 301-304; 23) amino acids 317-320; 24) amino acids 329-353; where the amino acid numbering is based on the numbering of the amino acid sequence (human IgA).

Exemplary surface-accessible loop regions of an IgA heavy chain include 1) ASPTSPKVFPLSL (SEQ ID NO: 144); 2) QPDGN (SEQ ID NO: 145); 3) VQGFFPQEPL (SEQ ID NO: 146); 4) SGQGVTARNFP (SEQ ID NO: 147); 5) SGDLYTT (SEQ ID NO: 148); 6) PATQ (SEQ ID NO: 149); 7) GKS; 8) YT; 9) CHP; 10) HRPA (SEQ ID NO: 150); 11) LLGSE (SEQ ID NO: 151); 12) GLRDASGV (SEQ ID NO: 152); 13) SSGKSAVQGP (SEQ ID NO: 153); 14) GCYS (SEQ ID NO: 154); 15) CAEP (SEQ ID NO: 155); 16) PE; 17) SGNTFRPEVHLLPPPSEELALNEL (SEQ ID NO: 156); 18) ARGFS (SEQ ID NO:157); 19) QGSQELPREKY (SEQ ID NO:158); 20) AV; 21) AAED (SEQ ID NO: 159); 22) HEAL (SEQ ID NO: 160); and 23) IDRLAGKPTHVNVSVVMAEVDGTCY (SEQ ID NO: 161).

A sulfatase motif can be provided within or adjacent one or more of these amino acid sequences of such modification sites of an Ig heavy chain. For example, an Ig heavy chain polypeptide amino acid sequence can be modified (e.g., where the modification includes one or more amino acid residue insertions, deletions, and/or substitutions) at one or more of these amino acid sequences to provide a sulfatase motif adjacent and N-terminal and/or adjacent and C-terminal to these modification sites. Alternatively or in addition, an Ig heavy chain polypeptide amino acid sequence can be modified (e.g., where the modification includes one or more amino acid residue insertions, deletions, and/or substitutions) at one or more of these amino acid sequences to provide a sulfatase motif between any two residues of the Ig heavy chain modifications sites. In some embodiments, an Ig heavy chain polypeptide amino acid sequence may be modified to include two motifs, which may be adjacent to one another, or which may be separated by one, two, three, four or more (e.g., from about 1 to about 25, from about 25 to about 50, or from about 50 to about 100, or more, amino acids. Alternatively or in addition, where a native amino acid sequence provides for one or more amino acid residues of a sulfatase motif sequence, selected amino acid residues of the modification sites of an Ig heavy chain polypeptide amino acid sequence can be modified (e.g., where the modification includes one or more amino acid residue insertions, deletions, and/or substitutions) so as to provide a sulfatase motif at the modification site.

The amino acid sequence of a surface-accessible loop region can thus be modified to provide a sulfatase motif, where the modifications can include insertions, deletions, and/or substitutions. For example, where the modification is in a CH1 domain, the surface-accessible loop region can have the amino acid sequence NSGALTSG (SEQ ID NO: 71), and the aldehyde-tagged sequence can be, e.g., NSGALCTPSRG (SEQ ID NO: 162), e.g., where the “TS” residues of the NSGALTSG (SEQ ID NO: 71) sequence are replaced with “CTPSR,” such that the sulfatase motif has the sequence LCTPSR. As another example, where the modification is in a CH2 domain, the surface-accessible loop region can have the amino acid sequence NKALPAP (SEQ ID NO: 84), and the aldehyde-tagged sequence can be, e.g., NLCTPSRAP (SEQ ID NO: 163), e.g., where the “KAL” residues of the NKALPAP sequence are replaced with “LCTPSR,” such that the sulfatase motif has the sequence LCTPSR. As another example, where the modification is in a CH2/CH3 domain, the surface-accessible loop region can have the amino acid sequence KAKGQPR (SEQ ID NO: 86), and the aldehyde-tagged sequence can be, e.g., KAKGLCTPSR (SEQ ID NO: 192), e.g., where the “GQP” residues of the KAKGQPR sequence are replaced with “LCTPS,” such that the sulfatase motif has the sequence LCTPSR.

As noted above, an isolated aldehyde-tagged antibody, such as anti-CD30 antibody can comprise a light chain constant region amino acid sequence modified to include a sulfatase motif as described above, where the sulfatase motif is in or adjacent a surface-accessible loop region of the Ig polypeptide light chain constant region.

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an Ig light chain constant region corresponding to one or more of: 1) amino acids 130-135; 2) amino acids 141-143; 3) amino acid 150; 4) amino acids 162-166; 5) amino acids 163-166; 6) amino acids 173-180; 7) amino acids 186-194; 8) amino acids 211-212; 9) amino acids 220-225; 10) amino acids 233-236; wherein the amino acid numbering is based on the amino acid numbering of human kappa light chain. In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of an Ig light chain constant region corresponding to one or more of: 1) amino acids 1-6; 2) amino acids 12-14; 3) amino acid 21; 4) amino acids 33-37; 5) amino acids 34-37; 6) amino acids 44-51; 7) amino acids 57-65; 8) amino acids 83-83; 9) amino acids 91-96; 10) amino acids 104-107; where the amino acid numbering is based on the human kappa light chain.

Exemplary surface-accessible loop regions of an Ig light chain (e.g., a human kappa light chain) include: 1) RTVAAP (SEQ ID NO: 165); 2) PPS; 3) Gly (see, e.g., Gly at position 150 of the human kappa light chain sequence); 4) YPREA (SEQ ID NO: 166); 5) PREA; 6) DNALQSGN (SEQ ID NO: 167); 7) TEQDSKDST (SEQ ID NO: 168); 8) HK; 9) HQGLSS (SEQ ID NO: 169); and 10) RGEC (SEQ ID NO: 170).

Exemplary surface-accessible loop regions of an Ig lambda light chain include QPKAAP (SEQ ID NO: 171), PPS, NK, DFYPGAV (SEQ ID NO: 172), DSSPVKAG (SEQ ID NO: 173), TTP, SN, HKS, EG, and APTECS (SEQ ID NO: 174).

In some instances, a target immunoglobulin amino acid sequence is modified to include a sulfatase motif as described above, where the modification includes one or more amino acid residue insertions, deletions, and/or substitutions. In certain embodiments, the sulfatase motif is within, or adjacent to, a region of a rat Ig light chain constant region corresponding to one or more of: 1) amino acids 1-6; 2) amino acids 12-14; 3) amino acids 121-22; 4) amino acids 31-37; 5) amino acids 44-51; 6) amino acids 55-57; 7) amino acids 61-62; 8) amino acids 81-83; 9) amino acids 91-92; 10) amino acids 102-105; wherein the amino acid numbering is based on the amino acid numbering of rat light chain.

In some cases, a sulfatase motif is introduced into the CH1 region of a heavy chain constant region. In some cases, a sulfatase motif is introduced at or near (e.g., within 1 to 10 amino acids of) the C-terminus of a heavy chain. In some cases, a sulfatase motif is introduced in the light-chain constant region.

In some cases, a sulfatase motif is introduced into the CH1 region of an anti-CD30 heavy chain constant region, e.g., within amino acids 121-219 of the IgG1 heavy chain amino acid sequence. For example, in some cases, a sulfatase motif is introduced into the amino acid sequence: ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVE (SEQ ID NO: 175). For example, in some of these embodiments, the amino acid sequence GALTSGVH (SEQ ID NO: 193) is modified to GALCTPSRGVH (SEQ ID NO: 194), where the sulfatase motif is LCTPSR (SEQ ID NO: 58).

In some cases, a sulfatase motif is added before the asparagine residue at the 91^{st} position in SEQ ID NO: 175. The sulfatase motif can be LCTPSR (SEQ ID NO: 58). For example, the sulfatase motif LCTPSR (SEQ ID NO: 58) can be added before the asparagine residue at the 91^{st} position in SEQ ID NO: 175 to produce an antibody comprising the sequence of KPSLCTPSRNTK (SEQ ID NO: 189). This insertion site for the sulfatase motif is referenced herein as 91N.

Certain details of insertion of a sulfatase motif at the 91N site are described in the U.S. Patent Application Publication No. 20200010561, which is incorporated herein by reference in its entirety.

For example, the sulfatase motif LCTPSR (SEQ ID NO: 58) can be added before the asparagine residue at the 91^{st} position in SEQ ID NO: 175 to produce an antibody comprising the following sequence (sulfatase motif is highlighted in bold and italics, 91^{st} asparagine residue is highlighted in bold).

(SEQ ID NO: 190)
ASTKGPSVFPLAPSSKSTSGGTAALGCLVKDYFPEPVTVSWNSGALTSG
VHTFPAVLQSSGLYSLSSVVTVPSSSLGTQTYICNVNHKPS [symbol] N
TKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSF
FLYSKLTVDKSRWQQGNVFSCSVMHEALHNHYTQKSLSLSPGK.

In some cases, a sulfatase motif is added before the 91^{st} asparagine residue in a sequence having at least 90%, 91%, 92%, 93%, 94%, 95%, 96%, 97%, 98%, or 99% sequence identity to SEQ ID NO: 175. Based on the sequence alignment of such sequence with the sequence of SEQ ID NO: 175, a person of ordinary skill in the art can readily determine the position of the 91^{st} asparagine residue and, thus, determine the insertion site for the sulfatase motif.

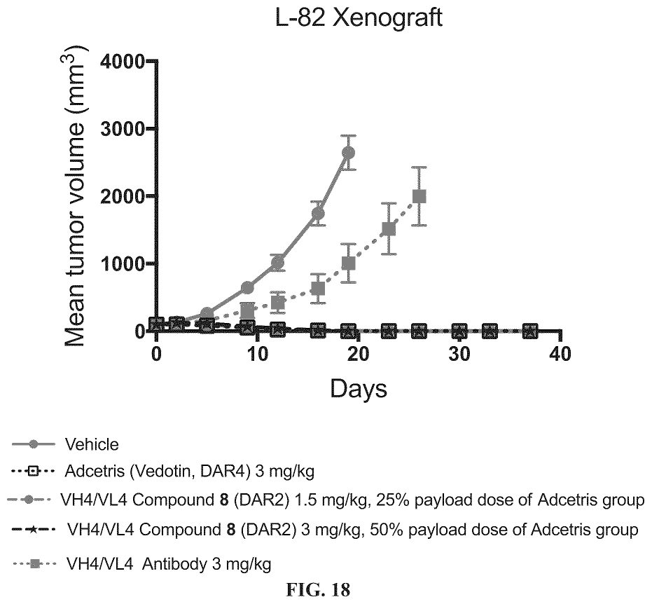

Non-limiting examples of antibodies having an insertion of a sulfatase motif at the 91N site is provided in Examples 9-10 described below and FIGS. 18-21.

In some cases, a sulfatase motif is introduced at or near the C-terminus of an antibody heavy chain, e.g., the sulfatase motifs introduced within 1 amino acid, 2 amino acids (aa), 3 aa, 4 aa, 5 aa, 6 aa, 7 aa, 8 aa, 9 aa, or 10 aa the C-terminus of an antibody heavy chain. As one non-limiting example, the C-terminal lysine reside of an antibody heavy chain can be replaced with the amino acid sequence SLCTPSRGS (SEQ ID NO: 195).

In some cases, a sulfatase motif is introduced into the constant region of a light chain of an anti-CD30 antibody. As one non-limiting example, in some cases, a sulfatase motif is introduced into the constant region of a light chain of an anti-CD30 antibody, where the sulfatase motif is C-terminal to KVDNAL (SEQ ID NO: 62), and/or is N-terminal to QSGNSQ (SEQ ID NO: 63). For example, in some cases, the sulfatase motif is LCTPSR (SEQ ID NO: 58), and the anti-CD30 light chain comprises the amino acid sequence KVDNALLCTPSRQSGNSQ (SEQ ID NO: 176).

### Drugs

In some cases, a binding agent of the present disclosure comprises drug covalently linked to the heavy and/or light chain of the antibody. “Drugs” include small molecule drugs, peptidic drugs, toxins (e.g., cytotoxins), and the like. In some cases, the binding agent of the present disclosure has a drug (e.g., W¹ in conjugates of formula (I) and formula (Ia) described herein, or W¹¹ or W¹² in conjugates of formula (II) described herein) covalently linked to the heavy and/or light chain of the antibody. For example, a binding agent (e.g., antibody) conjugate of the present disclosure can include as substituent W¹, W¹¹ or W¹² a drug or active agent as described herein.

“Small molecule drug” as used herein refers to a compound, e.g., an organic compound, which exhibits a pharmaceutical activity of interest and which is generally of a molecular weight of no greater than about 800 Da, or no greater than 2000 Da, but can encompass molecules of up to 5 kDa and can be as large as about 10 kDa. A small inorganic molecule refers to a molecule containing no carbon atoms, while a small organic molecule refers to a compound containing at least one carbon atom.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight.

In some cases, the drug is a toxin, e.g., a cytotoxin. Ribosome inactivating proteins (RIPs), which are a class of proteins ubiquitous in higher plants, are examples of such cytotoxins. Suitable cytotoxins include, but are not limited to, ricin, abrin, diphtheria toxin, a Pseudomonas exotoxin (e.g., PE35, PE37, PE38, PE40, etc.), saporin, gelonin, a pokeweed anti-viral protein (PAP), botulinum toxin, bryodin, momordin, and bouganin.

In some cases, the drug is a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones. Peptidic compounds can also be used.

Suitable cancer chemotherapeutic agents include dolastatin and active analogs and derivatives thereof; and auristatin and active analogs and derivatives thereof. Suitable cancer chemotherapeutic agents also include maytansinoids and active analogs and derivatives thereof; and duocarmycins and active analogs and derivatives thereof.

In certain embodiments, the drug or active agent can be a maytansine. “Maytansine”, “maytansine moiety”, “maytansine active agent moiety” and “maytansinoid” refer to a maytansine and analogs and derivatives thereof, and pharmaceutically active maytansine moieties and/or portions thereof. A maytansine conjugated to the polypeptide can be any of a variety of maytansinoid moieties such as, but not limited to, maytansine and analogs and derivatives thereof as described herein (e.g., deacylmaytansine).

In certain embodiments, the drug or active agent can be an auristatin, or an analog or derivative thereof, or a pharmaceutically active auristatin moiety and/or a portion thereof. An auristatin conjugated to the polypeptide can be any of a variety of auristatin moieties such as, but not limited to, an auristatin and analogs and derivatives thereof as described herein. Examples of drugs that find use in the conjugates and compounds described herein include, but are not limited to an auristatin or an auristatin derivative, such as monomethyl auristatin D (MMAD), monomethyl auristatin E (MMAE), monomethyl auristatin F (MMAF), derivatives thereof, and the like.

In some cases, suitable cancer chemotherapeutic agents include topoisomerase inhibitors, such as, but not limited to camptothecine and derivatives thereof (e.g., topotecan, irinotecan, belotecan, exatecan, SN-38, silatecan, cositecan, lurtotecan, gimatecan, rubitecan, 9-aminocamptothecin (9-AC), and the like).

In certain embodiments, the drug W¹ in formula (I) and formula (Ia) described herein or W¹ or W¹² in formula (II) described herein is a camptothecine, or analog or derivative thereof. For example, in some instances, the camptothecine, or analog or derivative thereof, is a compound of formula (IV):

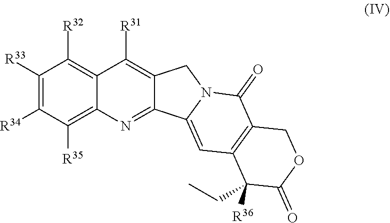

wherein:
R³¹ and R³² are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³¹ and R³² are optionally cyclically linked to form a 5 or 6-membered cycloalkyl or heterocyclyl ring;
R³³ and R³⁴ are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³³ and R³⁴ are optionally cyclically linked to form a 5 or 6-membered cycloalkyl or heterocyclyl ring;
R³⁵ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl;
R³⁶ is selected from OH and OC(O)R³⁷; and
R³⁷ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl.

In certain embodiments of formula (IV), the linker L in formula (I) or formula (Ia) described herein, or the first linker L^{A} or the second linker L^{B} in formula (II) described herein, is attached to a compound of formula (IV) at R³¹, R³², R³³, R³⁴, R³⁵ or R³⁶.

In certain embodiments, R³¹ and R³² are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³¹ and R³² are optionally cyclically linked to form a 5 or 6-membered cycloalkyl or heterocyclyl ring.

In certain embodiments, R³¹ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³¹ is hydrogen. In certain embodiments, R³¹ is halogen (e.g., F, Cl, Br, I). In certain embodiments, R³¹ is hydroxy. In certain embodiments, R³¹ is amino or substituted amino. In certain embodiments, R³¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³¹ is methyl. In certain embodiments, R³¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³¹ is alkynyl or substituted alkynyl. In certain embodiments, R³¹ is alkoxy or substituted alkoxy. In certain embodiments, R³¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³¹ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³² is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³² is hydrogen. In certain embodiments, R³² is halogen (e.g., F, Cl, Br, I). In certain embodiments, R³² is hydroxy. In certain embodiments, R³² is amino or substituted amino. In certain embodiments, R³² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³² is methyl. In certain embodiments, R³² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³² is alkynyl or substituted alkynyl. In certain embodiments, R³² is alkoxy or substituted alkoxy. In certain embodiments, R³² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³² is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³¹ and R³² are optionally cyclically linked to form a 5 or 6-membered cycloalkyl or heterocyclyl ring. In certain embodiments, R³¹ and R³² are cyclically linked to form a 5 or 6-membered cycloalkyl. In certain embodiments, R³¹ and R³² are cyclically linked to form a 5 or 6-membered heterocyclyl. In certain embodiments, R³¹ and R³² are cyclically linked to form a 5-membered cycloalkyl. In certain embodiments, R^{3′} and R³² are cyclically linked to form a 6-membered cycloalkyl. In certain embodiments, R^{3′} and R³² are cyclically linked to form a 5-membered heterocyclyl. In certain embodiments, R³¹ and R³² are cyclically linked to form a 6-membered heterocyclyl.

In certain embodiments, R³³ and R³⁴ are each independently selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl, or R³³ and R³⁴ are optionally cyclically linked to form a 5 or 6-membered cycloalkyl or heterocyclyl ring.

In certain embodiments, R³³ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³³ is hydrogen. In certain embodiments, R³³ is halogen (e.g., F, Cl, Br, I). In certain embodiments, R³³ is hydroxy. In certain embodiments, R³³ is amino or substituted amino. In certain embodiments, R³³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³³ is methyl. In certain embodiments, R³³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³³ is alkynyl or substituted alkynyl. In certain embodiments, R³³ is alkoxy or substituted alkoxy. In certain embodiments, R³³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³³ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³⁴ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³⁴ is hydrogen. In certain embodiments, R³⁴ is halogen (e.g., F, Cl, Br, I). In certain embodiments, R³⁴ is hydroxy. In certain embodiments, R³⁴ is amino or substituted amino. In certain embodiments, R³⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁴ is methyl. In certain embodiments, R³⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁴ is alkynyl or substituted alkynyl. In certain embodiments, R³⁴ is alkoxy or substituted alkoxy. In certain embodiments, R³⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁴ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³³ and R³⁴ are optionally cyclically linked to form a 5 or 6-membered cycloalkyl or heterocyclyl ring. In certain embodiments, R³³ and R³⁴ are cyclically linked to form a 5 or 6-membered cycloalkyl. In certain embodiments, R³³ and R³⁴ are cyclically linked to form a 5 or 6-membered heterocyclyl. In certain embodiments, R³³ and R³⁴ are cyclically linked to form a 5-membered cycloalkyl. In certain embodiments, R³³ and R³⁴ are cyclically linked to form a 6-membered cycloalkyl. In certain embodiments, R³³ and R³⁴ are cyclically linked to form a 5-membered heterocyclyl. In certain embodiments, R³³ and R³⁴ are cyclically linked to form a 6-membered heterocyclyl.

In certain embodiments, R³⁵ is selected from hydrogen, halogen, hydroxy, amino, substituted amino, alkyl, substituted alkyl, alkenyl, substituted alkenyl, alkynyl, substituted alkynyl, alkoxy, substituted alkoxy, aryl, substituted aryl, heteroaryl, substituted heteroaryl, cycloalkyl, substituted cycloalkyl, heterocyclyl, and substituted heterocyclyl. In certain embodiments, R³⁵ is hydrogen. In certain embodiments, R³⁵ is halogen (e.g., F, Cl, Br, I). In certain embodiments, R³⁵ is hydroxy. In certain embodiments, R³⁵ is amino or substituted amino. In certain embodiments, R³⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁵ is methyl. In certain embodiments, R³⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁵ is alkynyl or substituted alkynyl. In certain embodiments, R³⁵ is alkoxy or substituted alkoxy. In certain embodiments, R³⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁵ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R³⁶ is selected from OH and OC(O)R³⁷. In certain embodiments, R³⁶ is OH. In certain embodiments, R³⁶ is OC(O)R³⁷.

In certain embodiments, R³⁷ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R³⁷ is hydrogen. In certain embodiments, R³⁷ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³⁷ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³⁷ is alkynyl or substituted alkynyl. In certain embodiments, R³⁷ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³⁷ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³⁷ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R³⁷ is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, the compound of formula (IV) has the structure of formula (IVa):

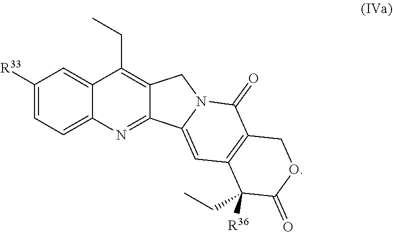

In certain embodiments of the compound of formula (IVa), R³³ is as described above.

In certain embodiments of the compound of formula (IVa), R³⁶ is as described above.

In certain embodiments of the compound of formula (IVa), R³³ is OH and L is attached at R³⁶. In certain embodiments of the compound of formula (IVa), L is attached at R³³ and R³⁶ is OH.

In certain embodiments, the compound of formula (IV) has the structure of formula (IVb):

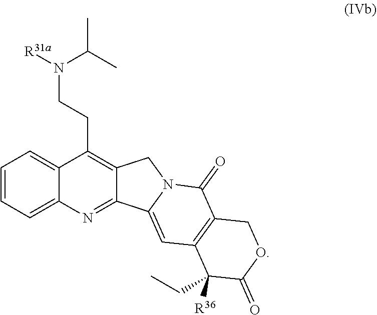

In certain embodiments of the compound of formula (IVb), R^{31a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{31a} is hydrogen. In certain embodiments, R^{31a} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{31a} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{31a} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{31a} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{31a} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{31a} is carboxyl. In certain embodiments, R^{31a} is carboxyl ester. In certain embodiments, R^{31a} is acyl. In certain embodiments, R^{31a} is sulfonyl.

In certain embodiments of the compound of formula (IVb), R³⁶ is as described above.

In certain embodiments of the compound of formula (IVb), R^{31a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and L is attached at R³⁶. In certain embodiments of the compound of formula (IVb), L is attached at R^{31a} and R³⁶ is OH.

In certain embodiments, the compound of formula (IV) has the structure of formula (IVc):

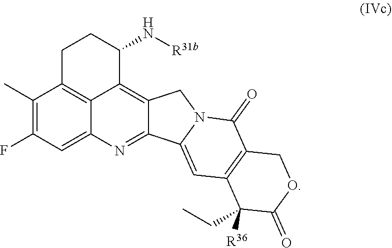

In certain embodiments of the compound of formula (IVc), R^{31b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{31b} is hydrogen. In certain embodiments, R^{31b} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{31b} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{31b} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{31b} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{31b} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{31b} is carboxyl. In certain embodiments, R^{31b} is carboxyl ester. In certain embodiments, R^{31b} is acyl. In certain embodiments, R^{31b} is sulfonyl.

In certain embodiments of the compound of formula (IVc), R³⁶ is as described above.

In certain embodiments of the compound of formula (IVc), R^{31b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and L is attached at R³⁶. In certain embodiments of the compound of formula (IVc), L is attached at R^{31b} and R³⁶ is OH.

In certain embodiments, the compound of formula (IV) has the structure of formula (IVd):

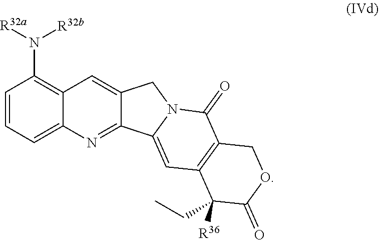

In certain embodiments of the compound of formula (IVd), R^{32a} and R^{32b} are each independently selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl.

In certain embodiments of the compound of formula (IVd), R^{32a} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{32a} is hydrogen. In certain embodiments, R^{32a} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{32a} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{32a} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{32a} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{32a} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{32a} is carboxyl. In certain embodiments, R^{32a} is carboxyl ester. In certain embodiments, R^{32a} is acyl. In certain embodiments, R^{32a} is sulfonyl.

In certain embodiments of the compound of formula (IVd), R^{32b} is selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl. In certain embodiments, R^{32b} is hydrogen. In certain embodiments, R^{32b} is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R^{32b} is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R^{32b} is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R^{32b} is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R^{32b} is heterocyclyl or substituted heterocyclyl, such as a C₃₋₆ heterocyclyl or C₃₋₆ substituted heterocyclyl, or a C₃₋₅ heterocyclyl or C₃₋₅ substituted heterocyclyl. In certain embodiments, R^{32b} is carboxyl. In certain embodiments, R^{32b} is carboxyl ester. In certain embodiments, R^{32b} is acyl. In certain embodiments, R^{32b} is sulfonyl.

In certain embodiments of the compound of formula (IVd), R³⁶ is as described above.

In certain embodiments of the compound of formula (IVd), R^{32a} and R^{32b} are each independently selected from H, alkyl, substituted alkyl, aryl, substituted aryl, heteroaryl, substituted heteroaryl, cycloalkyl, substituted cycloalkyl, heterocyclyl, substituted heterocyclyl, carboxyl, carboxyl ester, acyl, and sulfonyl, and L is attached at R³⁶. In certain embodiments of the compound of formula (IVd), L is attached at R^{32a} or R^{32b} and R³⁶ is OH. In certain embodiments of the compound of formula (IVd), L is attached at R^{32a} and R³⁶ is OH. In certain embodiments of the compound of formula (IVd), L is attached at R^{32b} and R³⁶ is OH.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and progestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity.

Other suitable chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., TAXOTERE™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives; piperazino and piperazino derivatives.

### Methods of Producing Antibody

A subject binding agent can be produced by any known method, e.g., conventional synthetic methods for protein synthesis; recombinant DNA methods, etc.

Where a subject binding agent is a single chain polypeptide, it can be synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody.

Standard recombinant methods can be used for production of a subject binding agent. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the code, a variety of nucleic acid sequences can encode each immunoglobulin amino acid sequence. The desired nucleic acid sequences can be produced by de novo solid-phase DNA synthesis or by polymerase chain reaction (PCR) mutagenesis of an earlier prepared variant of the desired polynucleotide.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species. Other microbes, such as yeast, are also useful for expression. Saccharomyces (e.g., S. cerevisiae) and Pichia are examples of suitable yeast host cells.

In addition to microorganisms, mammalian cells (e.g., mammalian cells grown in in vitro cell culture) can also be used to express and produce the polypeptides of the present invention (e.g., polynucleotides encoding immunoglobulins or fragments thereof). Suitable mammalian host cells include CHO cell lines, various Cos cell lines, HeLa cells, myeloma cell lines, and transformed B-cells or hybridomas. Expression vectors for these cells can include expression control sequences, such as an origin of replication, a promoter, and an enhancer, and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like.

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

### Compositions

The present disclosure provides a composition comprising a subject binding agent. A subject binding agent composition can comprise, in addition to a binding agent, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

### Nucleic Acids

The present disclosure provides nucleic acids comprising nucleotide sequences encoding a subject binding agent. A nucleotide sequence encoding a subject binding agent can be operably linked to one or more regulatory elements, such as a promoter and enhancer, that allow expression of the nucleotide sequence in the intended target cells (e.g., a cell that is genetically modified to synthesize the encoded antibody).

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHO5 promoter, a CUP1 promoter, a GAL7 promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like. Suitable strong promoters for use in prokaryotes such as Escherichia coli include, but are not limited to Trc, Tac, T5, T7, and P_{Lambda}. Non-limiting examples of operators for use in bacterial host cells include a lactose promoter operator (LacI repressor protein changes conformation when contacted with lactose, thereby preventing the LacI repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator.

A nucleotide sequence encoding a subject binding agent can be present in an expression vector and/or a cloning vector. Where a subject binding agent comprises two separate polypeptides, nucleotide sequences encoding the two polypeptides can be cloned in the same or separate vectors. An expression vector can include a selectable marker, an origin of replication, and other features that provide for replication and/or maintenance of the vector.

Large numbers of suitable vectors and promoters are known to those of skill in the art; many are commercially available for generating a subject recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

### Cells

The present disclosure provides isolated genetically modified host cells (e.g., in vitro cells) that are genetically modified with a subject nucleic acid. In some embodiments, a subject isolated genetically modified host cell can produce a subject binding agent.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), Vero cells, NIH 3T³ cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) 293 cells (ATCC No. CRL1573), HLHepG2 cells, and the like.

Suitable yeast cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like.

Suitable prokaryotic cells include, but are not limited to, any of a variety of laboratory strains of Escherichia coli, Lactobacillus sp., Salmonella sp., Shigella sp., and the like.

### Pharmaceutical Compositions

The present disclosure provides compositions, including pharmaceutical compositions, comprising a subject binding agent, such as an antibody comprising VH and VL sequences as described herein. In a specific embodiment, the pharmaceutical compositions comprise an anti-CD30 antibody comprising VH and VL framework regions described herein. In certain embodiments, the pharmaceutical compositions comprise an anti-CD30 antibody comprising at least one VH or VL chain having the sequences of SEQ ID NOs: 6, 11, 21, or 26.

In general, a formulation comprises an effective amount of a subject binding agent. An “effective amount” means a dosage sufficient to produce a desired result, e.g., reduction in the number of cancerous cells. In some cases, the desired result is at least a reduction in a symptom of a malignancy, as compared to a control.

### Formulations

In the subject methods, a subject binding agent can be administered to the host using any convenient means capable of resulting in the desired therapeutic effect or diagnostic effect. A binding agent can be an antibody comprising VH and VL sequences as described herein. In a specific embodiment, the binding agent is an anti-CD30 antibody comprising VH and VL framework regions described herein. In certain embodiments, the pharmaceutical compositions comprise an anti-CD30 antibody comprising at least one VH or VL chain having the sequences of SEQ ID NOs: 6, 11, 21, or 26.

The binding agent can be incorporated into a variety of formulations for therapeutic administration. More particularly, a binding agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a subject binding agent can be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, a subject binding agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A subject binding agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a subject binding agent are prepared by mixing the binding agent having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

Exemplary antibody concentrations in a subject pharmaceutical composition may range from about 1 mg/mL to about 200 mg/ml or from about 50 mg/mL to about 200 mg/mL, or from about 150 mg/mL to about 200 mg/mL.

An aqueous formulation of the binding agent may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes a subject binding agent, and one or more agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a subject formulation can be a liquid or lyophilized formulation suitable for parenteral administration, and can comprise: about 1 mg/mL to about 200 mg/mL of a subject antibody; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a subject parenteral formulation is a liquid or lyophilized formulation comprising: about 1 mg/mL to about 200 mg/mL of a subject antibody; 0.04% Tween 20 w/v; 20 mM L-histidine; and 250 mM Sucrose; and has a pH of 5.5.

The term “unit dosage form,” as used herein, refers to physically discrete units suitable as unitary dosages for human and animal subjects, each unit containing a predetermined quantity of binding agents of the present invention calculated in an amount sufficient to produce the desired effect in association with a pharmaceutically acceptable diluent, carrier or vehicle. The specifications for a subject binding agent may depend on the particular antibody employed and the effect to be achieved, and the pharmacodynamics associated with each binding agent in the host.

A subject binding agent can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the antibody encapsulated in liposome vehicles.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a subject binding agent is formulated in a controlled release formulation. Sustained-release preparations may be prepared using methods well known in the art. Suitable examples of sustained-release preparations include semipermeable matrices of solid hydrophobic polymers containing the antibody in which the matrices are in the form of shaped articles, e.g. films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous).

### Dosages

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. A subject binding agent may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute.

Those of skill will readily appreciate that dose levels can vary as a function of the specific binding agent, the severity of the symptoms and the susceptibility of the subject to side effects. Preferred dosages for a given compound are readily determinable by those of skill in the art by a variety of means.

### Routes of Administration

A subject binding agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the antibody and/or the desired effect. A subject antibody composition can be administered in a single dose or in multiple doses. In some embodiments, a subject antibody composition is administered orally. In some embodiments, a subject antibody composition is administered via an inhalational route. In some embodiments, a subject antibody composition is administered intranasally. In some embodiments, a subject antibody composition is administered locally. In some embodiments, a subject antibody composition is administered intracranially. In some embodiments, a subject antibody composition is administered intravenously.

The binding agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrahepatic, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

A subject binding agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as a breast cancer, pancreatic cancer, or lung cancer. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

In some embodiments, a subject binding agent is administered by injection, e.g., for systemic delivery (e.g., intravenous infusion) or to a local site.

A variety of hosts (wherein the term “host” is used interchangeably herein with the terms “subject,” “individual,” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the hosts will be humans.

### Treatment Methods

The present disclosure provides methods of treating a disease or disorder associated with or caused by cell a CD30-positive cell, e.g., a cancerous CD30-positive cell; an autoreactive CD30-positive cell.

Because the VH and VL chains comprising framework regions disclosed herein can be used to produce antibodies having specificity to any target, such antibodies could be used in methods of treating a disease or disorder that can be treated by administering antibodies that bind to a target of interest.

### Treating Malignancies

The present disclosure provides methods of treating a malignancy, including a solid tumor or a hematologic malignancy, the methods generally involving administering to an individual in need thereof (e.g., an individual having a malignancy) an effective amount of a subject binding agent, alone (e.g., in monotherapy) or in combination (e.g., in combination therapy) with one or more additional therapeutic agents.

Malignancies include, e.g., HCC, non-Hodgkin's lymphoma, Burkitt's lymphoma, multiple myeloma, chronic lymphocytic leukemia, hairy cell leukemia, prolymphocytic leukemia, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, vulvar cancer, and the like.

In some embodiments, an effective amount of a subject binding agent is an amount that, when administered alone (e.g., in monotherapy) or in combination (e.g., in combination therapy) with one or more additional therapeutic agents, in one or more doses, is effective to reduce the number of cancerous cells in an individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the number of cancerous cells in the individual in the absence of treatment with the binding agent.

### Combination Therapy

In some embodiments, a subject method of treating a malignancy involves administering a subject binding agent and one or more additional therapeutic agents. Suitable additional therapeutic agents include, but are not limited to, a cancer chemotherapeutic agent (as described above).

In some cases, the additional therapeutic agent is an immunomodulatory therapeutic agent, such as checkpoint inhibitor or an interleukin. An immune checkpoint inhibitor inhibits the function of an immune inhibitory checkpoint molecule, such as a protein. An immune checkpoint inhibitor can be an antibody that specifically binds to an immune checkpoint protein. Various immune checkpoint inhibitors are known. Immune checkpoint inhibitors include, but are not limited to, peptides, antibodies, nucleic acid molecules and small molecules.

Any suitable checkpoint inhibitor could be used in the methods disclosed herein. Examples of inhibitory checkpoint molecules include A2AR, B7-H3, Bf7-H4, BTLA, CTLA-4, CD277, IDO, KIR, PD-1, LAG-3, TIM-3, TIGIT and VISTA.

In some embodiments, an immune checkpoint inhibitor inhibits PD-1 signaling, for example, via inhibiting PD-1 or PD-L1. In some embodiments, an agent that inhibits PD-1 signaling is an anti-PD-1 antibody. In some embodiments, an anti-PD-1 antibody is nivolumab, pembrolizumab, atezolizumab, durvalumab, or avelumab. In some embodiments, an immune checkpoint inhibitor that inhibit PD-L1 includes, for example, AMP-244, MEDI-4736, MPDL328 OA, and MIH1.

In some embodiments, an immune checkpoint inhibitor is an inhibitor of CTLA-4, such as an antibody that targets CTLA-4, for example, ipilimumab.

In some embodiments, a checkpoint inhibitor targets CD366, which is a transmembrane protein also known as T cell immunoglobulin and mucin domain containing protein-3 (TIM-3).

Additional examples and certain aspects of immune checkpoint inhibitors are described by Hui (2019), Immune checkpoint inhibitors, J. Cell Biol., Vol. 218 No. 3 740-741, which is incorporated herein by reference in its entirety.

### Subjects Suitable for Treatment

A variety of subjects are suitable for treatment with a subject method. Suitable subjects include any individual, e.g., a human, who has a malignancy; who has been diagnosed with a malignancy; who has had a malignancy and is at risk for recurrence of the malignancy; who has been treated for a malignancy with an agent other than a subject binding agent (e.g., who has been treated with a cancer chemotherapeutic agent) and who has not responded to the agent; or who has been treated for a malignancy with an agent other than a subject binding agent (e.g., who has been treated with a cancer chemotherapeutic agent) and who initially responded to the agent but subsequently ceased to respond (e.g., relapsed).

### Detection Methods

The present disclosure provides various detection methods that involve use of a subject binding agent. Detection methods include diagnostic methods, prognostic methods, and monitoring methods. A subject detection method generally involves detecting cells that express a target of the subject binding agents, e.g., cancerous cells.

In some embodiments, a subject method is a diagnostic method, e.g., to determine whether an individual has a malignancy.

In some embodiments, a subject method is a monitoring method, e.g., an individual who has been diagnosed as having a malignancy, and is being treated for the disorder, is monitored for response to the treatment and/or progression/regression of the disorder.

In some cases, a subject detection method involves administering to an individual a detectably labeled binding agent of the present disclosure, particularly, anti-CD30 antibody; and detecting binding of the binding agent to tissues in the individual. Detection can be achieved, e.g., by magnetic resonance imaging or other suitable imaging technique.

In other instances, a subject detection method involves contacting a detectably labeled binding agent, for example, CD30 antibody, of the present disclosure with a biological sample obtained from an individual; and detecting binding of the binding agent to molecules in the biological sample.

The binding agent, for example, CD30 antibody, can be labeled directly or indirectly. Indirect labels include a secondary antibody that comprises a detectable label, where the secondary antibody binds a subject binding agent. Other indirect labels include biotin, where a biotinylated binding agent, such as anti-CD30 antibody, can be detected using avidin or streptavidin that comprises a detectable label.

Suitable detectable labels include any composition detectable by spectroscopic, photochemical, biochemical, immunochemical, electrical, optical or chemical means. Suitable include, but are not limited to, magnetic beads (e.g. Dynabeads™), fluorescent dyes (e.g., fluorescein isothiocyanate, texas red, rhodamine, a green fluorescent protein, a red fluorescent protein, a yellow fluorescent protein, and the like), radiolabels (e.g., ³H, ¹²⁵, ³⁵S, ¹⁴C, or ²P), enzymes (e.g., horseradish peroxidase, alkaline phosphatase, luciferase, and others commonly used in an enzyme-linked immunosorbent assay (ELISA)), and colorimetric labels such as colloidal gold or colored glass or plastic (e.g. polystyrene, polypropylene, latex, etc.) beads.

### EMBODIMENTS

Certain embodiments of the present disclosure are described in the clauses listed below. These embodiments are illustrative only and not intended to be limiting in scope.

1. A binding agent that specifically binds to CD30 protein, the binding agent comprising:

i) a variable heavy chain (VH) chain comprising a
sequence selected from:
(SEQ ID NO: 6)
QVQLQQSGPEVVKPGASVKVSCKASGYTFTDYYMTWVRQKPGQGLEW
MGWIYPGSGNTKYNQKFKGRVTITVDTSSSTAFMELSSLTSEDTAVYFC
ANYGNYWFAYWGQGTQVTVSA,
and
(SEQ ID NO: 11)
QVQLVQSGAEVKKPGASVKVSCKASGYTFTDYYITWVRQAPGQGLEWM
GWIYPGSGNTKYNEKFKGRVTITVDTSASTAYMELSSLRSEDTAVYYCA
NYGNYWFAYWGQGTLVTVSS;
and
ii) a variable light chain (VL) chain comprising
a sequence selected from:
(SEQ ID NO: 21)
DIVMTQSPASLAVSLGERATISCKSSQSVDFDGDSYLNWYQQKPGQPPK
LLIYAASTRESGVPARFSGSGSGTDFTLTISSLQEEDVATYYCQQSNED
PWTFGGGTKVEIK,
and
(SEQ ID NO: 26)
DIVLTQSPDSLAVSLGERATINCKASQSVDFDGDSYMNWYQQKPGQPPK
LLIYAASNRESGVPDRFSGSGSGTDFTLTISSLQAEDVAVYYCQQSNED
PWTFGGGTKVEIK.

2. The binding agent of clause 1, comprising a VH chain comprising SEQ ID NO: 11 and VL chain comprising SEQ ID NO: 26.

3. A binding agent that specifically binds to CD30, comprising:
VH chain comprising H-CDR1, H-CDR2, and H-CDR3 having the sequences of SEQ ID NOs: 31-33, respectively; and VL chain comprising L-CDR1, L-CDR2, and L-CDR3 having the sequences of SEQ ID NOs: 34-36, respectively; and wherein,
the VH chain comprises:
i) a heavy chain framework region 1 (HFR1) having the sequence of SEQ ID NO: 7, a heavy chain framework region 2 (HFR2) having the sequence of SEQ ID NO: 8, a heavy chain framework region 3 (HFR3) having the sequence of SEQ ID NO: 9, and a heavy chain framework region 4 (HFR4) having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and
the VL chain comprises:
i) a light chain framework region 1 (LFR1) having the sequence of SEQ ID NO: 22, a light chain framework region 2 (LFR2) having the sequence of SEQ ID NO: 23, a light chain framework region 3 (LFR3) having the sequence of SEQ ID NO: 24, and a light chain framework region 4 (LFR4) having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 25.

4. A binding agent that specifically binds to an antigen, comprising:
a VH chain comprising H-CDR1, H-CDR2, and H-CDR3 and a VL chain comprising L-CDR1, L-CDR2, and L-CDR3, wherein the complementarity determining regions determining the binding specificity of the binding agent for the antigen, and wherein, in the binding agent:
the VH chain comprises:
i) a HFR1 having the sequence of SEQ ID NO: 7, a HFR2 having the sequence of SEQ ID NO: 8, a HFR3 having the sequence of SEQ ID NO: 9, and a HFR4 having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and
the VL chain comprises:
i) a LFR1 having the sequence of SEQ ID NO: 22, a LFR2 having the sequence of SEQ ID NO: 23, a LFR3 having the sequence of SEQ ID NO: 24, and a LFR4 having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 30.

5. The binding agent of clause 1, wherein the binding agent specifically binds to CD30, and comprises: a VH chain comprising H-CDR1, H-CDR2, and H-CDR3 having the sequences of SEQ ID NOs: 31-33, respectively; and VL chain comprising L-CDR1, L-CDR2, and L-CDR3 having the sequences of SEQ ID NOs: 34-36, respectively.

6. The binding agent of any one of clauses 1-5, wherein the binding agent is a chimeric antibody.

7. The binding agent of any one of clauses 1-6, wherein the binding agent is selected from the group consisting of: T-cell receptor, T-cell receptor like antibody, an IgG, Fv, single chain antibody, scFv, Fab, F(ab′)2, or Fab′.

8. The binding agent of any one of clauses 1-7, wherein the binding agent is an IgG.

9. The binding agent of clause 8, wherein the IgG is an IgG1.

10. The binding agent of any one of clauses 1-7, wherein the binding agent is a Fab.

11. The binding agent of any one of clauses 1-7, wherein the binding agent is an scFv.

12. A bispecific binding agent comprising a first antigen-binding domain that specifically binds CD30, and wherein the first antigen binding domain comprises a VH chain and a VL chain as defined in any one of clauses 1 to 5.

13. The binding agent of any one of clauses 1-12, wherein the binding agent is detectably labeled.

14. The binding agent of any one of clauses 1-13, wherein the binding agent comprises a covalently linked non-peptide synthetic polymer.

15. The binding agent of clause 14, wherein the synthetic polymer is poly(ethylene glycol) polymer.

16. The binding agent of any one of clauses 1-15, wherein the antibody comprises a covalently linked lipid or fatty acid moiety.

17. The binding agent of any one of clauses 1-16, wherein the binding agent comprises a covalently linked polysaccharide or carbohydrate moiety.

18. The binding agent of any one of clauses 1-17, wherein the binding agent comprises a contrast agent.

19. The binding agent of any one of clauses 1-18, wherein the binding agent further comprises an affinity domain.

20. The binding agent of any one of clauses 1-19, wherein the binding agent is immobilized on a solid support.

21. The binding agent of any one of clauses 1-20, wherein the binding agent comprises a covalently linked cytotoxin.

22. The binding agent of any one of clauses 1-21, wherein the binding agent comprises a constant region amino acid sequence comprising an amino acid sequence of a sulfatase motif.

23. The binding agent of any one of clauses 1-22, wherein the binding agent is an antibody comprising a constant region amino acid sequence comprising an amino acid sequence of a sulfatase motif, and wherein the sulfatase motif is modified to comprise a 2-formylglycine (fGly) moiety.

24. The binding agent of clause 23, comprising the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the antibody, X¹ is present; and
X² and X³ are each independently any amino acid.

25. The binding agent of clause 24, wherein the sequence is L(fGly)TPSR.

26. The binding agent antibody of clause 24, wherein
Z³⁰ is selected from R, K, H, A, G, L, V, I, and P;
X¹ is selected from L, M, S, and V; and
X² and X³ are each independently selected from S, T, A, V, G, and C.

27. The binding agent of any one of clauses 24 to 26, wherein the sequence is at a C-terminus of a heavy chain constant region of the antibody.

28. The binding agent of clause 27, wherein the heavy chain constant region comprises the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid,
wherein the sequence is C-terminal to the amino acid sequence SLSLSPG.

29. The binding agent of clause 27, wherein the heavy chain constant region comprises the sequence SPGSL(fGly)TPSRGS.

30. The binding agent of clause 27, wherein
Z³⁰ is selected from R, K, H, A, G, L, V, I, and P;
X¹ is selected from L, M, S, and V; and
X² and X³ are each independently selected from S, T, A, V, G, and C.

31. The binding agent of any one of clauses 24 to 26, wherein the fGly moiety is positioned in a light chain constant region of the antibody.

32. The binding agent of clause 31, wherein the light chain constant region comprises the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the sequence KVDNAL, and/or is N-terminal to the sequence QSGNSQ.

33. The binding agent of clause 32, wherein the light chain constant region comprises the sequence KVDNAL(fGly)TPSRQSGNSQ.

34. The binding agent of clause 33, wherein
Z³⁰ is selected from R, K, H, A, G, L, V, I, and P;
X¹ is selected from L, M, S, and V; and
X² and X³ are each independently selected from S, T, A, V, G, and C.

35. The binding agent of any one of clauses 24 to 26, wherein the fGly moiety is positioned in a heavy chain CH1 region of the antibody.

36. The binding agent of clause 35, wherein the heavy chain CH1 region comprises the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the amino acid sequence SWNSGA and/or is N-terminal to the amino acid sequence GVHTFP.

37. The binding agent of clause 36, wherein the heavy chain CH1 region comprises the sequence SWNSGAL(fGly)TPSRGVHTFP.

38. The binding agent of clause 36, wherein
Z³⁰ is selected from R, K, H, A, G, L, V, I, and P;
X¹ is selected from L, M, S, and V; and
X² and X³ are each independently selected from S, T, A, V, G, and C.

39. The binding agent of any one of clauses 35-36 and 38, wherein the binding agent comprises the sequence X¹(fGly)X²Z²⁰X³Z³⁰ before the asparagine residue at the 91^{st} position of the heavy chain CH1 region.

40. The binding agent of any one of clauses 35-36 and 38-39, wherein the heavy chain CH1 region comprises the sequence of SEQ ID NO: 175 or a sequence having at least 90% sequence identity to the sequence of SEQ ID NO: 175.

41. The binding agent of clause 39 or 40, wherein the sequence X¹(fGly)X²Z²⁰X³Z³⁰ comprises the sequence LCTPSR (SEQ ID NO: 58).

42. The binding agent of clause 41, comprising the sequence LCTPSR (SEQ ID NO: 58) before the asparagine residue at the 91^{st} position of SEQ ID NO: 175 to produce an antibody comprising the sequence of KPSLCTPSRNTK (SEQ ID NO: 189).

43. The binding agent of clause 42, comprising the following sequence:

(SEQ ID NO: 190)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSLCTPSRNT
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

44. The binding agent of any one of clauses 24 to 26, wherein the fGly moiety is positioned in a heavy chain CH2 region of the antibody.

45. The binding agent of any one of clauses 24 to 26, wherein the fGly moiety is positioned in a heavy chain CH3 region of the antibody.

46. The binding agent of any one of clauses 23 to 45, wherein the binding agent comprises a heterologous moiety covalently linked to the antibody via the fGly moiety.

47. The binding agent of clause 46, wherein the heterologous moiety is a drug, oligonucleotide, protein, lipid nanoparticle, viral particle, a toxin, a detectable label, a water-soluble polymer, or a synthetic peptide.

48. A nucleic acid encoding a variable heavy (VH) chain, a variable light chain (VL), or both, of the binding agent of any one of clauses 1 to 47.

49. The nucleic acid of clause 48, wherein the binding agent is a single chain antibody, and wherein the nucleic acid encodes the single chain antibody.

50. The nucleic acid of clause 49, wherein the single chain antibody is an scFv.

51. A recombinant expression vector comprising the nucleic acid of any one of clauses 48-50, wherein the nucleic acid is operably linked to a transcriptional control element that is active in a eukaryotic cell.

52. A cell comprising the nucleic acid of any one of clauses 48 to 50 or the expression vector of Claim 51.

53. The cell of clause 52, wherein the nucleic acid encodes the VH chain and the VL chain of the binding agent.

54. The cell of clause 53, wherein the binding agent is a single chain antibody, and wherein the nucleic acid encodes the single chain antibody.

55. The cell of clause 54, wherein the single chain antibody is an scFv.

56. A cell comprising:
a first nucleic acid encoding a VH chain of a binding agent; and
a second nucleic acid encoding a VL chain of the binding agent,
wherein the VH chain and the VL chain produces the binding agent according to any of clauses 1 to 47.

57. The cell of clause 56, comprising:
a first expression vector comprising the first nucleic acid; and
a second expression vector comprising the second nucleic acid.

58. A fusion protein, comprising:
a VH chain, a VL chain, or both, of the binding agent of any one of clauses 1-47; fused to
a heterologous amino acid sequence.

59. A conjugate, comprising:
the binding agent of any one of clauses 1-47; and
an agent conjugated to the binding agent.

60. The conjugate of clause 59, wherein the agent is selected from the group consisting of: a half-life extending moiety, a labeling agent, and a drug.

61. The conjugate of clause 60, wherein the conjugate is of formula (I):

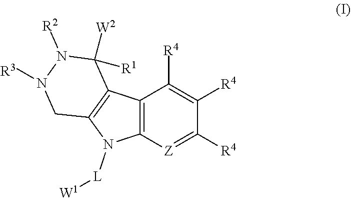

wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker;
W¹ is a drug; and
W² is the binding agent.

62. The conjugate of clause 61, wherein L comprises:
-(T¹-V¹)ₐ-(T²-V²)_{b}-(T³-V³)_{c}-(T⁴-V⁴)_{d}-(T⁵-V⁵)ₑ-(T⁷-V⁷)_{f}—,
   wherein
a, b, c, d, e and f are each independently 0 or 1, wherein the sum of a, b, c, d, e and f is 1 to 6;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹—, —NR⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

63. The conjugate of clause 62, wherein:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), MABO, MABC, PABO, PABC, PAB, PABA, PAP, PHP, an acetal group, a hydrazine, and an ester; and
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is

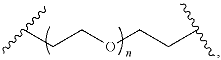

where n is an integer from 1 to 30;
EDA is an ethylene diamine moiety having the following structure:

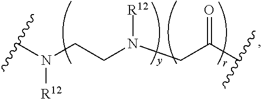

where y is an integer from to 6 and r is 0 or 1;
4-amino-piperidine (4AP) is

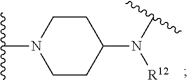

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

64. The conjugate of any of clauses 62 to 63, wherein MABO, MABC, PABO, PABC, PAB, PABA, PAP and PHP are each optionally substituted with a glycoside.

65. The conjugate of clause 58, wherein the glycoside is selected from a glucuronide, a galactoside, a glucoside, a mannoside, a fucoside, O-GlcNAc, and O-GalNAc.

66. The conjugate of any of clauses 62 to 65,

wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0.

67. The conjugate of any of clauses 61 to 66, wherein the drug is monomethyl auristatin E (MMAE).

68. The conjugate of any one of clauses 61 to 67, wherein the conjugate has the structure:

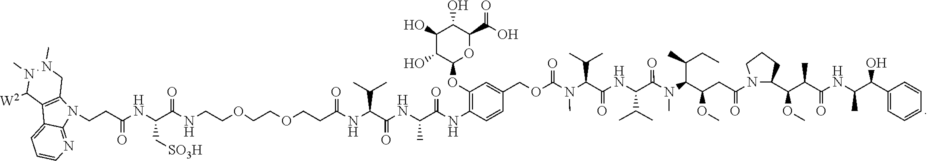

69. The conjugate of clause 60, wherein the conjugate is of formula (Ia):

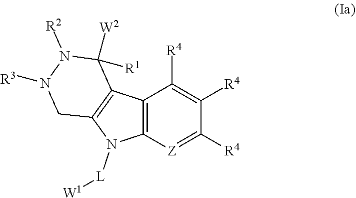

wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker comprising -(T¹-V¹)ₐ-(T²-V²)_{b}-(T³-V³)_{c}-(T⁴-V⁴)_{d}—, wherein a, b, c and d are each independently 0 or 1, where the sum of a, b, c and d is 1 to 4;
T¹, T², T³ and T⁴ are each independently selected from (C₁-C₁₂)alkyl, substituted (C₁-C₁₂)alkyl, (EDA)_{w}, (PEG)ₙ, (AA)ₚ, —(CR¹³OH)ₕ—, piperidin-4-amino (4AP), an acetal group, a hydrazine, a disulfide, and an ester, wherein EDA is an ethylene diamine moiety, PEG is a polyethylene glycol or a modified polyethylene glycol, and AA is an amino acid residue, wherein w is an integer from 1 to 20, n is an integer from 1 to 30, p is an integer from 1 to 20, and h is an integer from 1 to 12;
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
W¹ is a drug; and
W² is the binding agent.

70. The conjugate of clause 69, wherein:
T¹ is selected from a (C₁-C₁₂)alkyl and a substituted (C₁-C₁₂)alkyl;
T², T³ and T⁴ are each independently selected from (EDA)_{w}, (PEG)ₙ, (C₁-C₁₂)alkyl, substituted (C₁-C₁₂)alkyl, (AA)ₚ, —(CR¹³OH)ₕ—, 4-amino-piperidine (4AP), an acetal group, a hydrazine, and an ester; and
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
   wherein:
(PEG)ₙ is

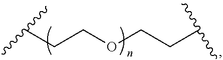

where n is an integer from 1 to 30;
EDA is an ethylene diamine moiety having the following structure:

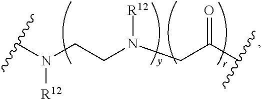

where y is an integer from 1 to 6 and r is 0 or 1;
4-amino-piperidine (4AP) is

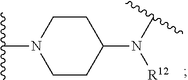

each R¹² and R¹⁵ is independently selected from hydrogen, an alkyl, a substituted alkyl, a polyethylene glycol moiety, an aryl and a substituted aryl, wherein any two adjacent R¹² groups may be cyclically linked to form a piperazinyl ring; and
R¹³ is selected from hydrogen, an alkyl, a substituted alkyl, an aryl, and a substituted aryl.

71. The conjugate of clause 69, wherein T¹, T², T³ and T⁴, and V¹, V², V³ and V⁴ are selected from the following table:

| T¹ | V¹ | T² | V² | T³ | V³ | T⁴ | V⁴ |
|---|---|---|---|---|---|---|---|
| (C₁-C₁₂)alkyl | —CONR¹⁵— | (PEG)ₙ | —CO— | — | — | — | — |
| (C₁-C₁₂)alkyl | —CO— | (AA)ₚ | —NR¹⁵— | (PEG)ₙ | —CO— | — | — |
| (C₁-C₁₂)alkyl | —CO— | (AA)ₚ | — | — | — | — | — |
| (C₁-C₁₂)alkyl | —CONR¹⁵— | (PEG)ₙ | —NR¹⁵— | — | — | — | — |
| (C₁-C₁₂)alkyl | —CO— | (AA)ₚ | —NR¹⁵— | (PEG)ₙ | —NR¹⁵— | — | — |
| (C₁-C₁₂)alkyl | —CO— | (EDA)_{w} | —CO— | — | — | — | — |
| (C₁-C₁₂)alkyl | —CONR¹⁵— | (C₁-C₁₂)alkyl | —NR¹⁵— | — | — | — | — |
| (C₁-C₁₂)alkyl | —CONR¹⁵— | (PEG)ₙ | —CO— | (EDA)_{w} | — | — | — |
| (C₁-C₁₂)alkyl | —CO— | (EDA)_{w} | — | — | — | — | — |
| (C₁-C₁₂)alkyl | —CO— | (EDA)_{w} | —CO— | (CR¹³OH)ₕ | —CONR¹⁵— | (C₁-C₁₂)alkyl | —CO— |
| (C₁-C₁₂)alkyl | —CO— | (AA)ₚ | —NR¹⁵— | (C₁-C₁₂)alkyl | —CO— | — | — |
| (C₁-C₁₂)alkyl | —CONR¹⁵— | (PEG)ₙ | —CO— | (AA)ₚ | — | — | — |
| (C₁-C₁₂)alkyl | —CO— | (EDA)_{w} | —CO— | (CR¹³OH)ₕ | —CO— | (AA)ₚ | — |
| (C₁-C₁₂)alkyl | —CO— | (AA)ₚ | —NR¹⁵— | (C₁-C₁₂)alkyl | —CO— | (AA)ₚ | — |
| (C₁-C₁₂)alkyl | —CO— | (AA)ₚ | —NR¹⁵— | (PEG)ₙ | —CO— | (AA)ₚ | — |
| (C₁-C₁₂)alkyl | —CO— | (AA)ₚ | —NR¹⁵— | (PEG)ₙ | —SO₂— | (AA)ₚ | — |
| (C₁-C₁₂)alkyl | —CO— | (EDA)_{w} | —CO— | (CR¹³OH)h | —CONR¹⁵— | (PEG)ₙ | —CO— |
| (C₁-C₁₂)alkyl | —CO— | (CR¹³OH)ₕ | —CO— | — | — | — | — |
| (C₁-C₁₂)alkyl | —CONR¹⁵— | substituted | —NR¹⁵— | (PEG)ₙ | —CO— | — | — |
| | | (C₁-C₁₂)alkyl | | | | | |
| (C₁-C₁₂)alkyl | —SO₂— | (C₁-C₁₂)alkyl | —CO— | — | — | — | — |
| (C₁-C₁₂)alkyl | —CONR¹⁵— | (C₁-C₁₂)alkyl | — | (CR¹³OH)ₕ | —CONR¹⁵— | — | — |
| (C₁-C₁₂)alkyl | —CO— | (AA)ₚ | —NR¹⁵— | (PEG)ₙ | —CO— | (AA)ₚ | —NR¹⁵— |
| (C₁-C₁₂)alkyl | —CO— | (AA)ₚ | —NR¹⁵— | (PEG)ₙ | —P(O)OH— | (AA)ₚ | — |
| (C₁-C₁₂)alkyl | —CO— | (EDA)_{w} | — | (AA)ₚ | — | — | — |
| (C₁-C₁₂)alkyl | —CONR¹⁵— | (C₁-C₁₂)alkyl | —NR¹⁵— | — | —CO— | — | — |
| (C₁-C₁₂)alkyl | —CONR¹⁵— | (C₁-C₁₂)alkyl | —NR¹⁵— | — | —CO— | (C₁-C₁₂)alkyl | —NR¹⁵— |
| (C₁-C₁₂)alkyl | —CO— | 4AP | —CO— | (C₁-C₁₂)alkyl | —CO— | (AA)ₚ | — |
| (C₁-C₁₂)alkyl | —CO— | 4AP | —CO— | (C₁-C₁₂)alkyl | —CO— | — | — |

72. The conjugate of any one of clauses 69 to 71, wherein the linker, L, is selected from one of the following structures:

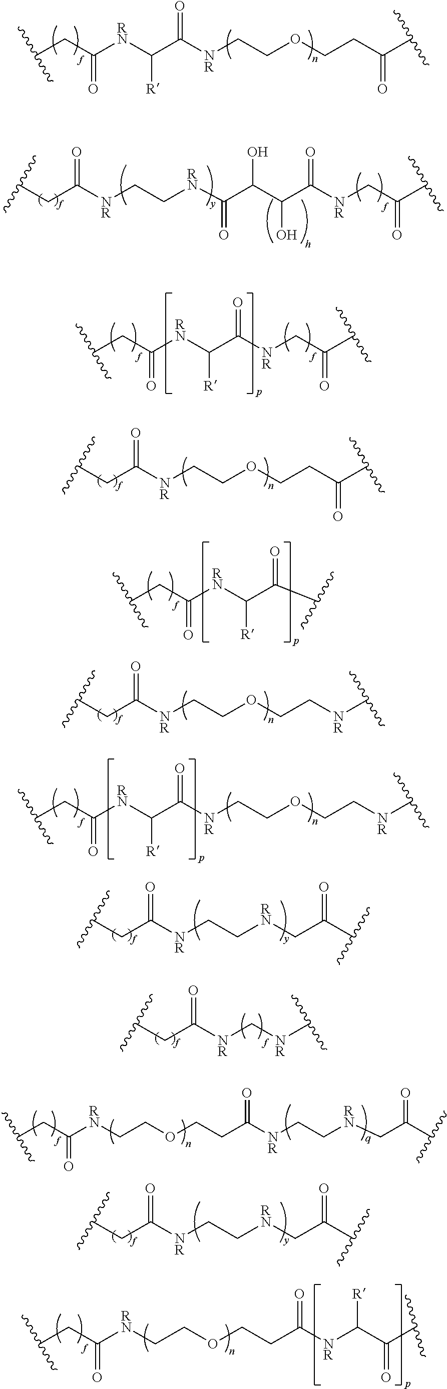

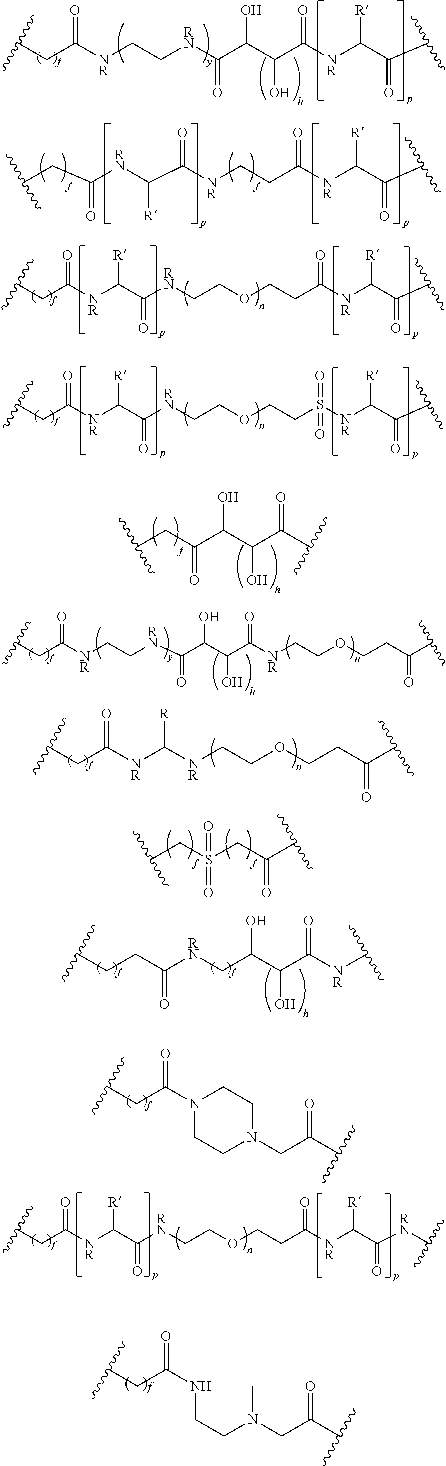

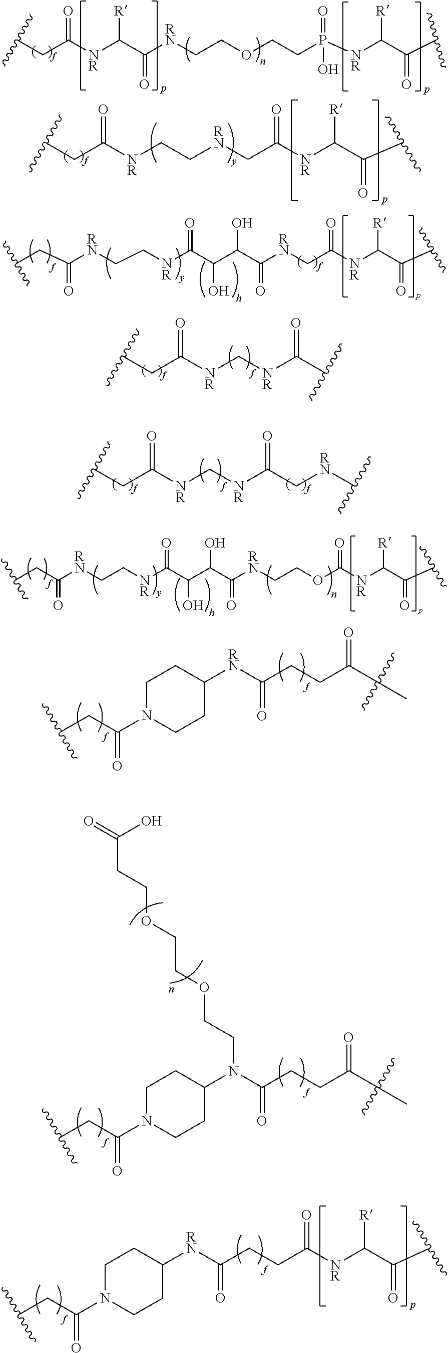

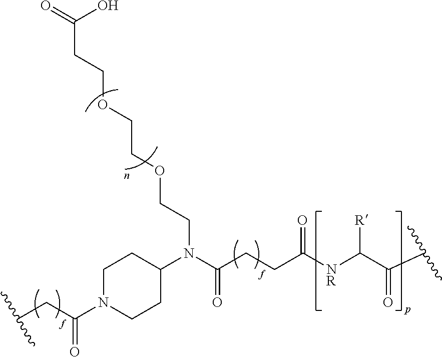

wherein
each f is independently 0 or an integer from 1 to 12;
each y is independently 0 or an integer from 1 to 20;
each n is independently 0 or an integer from 1 to 30;
each p is independently 0 or an integer from 1 to 20;
each h is independently 0 or an integer from 1 to 12;
each R is independently hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl; and
each R′ is independently H, a sidechain group of an amino acid, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

73. The conjugate of any one of clauses 69 to 72, wherein T¹ is (C₁-C₁₂)alkyl, V¹ is —CO—, T² is 4AP, V² is —CO—, T³ is (C₁-C₁₂)alkyl, V³ is —CO—, T⁴ is absent and V⁴ is absent.

74. The conjugate of any one of clauses 69 to 73, wherein the linker, L, comprises the following structure:

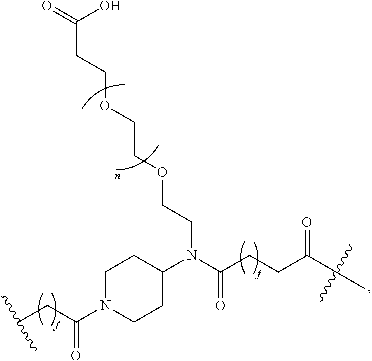

wherein
each f is independently an integer from 1 to 12; and
n is an integer from 1 to 30.

75. The conjugate of clause 60, wherein the conjugate is of formula (II):

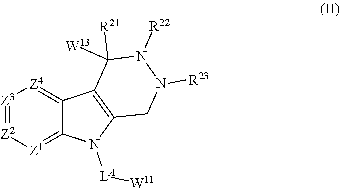

wherein:
Z¹, Z², Z³ and Z⁴ are each independently selected from CR²⁴, N and C-L^{B}-W¹², wherein at least one Z¹, Z², Z³ and Z⁴ is C-L^{B}-W¹²;
R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl;
each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹¹ is a first drug;
W¹² is a second drug; and
W¹³ is the binding agent.

76. The conjugate of clause 75, wherein Z¹ is CR²⁴.

77. The conjugate of clause 75, wherein Z¹ is N.

78. The conjugate of clause 75, wherein Z³ is C-L^{B}-W¹².

79. The conjugate of any of clauses 75 to 78, wherein L^{A} comprises:
-(T¹-V¹)ₐ-(T²-V²)_{b}-(T³-V³)_{c}-(T⁴-V⁴)_{d}-(T⁵-V⁵)ₑ-(T⁶-V⁶)_{f}—,
   wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

80. The conjugate of clause 79, wherein MABO, MABC, PABO, PABC, PAB, PABA, PAP and PHP are each optionally substituted with a glycoside.

81. The conjugate of clause 80, wherein the glycoside is selected from a glucuronide, a galactoside, a glucoside, a mannoside, a fucoside, O-GlcNAc, and O-GalNAc.

82. The conjugate of any of clauses 75 to 81,

wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

83. The conjugate of any of clauses 75 to 82, wherein L^{B} comprises:
-(T⁷-V⁷)_{g}-(T⁸-V⁸)ₕ-(T⁹-V⁹)-(T¹⁰-V¹⁰)ⱼ-(T¹¹-V¹¹)ₖ-(T¹²-V¹²)ᵢ-(T¹³-V¹³)ₘ,
   wherein
g, h, i, j, k, l and m are each independently 0 or 1;
T⁷, T⁸, T⁹, T¹⁰, T⁴, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each
q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

84. The conjugate of clause 83, wherein MABO, MABC, PABO, PABC, PAB, PABA, PAP and PHP are each optionally substituted with a glycoside.

85. The conjugate of any of clauses 83 to 84, wherein the glycoside is selected from a glucuronide, a galactoside, a glucoside, a mannoside, a fucoside, O-GlcNAc, and O-GalNAc.

86. The conjugate of any of clauses 83 to 85,

wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0.

87. The conjugate of clause 75, wherein the conjugate has the structure:

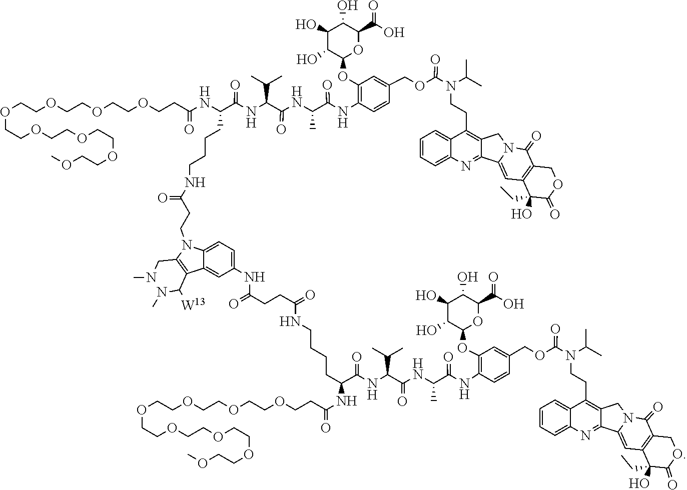

88. A pharmaceutical composition comprising:
a) the binding agent of any one of clauses 1-47; and
b) a pharmaceutically acceptable carrier.

89. A pharmaceutical composition comprising:
a) the fusion protein of clause 58; and
b) a pharmaceutically acceptable carrier.

90. A pharmaceutical composition comprising:
a) the conjugate of any one of clauses 59-87; and
b) a pharmaceutically acceptable carrier.

91. The pharmaceutical composition of any one of clauses 88 to 90, further comprising a T cell activator.

92. The pharmaceutical composition of clause 91, wherein the T cell activator is selected from the group consisting of: an immune checkpoint inhibitor, a cytokine, and an antagonist of an inhibitory immune receptor.

93. The pharmaceutical composition of any one of clauses 88 to 92, wherein the antibody is encapsulated in a liposome.

94. A method of treating a cell proliferative disorder in a subject, the method comprising:
administering to a subject having a cell proliferative disorder a therapeutically effective amount of the pharmaceutical composition of any one of clauses 88 to 93.

95. The method of clause 94, further comprising administering to the subject a therapeutically effective amount of an immunomodulatory therapeutic agent.

96. The method of clause 95, wherein the immunomodulatory therapeutic agent is an immune checkpoint inhibitor or interleukin.

97. The method of clause 96, wherein the immune checkpoint inhibitor inhibits A2AR, B7-H3, B7-H4, BTLA, CTLA-4, CD277, IDO, KIR, PD-1, LAG-3, TIM-3, TIGIT and VISTA.

98. The method of clause 97, wherein the immune checkpoint inhibitor inhibits PD-1 signaling.

99. The method of clause 98, wherein immune checkpoint inhibitor that inhibits PD-1 signaling is an anti-PD-1 antibody.

100. The method of clause 99, wherein the anti-PD-1 antibody is nivolumab, pembrolizumab, atezolizumab, durvalumab, or avelumab.

101. The method of clause 97, wherein the immune checkpoint inhibitor inhibits CTLA-4.

102. The method of clause 101, wherein the inhibitor of CTLA-4 is an anti-CTLA-4 antibody.

103. The method of clause 102, wherein the anti-CTLA-4 antibody is ipilimumab.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

Commercially available reagents referred to in the Examples were used according to manufacturer's instructions unless otherwise indicated. Unless otherwise defined, all technical and scientific terms used herein have the same meaning as commonly understood by one of ordinary skill in the art to which this invention belongs. Exemplary methods and materials are described below although methods and materials similar or equivalent to those described herein can also be used in the practice or testing of the present invention. The materials, methods, and examples are illustrative only and not intended to be limiting in scope.

### Example 1: Generation of Variants of AC-10

Twenty-five variants of AC-10 were generated based on CDR grafting of the chimeric sequences onto framework regions bearing different levels of human germline-like similarity. Specifically, five heavy and five light chain sequence variants were designed where the framework regions ranged from 83.5% to 100% human germline sequence. Each heavy and light chain was tested in combination with each other for a total of 25 variants analyzed. Of these, two heavy chain variants (VH2 and VH3) when tested in combination with any light chain sequence yielded antibodies that no longer bound to CD30 antigen, eliminating 10 antibody variants from selection. The remaining 15 antibodies, representing combined heavy and light chain framework regions that ranged between 85 and 98.9% human germline-like, were prepared as ADCs and tested for in vitro potency (Tables 4-6). From these data, the most potent humanized variants were taken forward for additional testing, including stability testing and in vivo efficacy as ADCs.

### Example 2

A linker-payload (RED-106) containing a 4-amino-piperidine (4AP) group was synthesized according to Scheme 1, shown below.

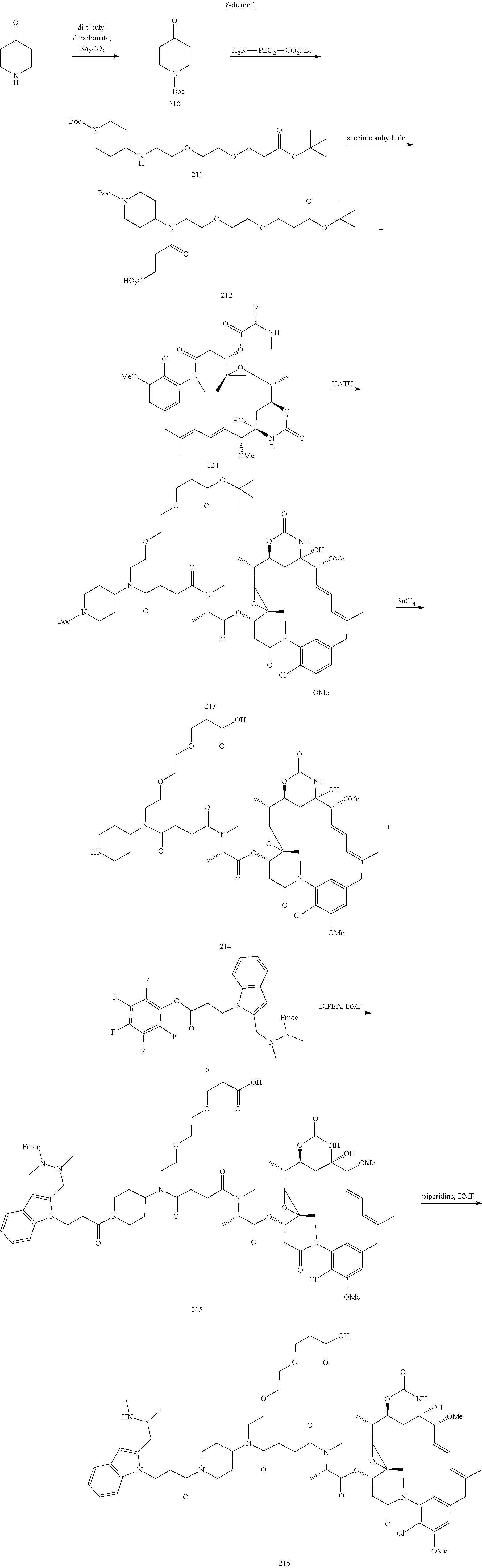

### Synthesis of tert-butyl 4-oxopiperidine-1-carboxylate (210)

To a 100 mL round-bottom flask containing a magnetic stir bar was added piperidin-4-one hydrochloride monohydrate (1.53 g, 10 mmol), di-tert-butyl dicarbonate (2.39 g, 11 mmol), sodium carbonate (1.22 g, 11.5 mmol), dioxane (10 mL), and water (1 mL). The reaction mixture was stirred at room temperature for 1 h. The mixture was diluted with water (100 mL) and extracted with EtOAc (3×100 mL). The combined organic layers were washed with brine, dried over Na₂SO₄, filtered, and concentrated under reduced pressure. The resulting material was dried in vacuo to yield 1.74 g (87%) of compound 210 as a white solid.

¹H NMR (CDCl₃) δ 3.73 (t, 4H, J=6.0), 2.46 (t, 4H, J=6.0), 1.51 (s, 9H).

MS (ESI) m/z: [M+H]⁺ Calcd for C₁₀H₁₈NO₃ 200.3; Found 200.2.

### Synthesis of tert-butyl 4-((2-(2-(3-(tert-butoxy)-3-oxopropoxy)ethoxy)ethyl)amino)piperidine-1-carboxylate (211)

To a dried scintillation vial containing a magnetic stir bar was added tert-butyl 4-oxopiperidine-1-carboxylate (399 mg, 2 mmol), H₂N-PEG₂-COOt-Bu (550 mg, 2.4 mmol), 4 Å molecular sieves (activated powder, 200 mg), and 1,2-dichloroethane (5 mL). The mixture was stirred for 1 h at room temperature. To the reaction mixture was added sodium triacetoxyborohydride (845 mg, 4 mmol). The mixture was stirred for 3 days at room temperature. The resulting mixture was partitioned between EtOAc and saturated aqueous NaHCO₃. The organic layer was washed with brine, dried over Na₂SO₄, filtered, and concentrated under reduced pressure to afford 850 mg of compound 211 as a viscous oil.

MS (ESI) m/z: [M+H]⁺ Calcd for C₂₁H₄₁N₂O₆ 417.3; Found 417.2.

### Synthesis of 13-(1-(tert-butoxycarbonyl)piperidin-4-yl)-2,2-dimethyl-4,14-dioxo-3,7,10-trioxa-13-azaheptadecan-17-oic acid (212)

To a dried scintillation vial containing a magnetic stir bar was added tert-butyl 4-((2-(2-(3-(tert-butoxy)-3-oxopropoxy)ethoxy)ethyl)amino)piperidine-1-carboxylate 211 (220 mg, 0.5 mmol), succinic anhydride (55 mg, 0.55 mmol), 4-(dimethylamino)pyridine (5 mg, 0.04 mmol), and dichloromethane (3 mL). The mixture was stirred for 24 h at room temperature. The reaction mixture was partially purified by flash chromatography (elute 50-100% EtOAc/hexanes) to yield 117 mg of compound 212 as a clear oil, which was carried forward without further characterization.

MS (ESI) m/z: [M+H]⁺ Calcd for C₂₅H₄₅N₂O₉ 517.6; Found 517.5.

### Synthesis of 17-(tert-butyl) 1-((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl) (2S)-8-(1-(tert-butoxycarbonyl)piperidin-4-yl)-2,3-dimethyl-4,7-dioxo-11,14-dioxa-3,8-diazaheptadecanedioate (213)

To a dried scintillation vial containing a magnetic stir bar was added 13-(1-(tert-butoxycarbonyl)piperidin-4-yl)-2,2-dimethyl-4,14-dioxo-3,7,10-trioxa-13-azaheptadecan-17-oic acid 212 (55 mg, 0.1 mmol), N-deacyl maytansine 124 (65 mg, 0.1 mmol), HATU (43 mg, 0.11 mmol), DMF (1 mL), and dichloromethane (0.5 mL). The mixture was stirred for 8 h at room temperature. The reaction mixture was directly purified by C18 flash chromatography (elute 5-100% MeCN/water) to give 18 mg (16%) of compound 213 as a white film.

MS (ESI) m/z: [M+H]⁺ Calcd for C₅₇H₈₇ClN₅O₁₇ 1148.6; Found 1148.7.

### Synthesis of (2S)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-2,3-dimethyl-1,4,7-trioxo-8-(piperidin-4-yl)-11,14-dioxa-3,8-diazaheptadecan-17-oic acid (214)

To a dried scintillation vial containing a magnetic stir bar was added maytansinoid 213 (31 mg, 0.027 mmol) and dichloromethane (1 mL). The solution was cooled to 0° C. and tin(IV) tetrachloride (1.0 M solution in dichloromethane, 0.3 mL, 0.3 mmol) was added. The reaction mixture was stirred for 1 h at 0° C. The reaction mixture was directly purified by C18 flash chromatography (elute 5-100% MeCN/water) to yield 16 mg (60%) of compound 214 as a white solid (16 mg, 60% yield).

MS (ESI) m/z: [M+H]⁺ Calcd for C₄₈H₇₁ClN₅O₁₅ 992.5; Found 992.6.

### Synthesis of (2S)-8-(1-(3-(2-((2-(((9H-fluoren-9-yl)methoxy)carbonyl)-1,2-dimethylhydrazinyl)methyl)-1H-indol-1-yl)propanoyl)piperidin-4-yl)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-2,3-dimethyl-1,4,7-trioxo-11,14-dioxa-3,8-diazaheptadecan-17-oic acid (215)

To a dried scintillation vial containing a magnetic stir bar was added maytansinoid 214 (16 mg, 0.016 mmol), (9H-fluoren-9-yl)methyl 1,2-dimethyl-2-((1-(3-oxo-3-(perfluorophenoxy)propyl)-1H-indol-2-yl)methyl)hydrazine-1-carboxylate (5) (13 mg, 0.02 mmol), DIPEA (8 μL, 0.05 mmol), and DMF (1 mL). The solution was stirred for 18 h at room temperature. The reaction mixture was directly purified by C18 flash chromatography (elute 5-100% MeCN/water) to yield 18 mg (77%) of compound 215 as a white solid.

MS (ESI) m/z: [M+H]⁺ Calcd for C₇₇H₉₈ClN₈O₁₈ 1457.7; Found 1457.9.

### Synthesis of (2S)-1-(((1⁴S,1⁶S,3³S,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-8-(1-(3-(2-((1,2-dimethylhydrazinyl)methyl)-1H-indol-1-yl)propanoyl)piperidin-4-yl)-2,3-dimethyl-1,4,7-trioxo-11,14-dioxa-3,8-diazaheptadecan-17-oic acid (216)

To a dried scintillation vial containing a magnetic stir bar was added maytansinoid 215 (18 mg, 0.012 mmol), piperidine (20 μL, 0.02 mmol), and DMF (1 mL). The solution was stirred for 20 minutes at room temperature. The reaction mixture was directly purified by C18 flash chromatography (elute 1-60% MeCN/water) to yield 15 mg (98%) of compound 216 (also referred to herein as HIPS-4AP-maytansine or HIPS-4-amino-piperidin-maytansine) as a white solid.

MS (ESI) m/z: [M+H]⁺ Calcd for C₆₂H₈₈ClN₈O₁₆ 1235.6; Found 1236.0.

### Example 3: Bioconjugation, Purification, and HPLC Analysis of ADCs

Antibodies (15 mg/mL) bearing one or two aldehyde tags (single or double-tagged constructs, FIGS. 1A-IC) were conjugated to linker-payloads at 1.1 or 1.7 mM, respectively. Reactions proceeded for 72 h at 37° C. in 20 mM sodium citrate, 50 mM NaCl pH 5.5 (20/50 buffer) containing 0.85-2.5% DMA. In some cases, Triton-X-100 was added to 0.25% to improve linker-payload solubility. After conjugation, free drug was removed using a 30 kD MWCO 0.5 mL Amicon spin concentrator. Samples were added to the spin concentrator, centrifuged at 15,000×g for 7 min, then diluted with 450 μL 20 mM sodium citrate, 50 mM NaCl pH 5.5, and centrifuged again. The process was repeated 10 times. To determine the drug-antibody ratio (DAR) of the final product, antibody-drug conjugates (ADCs) were examined by analytical chromatography using HIC (Tosoh #14947) or PLRP-RP (Agilent PL1912-1802 1000A, 8 μm, 50×2.1 mm) columns. HIC analysis used mobile phase A: 1.5 M ammonium sulfate, 25 mM sodium phosphate pH 7.0, and mobile phase B: 25% isopropanol, 18.75 mM sodium phosphate pH 7.0. PLRP analysis used mobile phase A: 0.1% trifluoroacetic acid in water, and mobile phase B: 0.1% trifluoroacetic acid in acetonitrile. Prior to PLRP analysis, sample was denatured with the addition of 50 mM DTT, 4 M guanidine HCl (final concentrations) and heating at 37° C. for 30 min. To determine aggregation, samples were analyzed using analytical size exclusion chromatography (SEC; Tosoh #08541) with a mobile phase of 300 mM NaCl, 25 mM sodium phosphate pH 6.8 with 5% isopropanol.

ADCs were conjugated at the antibody heavy chain C-terminus to a non-cleavable linker bearing a maytansine payload (RED-106) for a DAR of ˜1.9.

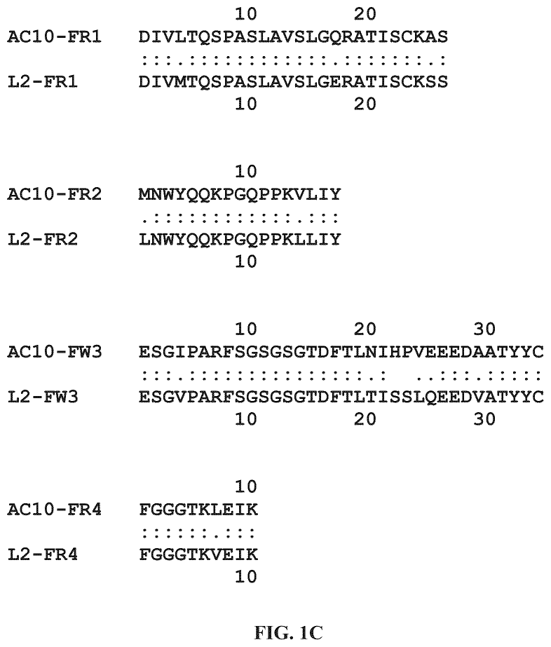

FIG. 3. CT-tagged H1/L1 antibody conjugated to a noncleavable linker bearing a maytansine payload (RED-106) yields a DAR of 1.88 as determined by hydrophobic interaction chromatography (HIC).

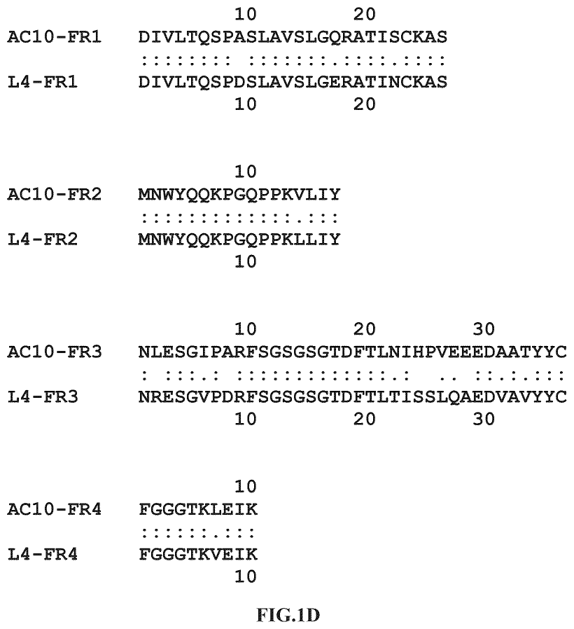

FIG. 4. CT-tagged H1/L1 antibody conjugated to RED-106 is 99.2% monomeric as determined by size-exclusion chromatography (SEC).

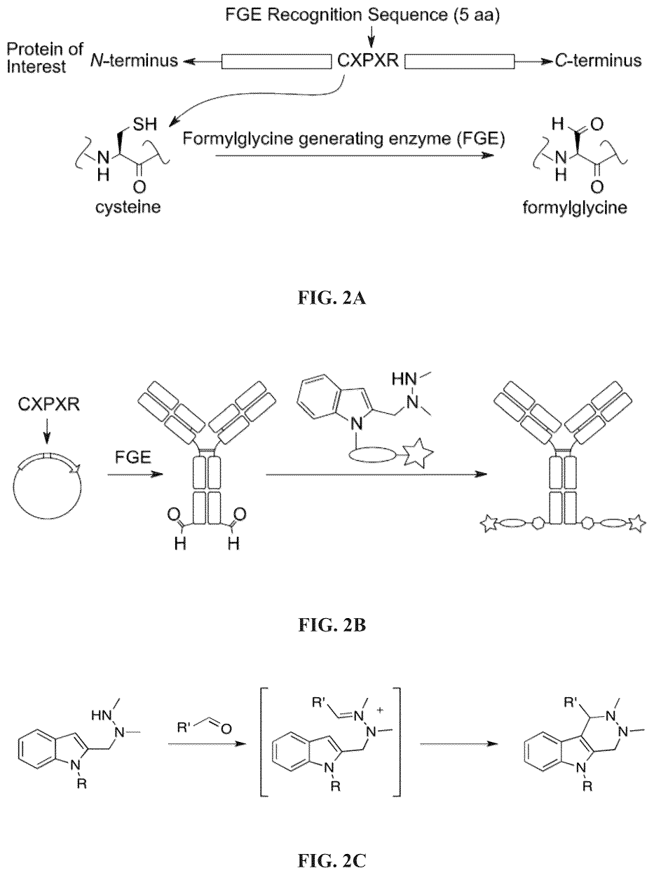

FIG. 5. CT-tagged H1/L4 antibody conjugated to RED-106 yields a DAR of 1.91 as determined by HIC.

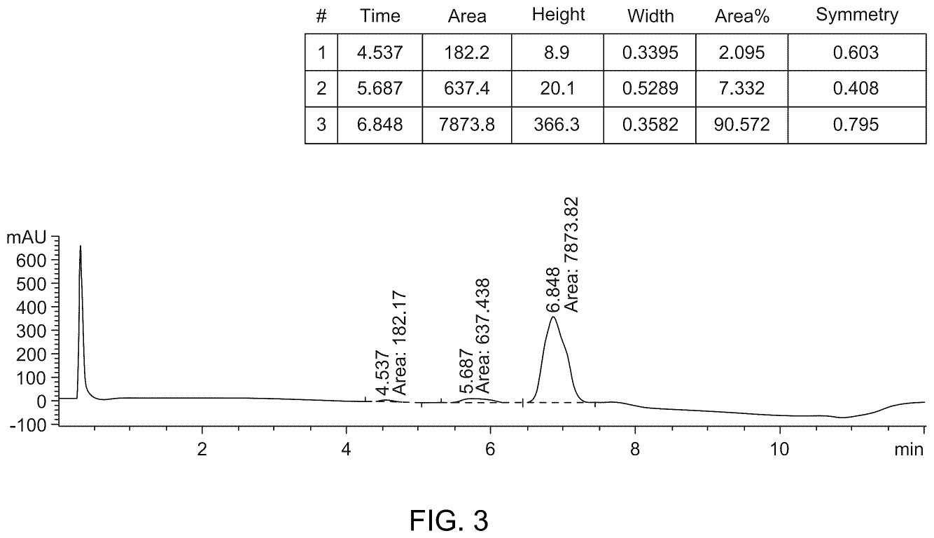

FIG. 6. CT-tagged H1/L4 antibody conjugated to RED-106 is 99.5% monomeric as determined by SEC.

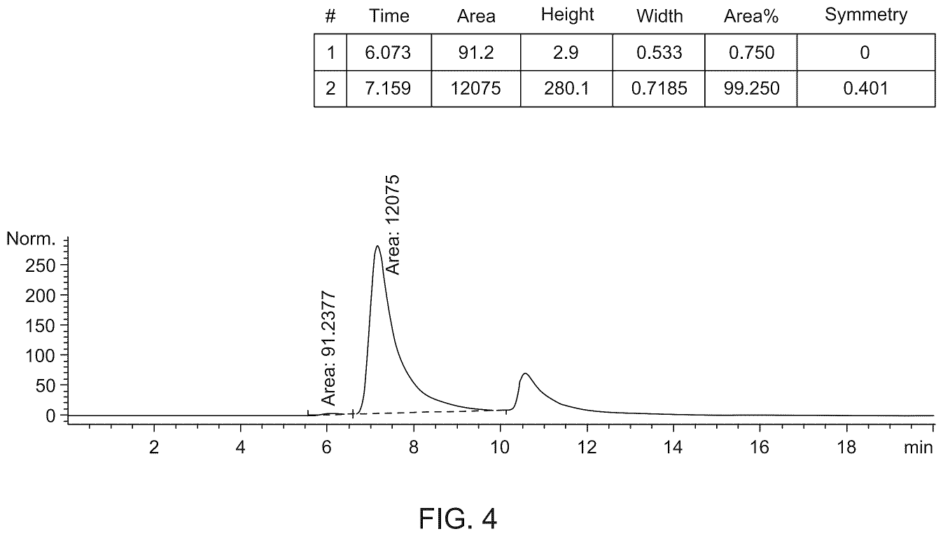

FIG. 7. CT-tagged H4/L2 antibody conjugated to RED-106 yields a DAR of 1.89 as determined by HIC.

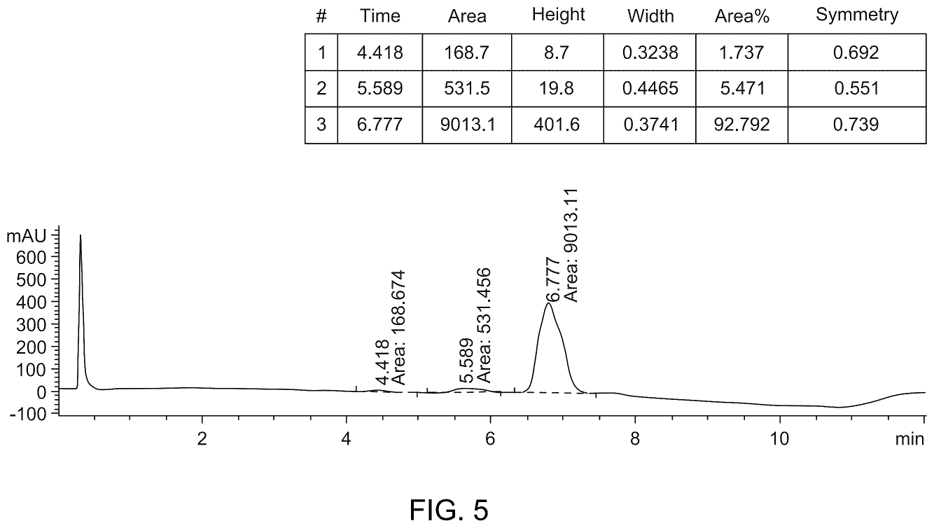

FIG. 8. CT-tagged H4/L2 antibody conjugated to RED-106 is 99.7% monomeric as determined by SEC.

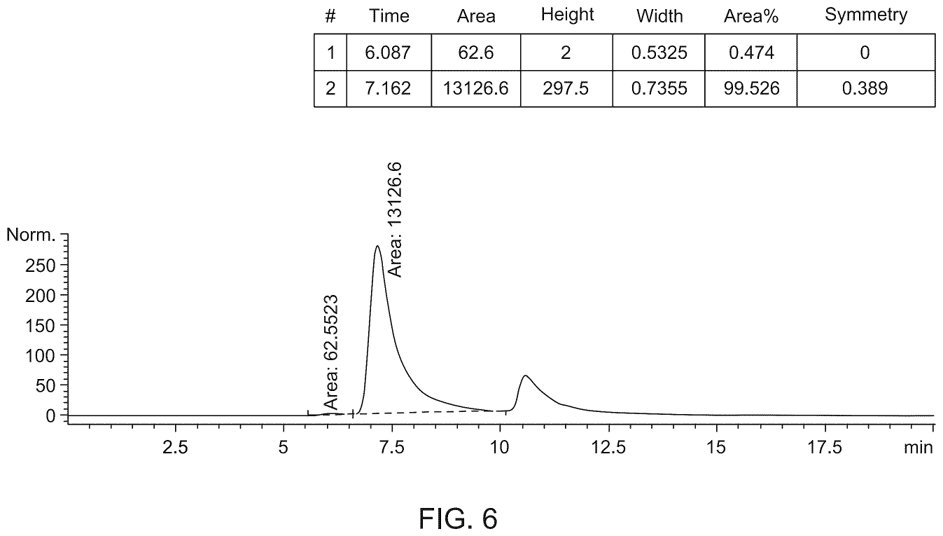

FIG. 9. CT-tagged H4/L4 antibody conjugated to RED-106 yields a DAR of 1.90 as determined by HIC.

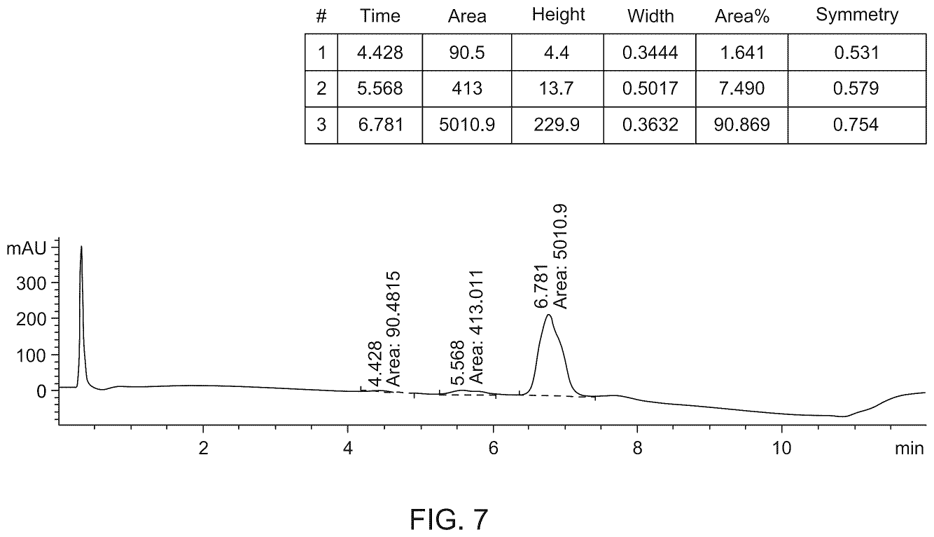

FIG. 10. CT-tagged H4/L4 antibody conjugated to RED-106 is 99.5% monomeric as determined by SEC.

### Example 4: Binding of Humanized Anti-CD30 Antibodies to Recombinant CD30

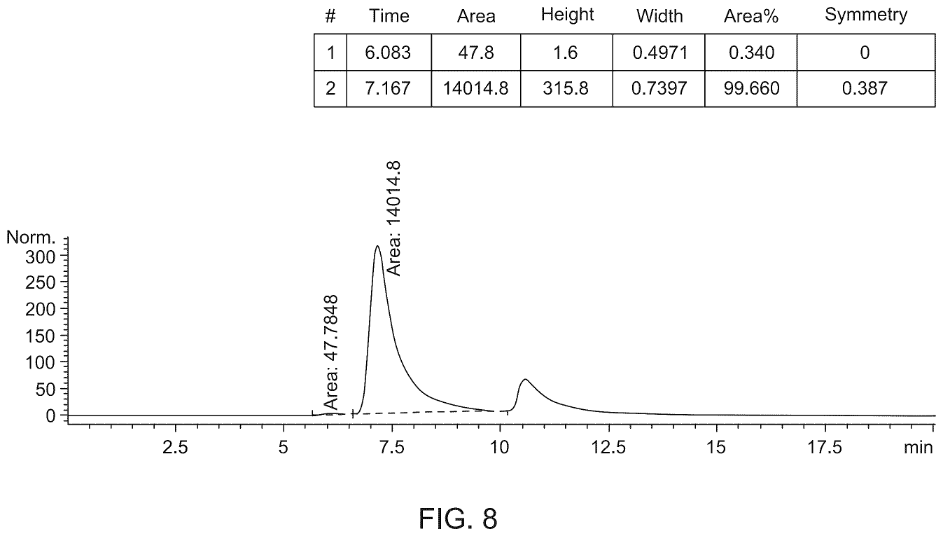

ELISA was performed to estimate binding of anti-CD30 antibodies comprising framework regions from the VH and VL chains described in this disclosure. Binding to CD30 was not affected in various combinations of tested variants containing H1 variant combined with several light chain variants L1 to L5 (FIG. 11).

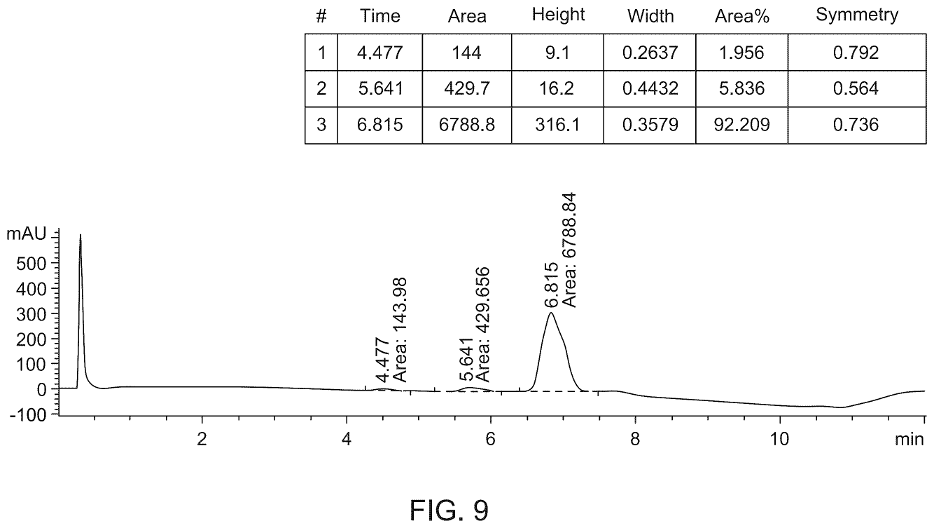

Similarly, ELISA was performed to estimate binding of anti-CD30 antibodies comprising framework regions from the VH and VL chains described in this disclosure. Binding to CD30 was not affected in various combinations of tested variants containing H4 variant combined with several light chain variants L1 to L5 (FIG. 12).

### Example 5: In Vitro Cytotoxicity of Anti-CD30 ADCs Against CD30-Expressing

| | | SUDHL1 | L540 |
|---|---|---|---|
| CD30 H1/L1 | cRW2841 | 0.298 | 0.408 |

| | | SUDHL1 | L540 |
|---|---|---|---|
| CD30 H4/L1 | cRW2856 | 0.201 | 0.518 |
| CD30 H4/L2 | cRW2857 | 0.366 | 0.728 |
| CD30 H4/L3 | cRW2858 | 17.310 | 5.120 |
| CD30 H4/L4 | cRW2859 | 0.040 | 0.466 |
| CD30 H4/L5 | cRW2860 | 1.810 | 0.500 |
| CD30 Original | cRW2866 | 0.203 | 0.661 |

TABLE 6
IC50 values reflecting the in vitro potency of
humanized C30-targeted maytansine-conjugated
ADCs comprising the antibody H5 heavy chain.

| | | SUDHL1 | L540 |
|---|---|---|---|
| CD30 H5/L1 | cRW2861 | 0.528 | 0.465 |
| CD30 H5/L2 | cRW2862 | 0.484 | 0.668 |
| CD30 H5/L3 | cRW2863 | 21.23 | 3.855 |
| CD30 H5/L4 | cRW2864 | 0.01 | 0.407 |
| CD30 H5/L5 | cRW2865 | 7.49 | 0.533 |
| CD30 Original | cRW2866 | 0.203 | 0.661 |

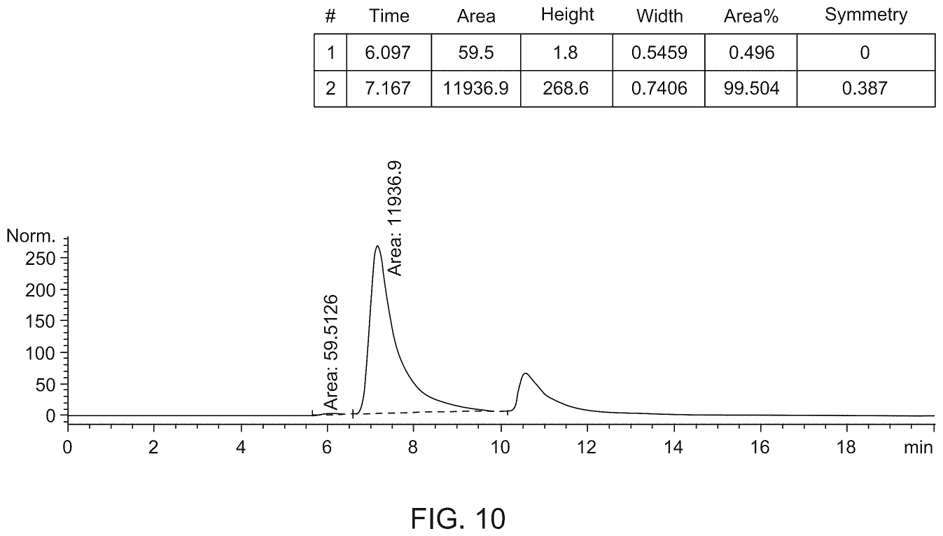

In vitro cytotoxicity assessment was performed for anti-CD30 antibodies comprising framework regions from the VH and VL chains described in this disclosure. Some of the tested variants containing H1 variant combined with several light chain variants L1 to L5 exhibited desirable cytotoxicity against SU-DHL-1 cells (FIG. 13).

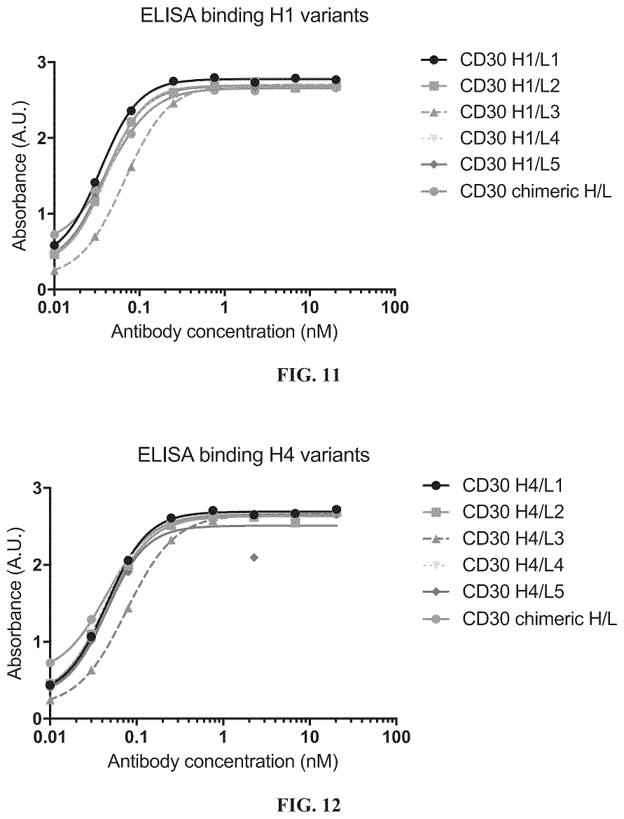

In vitro cytotoxicity assessment was performed for anti-CD30 antibodies comprising framework regions from the VH and VL chains described in this disclosure. Some of the tested variants containing H1 variant combined with several light chain variants L1 to L5 exhibited desirable cytotoxicity against L540 cells (FIG. 14).

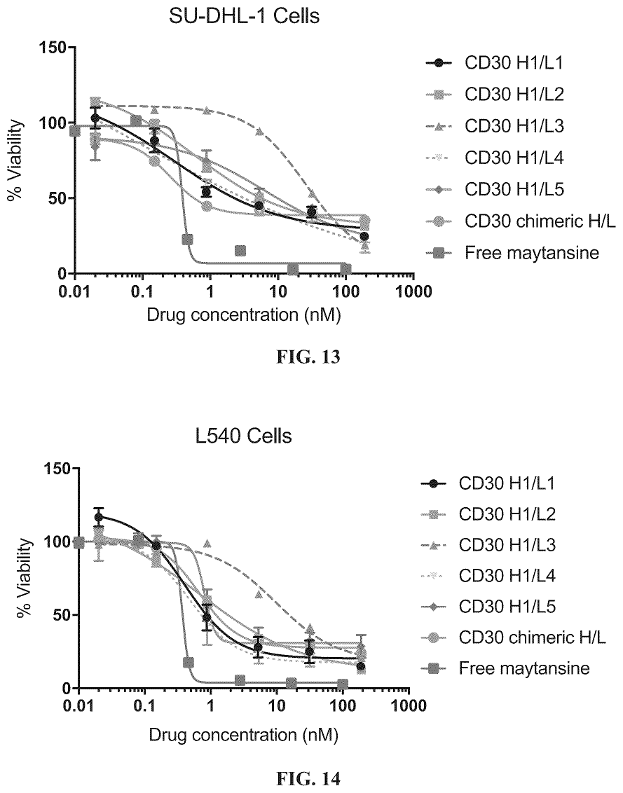

In vitro cytotoxicity assessment was performed for anti-CD30 antibodies comprising framework regions from the VH and VL chains described in this disclosure. Some of the tested variants containing H4 variant combined with several light chain variants L1 to L5 exhibited desirable cytotoxicity against SU-DHL-1 cells (FIG. 15).

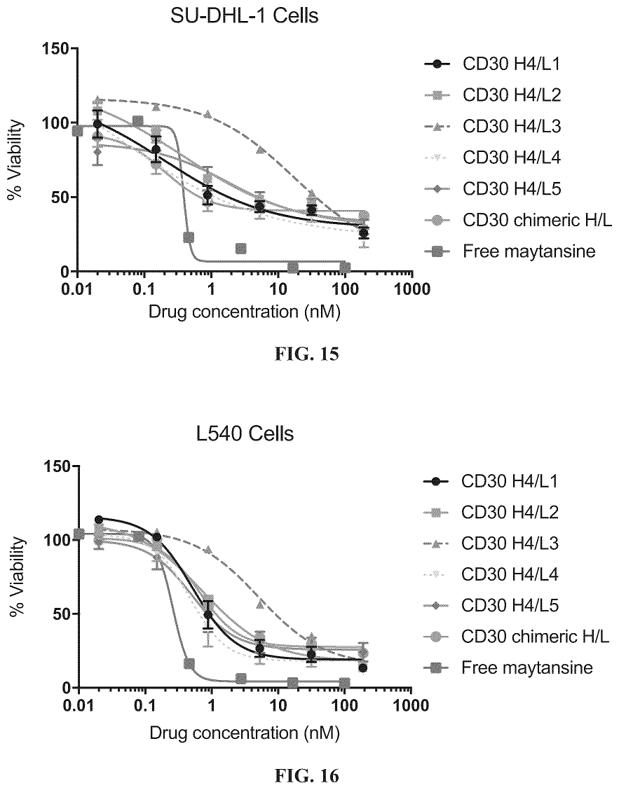

In vitro cytotoxicity assessment was performed for anti-CD30 antibodies comprising framework regions from the VH and VL chains described in this disclosure. Some of the tested variants containing H4 variant combined with several light chain variants L1 to L5 exhibited desirable cytotoxicity against L540 cells (FIG. 16).

### Example 6: Biophysical Characterization—Stability

Melting temperature and forced stability results showed that the humanized variants exhibited improved characteristics relative to the parental chimera. For example, Table 7 below shows that humanized antibody and ADC melting temperatures as determined by differential scanning fluorimetry show improvements over chimeric sequence.

TABLE 7
Melting temperatures of humanized antibody and ADC

| Antibody | Fab Tm (° C.) | ADC | Fab Tm (° C.) |
|---|---|---|---|
| H1/L1 | 74.35 | H1/L1 | 72 |
| H1/L4 | 81.17 | H1/L4 | 80 |
| H4/L2 | 75.54 | H4/L2 | 73 |
| H4/L4 | 80.43 | H4/L4 | 82 |
| H5/L1 | 71.39 | H5/L1 | 70 |
| H5/L2 | 71.09 | H5/L2 | 71 |
| H5/L4 | 73.61 | H5/L4 | 73 |
| Chimeric H/L | 78.3 | | |

ADCs were conjugated at the antibody heavy chain C-terminus to a noncleavable linker bearing a maytansine payload for a DAR of ˜1.9.

TABLE 8
Humanized ADC forced stability at pH 5.5 and 7.2
shows improvements over chimeric sequence.

| | HWM pH 5.5, | HWM pH 5.5, | HWM pH 7.2, | HWM pH 7.2, |
|---|---|---|---|---|
| ADC | 3 d −80° C. | 3 d 45° C. | 3 d −80° C. | 3 d 45° C. |
| H1/L1 | 2.18% | 17.36% | 3.67% | 16.59% |
| H1/L4 | 1.66% | 10.05% | 2.90% | 16.42% |
| H4/L2 | 1.24% | 6.18% | 2.37% | 30.38% |
| H4/L4 | 1.66% | 7.70% | 3.14% | 23.28% |
| H5/L1 | 1.12% | 7.60% | 2.50% | 18.37% |
| H5/L2 | 1.53% | 7.56% | 3.06% | 29.09% |
| H5/L4 | 1.59% | 8.47% | 2.81% | 36.33% |
| Chimeric H/L | 1.24% | 20.78% | 2.60% | 41.64% |

ADCs were conjugated at the antibody heavy chain C-terminus to a noncleavable linker bearing a maytansine payload for a DAR of ˜1.9. Samples were placed in buffer at the designated pH and temperature for 3 days, then analyzed by size-exclusion chromatography. HMW, high-molecular weight species.

### Example 7: In Vivo Efficacy of Anti-CD30 ADCs Against a HuT102 Xenograft Model

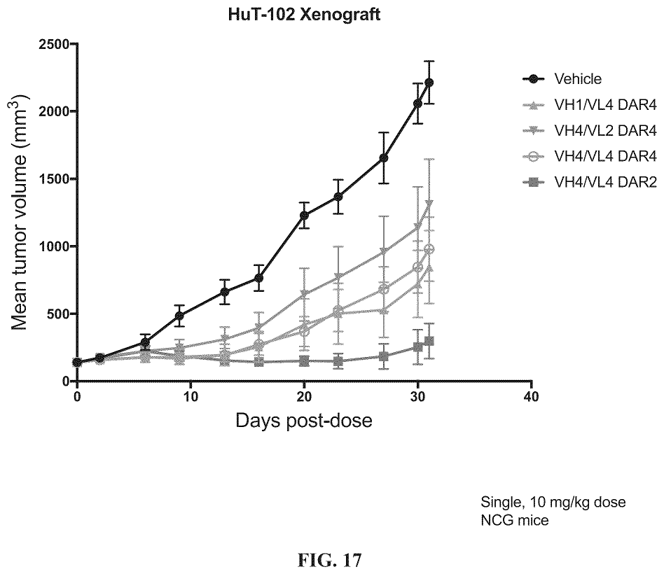

Methods: Female NCG mice (8 per group) were inoculated subcutaneously with HuT102 cells. Treatment began when the tumors reached an average of 139 mm³, at which time the animals were dosed intravenously with vehicle alone or a single dose of the anti-CD30 ADC at 10 mg/kg. Anti-CD30 ADCs carrying two aldehyde tags (CH1/CT) or one aldehyde tag (CT) and conjugated to a noncleavable linker bearing a maytansine payload (RED-106) to yield drug-to-antibody ratios of ˜4 or ˜2, respectively, were tested. The animals were monitored twice weekly for body weight and tumor size. Animals were euthanized when tumors reached 2000 mm³. All of the DAR4 ADCs were efficacious against the HuT 102 tumors, delaying tumor growth for about 2 wk post-dose. The DAR2 ADC was even more efficacious, preventing tumor growth for the duration of the study (FIG. 17).

### Example 8

### Synthesis of MMAE Construct 8

Compounds 1 and 4 were obtained commercially from Shanghai Medicilon and used as received. Monomethylauristatin A 5 (MMAE) was purchased from BroadPharm. All other reagents were obtained from commercial sources and used without purification.

### Preparation of (R)-2-(3-(2-((2-(((9H-fluoren-9-yl)methoxy)carbonyl)-1,2-dimethylhydrazineyl)methyl)-1H-pyrrolo[2,3-b]pyridin-1-yl)propanamido)-3-oxo-3-((2-(2-(3-oxo-3-(perfluorophenoxy)propoxy)ethoxy)ethyl)amino)propane-1-sulfonic acid (3)

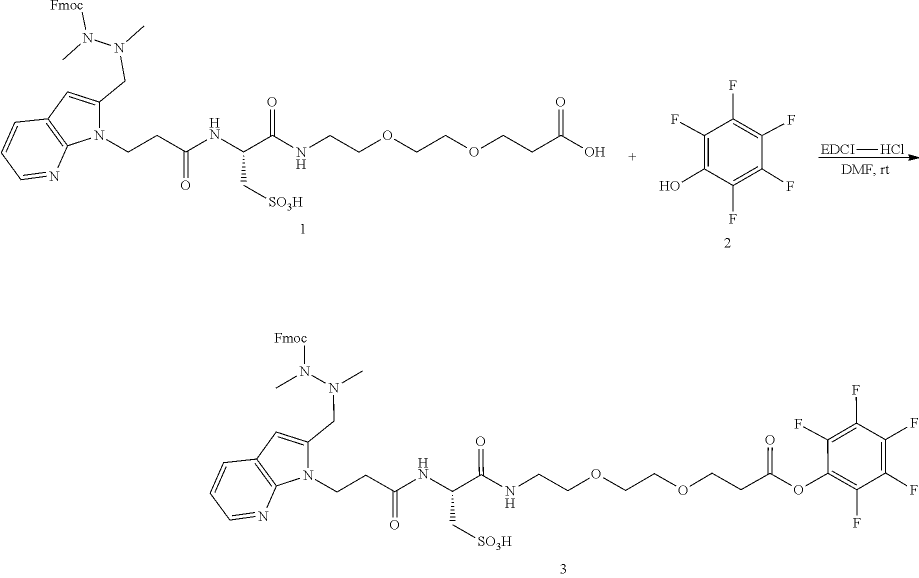

Carboxylic acid 1 (1.33 g, 1.67 mmol) was combined with pentafluorophenol 2 (1.23 g, 6.68 mmol) in 6.5 mL of anhydrous DMF. This mixture was treated with EDCI-HCl (0.64 g, 3.34 mmol) in one portion at room temperature and stirred for 20 h until 1 was fully consumed as judged by HPLC analysis. Reaction mixture was directly purified by reversed-phase chromatography (C18 column, 0-80% acetonitrile-water with 0.05% TFA). Pure fractions were combined, concentrated under vacuum until murky, and lyophilized to give PFP-ester product 3 (1.40 g, 1.46 mmol, 87% yield) as a tan powder. LRMS (ESI): m/z 961.2 [M+H]+, Calcd for C₄₄H₄₅F₅N₆O₁₁S m/z 961.3.

### Preparation of (2S,3R,4S,5S,6S)-2-(2-((S)-2-((S)-2-((((9H-fluoren-9-yl)methoxy)carbonyl)amino)-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (6)

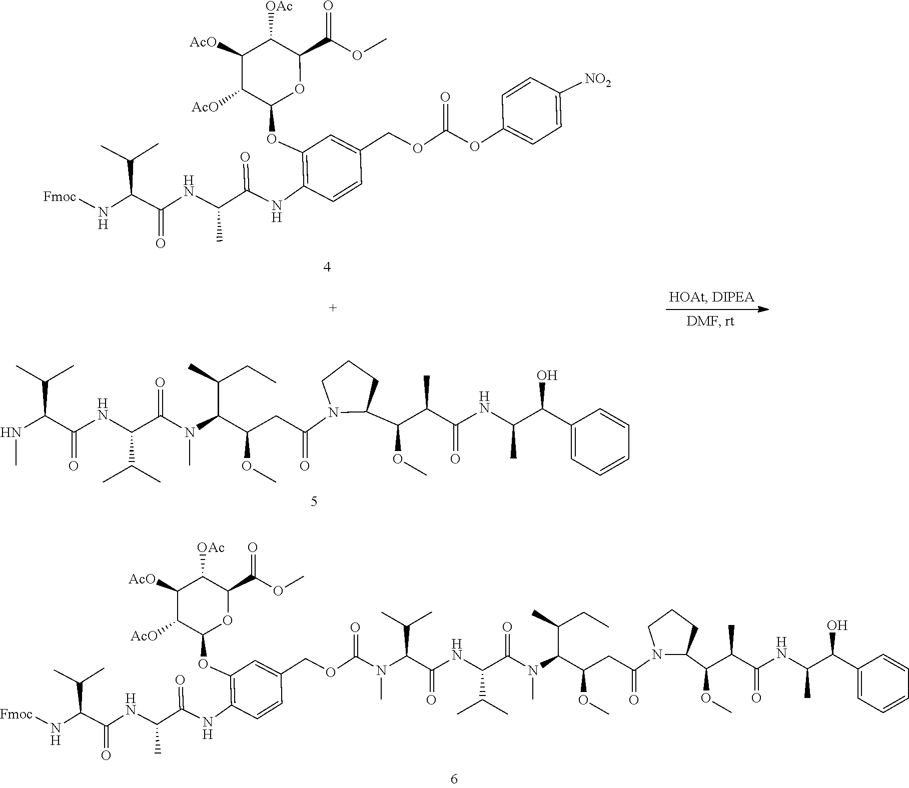

In a 20 mL glass vial were combined monomethyl auristatin A 5 (720 mg, 1.0 mmol), 5 mL of anhydrous DMF, and 0.35 mL of DIPEA (2.0 mmol) at room temperature. The resulting mixture was stirred and treated with PNP carbonate 4 (1014 mg, 1.0 mmol) as a solid in a few small portions, followed by the addition of HOAt (136 mg, 1.0 mmol) in one portion at room temperature. Reaction mixture was stirred for 6 h until reaction was judged complete (HPLC). Reaction mixture was poured into 30 mL of water, and the resulting precipitate was separated by spinning and collected, washed with 5 mL of water, and dried briefly under high vacuum to give 1.87 g of crude product 6 as a yellowish solid, which was taken to the next step without purification.

### Preparation of (2S,3S,4S,5R,6S)-6-(2-((S)-2-((S)-2-amino-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (7)

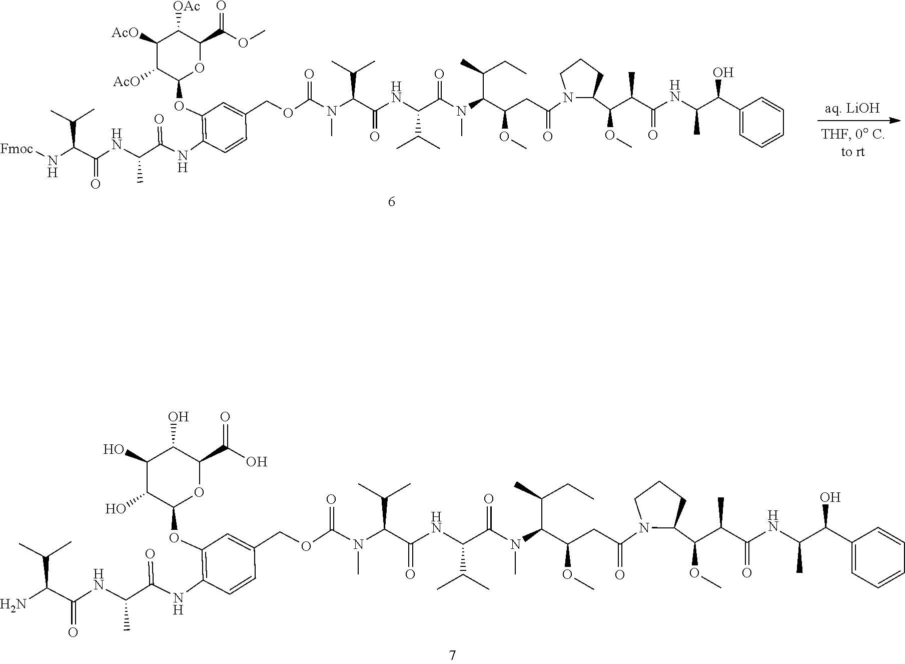

A solution of crude compound 6 (1.87 g) in 15 mL of THF was cooled down to 0° C. in an ice bath and treated slowly with 1 M aqueous lithium hydroxide solution (3 mL). Reaction mixture was stirred at 0° C. for 3 hours, then warmed up to ambient temperature, treated with 3 mL of 1 M aqueous lithium hydroxide and diluted with 3 mL of methanol. The resulting mixture was stirred at room temperature for 3 hours until hydrolysis was complete (HPLC), then quenched by adding 1 M aqueous HCl solution to pH 7. Reaction mixture was then concentrated under reduced pressure and washed with 10 mL of MTBE. Aqueous layer was purified by reversed-phase chromatography (C18 column, 0-40% acetonitrile-water with 0.05% TFA). Pure product fractions were combined, concentrated under reduced pressure, and lyophilized to give compound 7 as a white powder (735 mg, 0.60 mmol, 60% yield over 2 steps). LRMS (ESI): m/z 1229.7 [M+H]⁺, Calcd for C₆₁H₉₆N₈O₁₈ m/z 1229.7.

### Preparation of (2S,3S,4S,5R,6S)-6-(5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((2S,5S,18R)-22-(2-((1,2-dimethylhydrazineyl)methyl)-1H-pyrrolo[2,3-b]pyridin-1-yl)-5-isopropyl-2-methyl-4,7,17,20-tetraoxo-18-(sulfomethyl)-10,13-dioxa-3,6,16,19-tetraazadocosanamido)phenoxy)-3,4,5-trihydroxytetrahydro-2H-pyran-2-carboxylic acid (8)

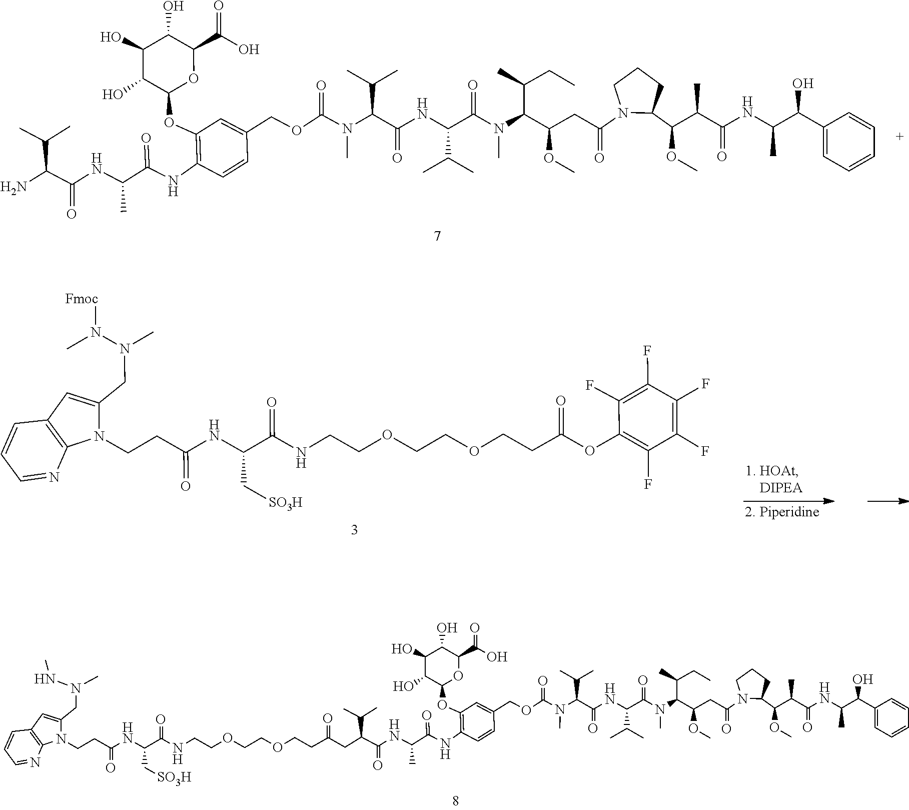

To a stirred solution of compound 7 (735 mg, 0.60 mmol) in 3 mL of anhydrous DMA were added DIPEA (0.21 mL, 1.2 mmol) and a solution PFP-ester 3 (575 mg, 0.60 mmol) in 2 mL of DMA at room temperature, followed by the addition of HOAt (84 mg, 0.60 mmol). The resulting mixture was stirred for 30 minutes until coupling was judged complete (HPLC analysis), then treated directly with 1.2 mL of piperidine at room temperature. After 15 minutes, reaction mixture was purified by reversed-phase chromatography (C18 column, 0-40% gradient of acetonitrile-water). Pure fractions were combined, concentrated under reduced pressure and room temperature, and lyophilized to give compound 8 (808 mg, 0.45 mmol, 75% yield) as a white fluffy powder. LRMS (ESI): m/z 1783.9 [M+H]⁺, Calcd for C₈₄H₁₃₀N₁₄O₂₆S m/z 1783.9.

### Synthesis of Belotecan Construct 21

Synthetic intermediates 4 and 9 were obtained commercially from Shanghai Medicilon and used as received. Belotecan 15 was purchased from AstaTech. All other reagents were obtained from commercial sources and used without purification.

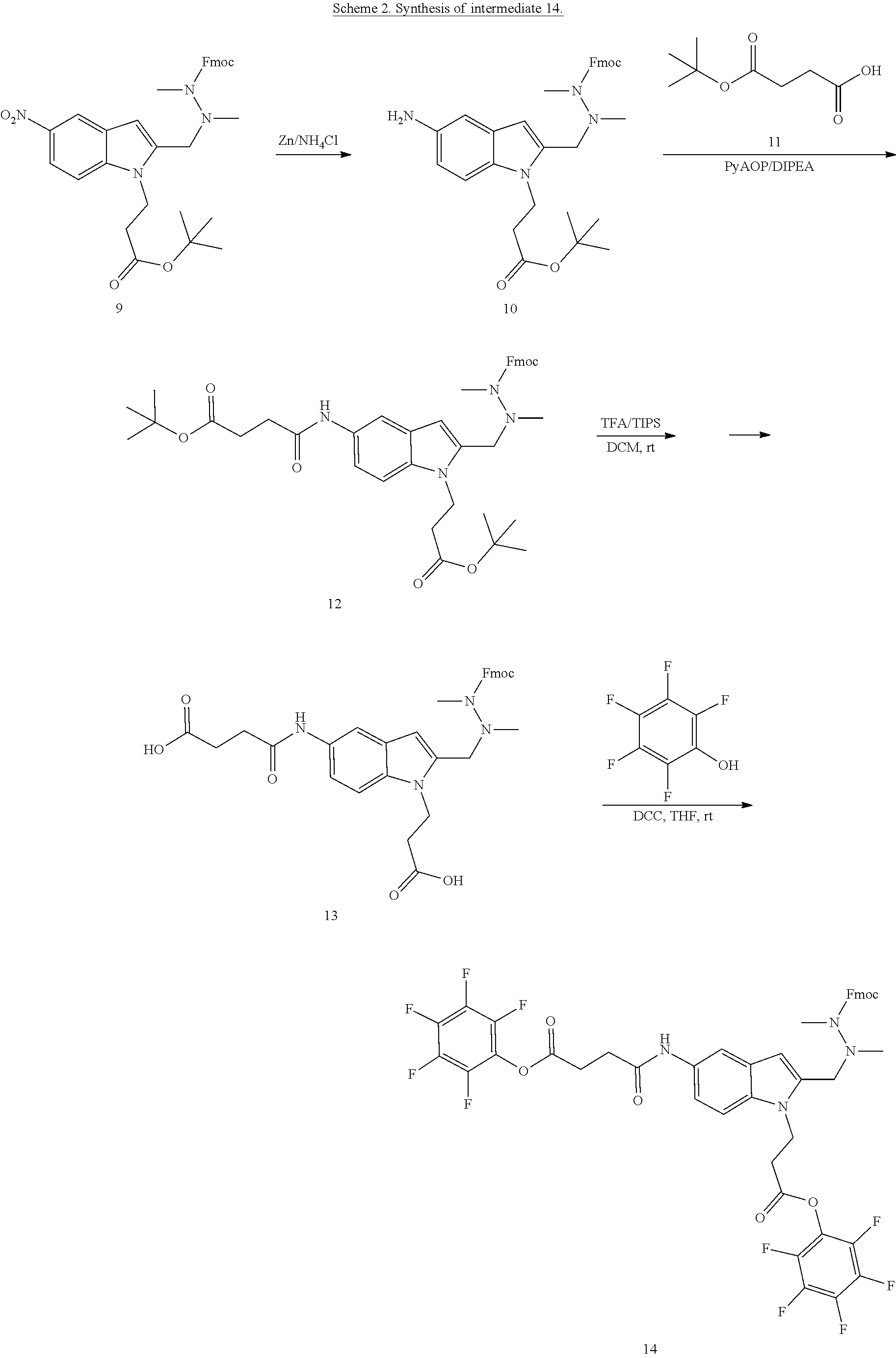

### Preparation of (9H-fluoren-9-yl)methyl 2-((5-amino-1-(3-(tert-butoxy)-3-oxopropyl)-1H-indol-2-yl)methyl)-1,2-dimethylhydrazine-1-carboxylate (10)

Nitro compound 9 (116 mg, 0.20 mmol) was dissolved in 1 mL of THF and combined with a solution of ammonium chloride (85 mg, 1.6 mmol) in 0.5 mL of water and 1 mL of methanol. The resulting mixture was vigorously stirred at room temperature and treated with zinc powder (104 mg, 1.6 mmol) in small portions over 5 minutes. Reaction mixture was stirred for 2 hours, solids were filtered off, filtrate was diluted with 20 mL of saturated aqueous ammonium chloride solution and extracted with ethyl acetate (2×25 mL). Organic extracts were dried over sodium sulfate, solvents removed under vacuum to give crude product 10 which was taken to the next step without purification. LRMS (ESI): m/z 555.3 [M+H]+, Calcd for C₃₃H₃₈N₄O₄ m/z 555.3.

### Preparation of (9H-fluoren-9-yl)methyl 2-((1-(3-(tert-butoxy)-3-oxopropyl)-5-(4-(tert-butoxy)-4-oxobutanamido)-1H-indol-2-yl)methyl)-1,2-dimethylhydrazine-1-carboxylate (12)

Crude compound 10 (˜0.20 mmol) was combined with 4-(tert-butoxy)-4-oxobutanoic acid 11 (40 mg, 0.23 mmol) in 2 mL of DMF. To this mixture were added DIPEA (0.12 mL, 0.6 mmol), followed by PyAOP (110 mg, 0.21 mmol) in one portion at room temperature. After 30 minutes, reaction was quenched by pouring into saturated aqueous ammonium chloride, extracted with ethyl acetate, washed with brine, dried over sodium sulfate. Solvent was removed under vacuum to give 120 mg (0.17 mmol, 85% yield over 2 steps) of product 12 as a dark oil which was used further without additional purification. LRMS (ESI): m/z 733.4 [M+Na]⁺, Calcd for C₄₁H₅₀N₄O₇ m/z 733.4.

### Preparation of 4-((2-((2-(((9H-fluoren-9-yl)methoxy)carbonyl)-1,2-dimethylhydrazinyl)methyl)-1-(2-carboxyethyl)-1H-indol-5-yl)amino)-4-oxobutanoic acid (13)

Bis-tert-butyl ester compound 12 (120 mg, 0.17 mmol) was dissolved in a mixture of 2 mL of anhydrous DCM, 2 mL of TFA, and 0.5 mL of trisopropylsilane. The resulting mixture was allowed to stand at room temperature for 4 hours. Solvents were removed under vacuum, and the residue was purified by reversed phase chromatography (C18 column, 0-70% v/v gradient of CH₃CN/H₂O with 0.05% TFA) to obtain 53 mg (0.09 mmol, 53% yield) of diacid product 13. LRMS (ESI): m/z 599.3 [M+H]+, Calcd for C₃₃H₃₄N₄O₇ m/z 599.2.

### Preparation of (9H-fluoren-9-yl)methyl 1,2-dimethyl-2-((1-(3-oxo-3-(perfluorophenoxy)propyl)-5-(4-oxo-4-(perfluorophenoxy)butanamido)-1H-indol-2-yl)methyl)hydrazine-1-carboxylate (14)

To a mixture of diacid 13 (50 mg, 0.084 mmol) and pentafluorophenol (46 mg, 0.25 mmol) in 2 mL of anhydrous THF were added DCC (51 mg, 0.25 mmol) in one portion at room temperature. The resulting mixture was stirred for 16 hours, solids were filtered off, filtrate concentrated, and purified by reversed phased chromatography (C18 column, 0-100% v/v gradient of CH₃CN/H₂O with 0.05% TFA). Fractions containing product were concentrated to about 20 mL, poured into 50 mL of 10% aqueous citric acid, and extracted with ethyl acetate (2×20 mL), dried over sodium sulfate. Solvents were removed under vacuum to give 67 mg (0.072 mmol, 86% yield) of bis-PFP ester product 14 as a dark viscous oil. LRMS (ESI): m/z 953.1 [M+Na]⁺, Calcd for C₄₅H₃₂F₁₀N₄O₇ m/z 953.2.

### Preparation of (2S,3S,4S,5R,6S)-6-(2-((S)-2-((S)-2-amino-3-methylbutanamido)propanamido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (16)

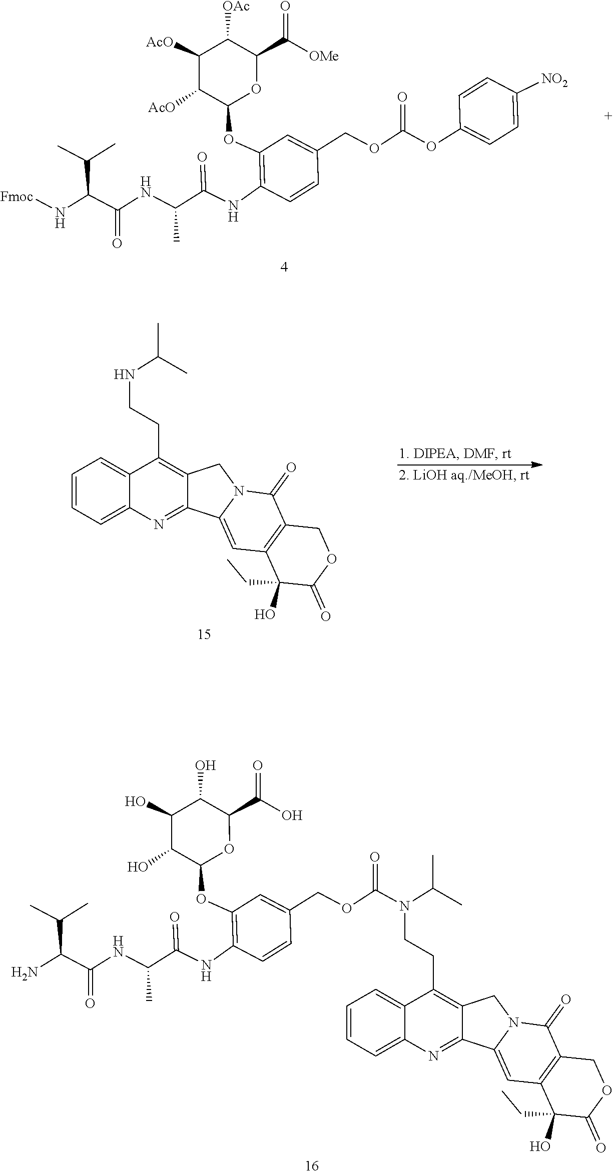

To a solution of belotecan 15 (HCl salt, 20 mg, 43 μmol) in 2 mL DMF were added 15 uL of DIPEA (86 μmol) and 6 mg of HOAt (43 μmol). The resulting mixture was treated with PNP carbonate 4 (43 mg, 43 μmol) at room temperature and stirred for one hour, then DMF was removed under vacuum. The residue was dissolved in 1 mL of MeOH and treated with 1 mL of 1M aqueous LiOH. After 10 minutes, 1 mL of 1M aqueous HCl was added to the mixture, followed by 1 mL of 0.5 M pH 4.7 acetate buffer. The resulting mixture was stirred for 30 minutes at room temperature and directly purified by reversed phase HPLC (C18 column, 0-50% v/v gradient of CH₃CN/H₂O with 0.05% TFA). Solvent was removed under vacuum to give 17 mg (18 μmol, 43% yield) of compound 16 as a glassy yellow solid. LRMS (ESI): m/z 945.4 [M+H]+, Calcd for C₄₇H₅₆N₆O₁₅ m/z 945.4.

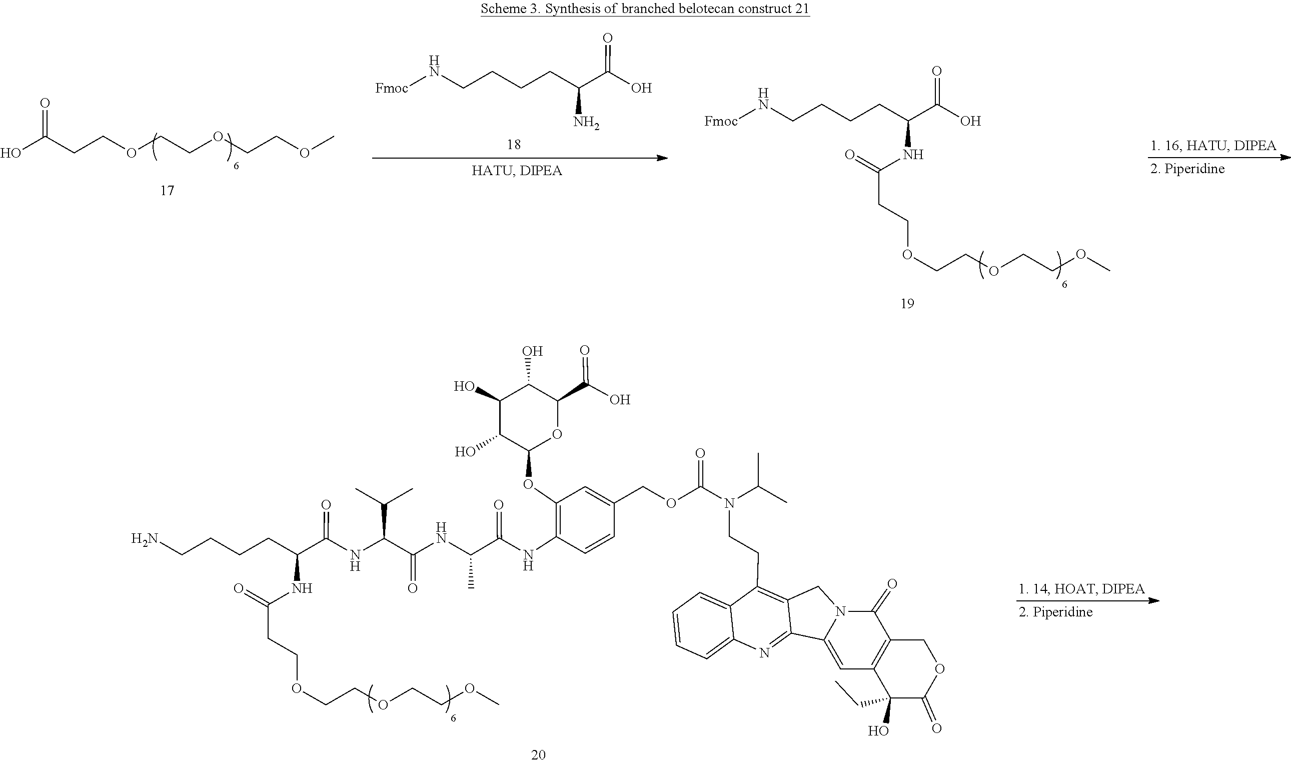

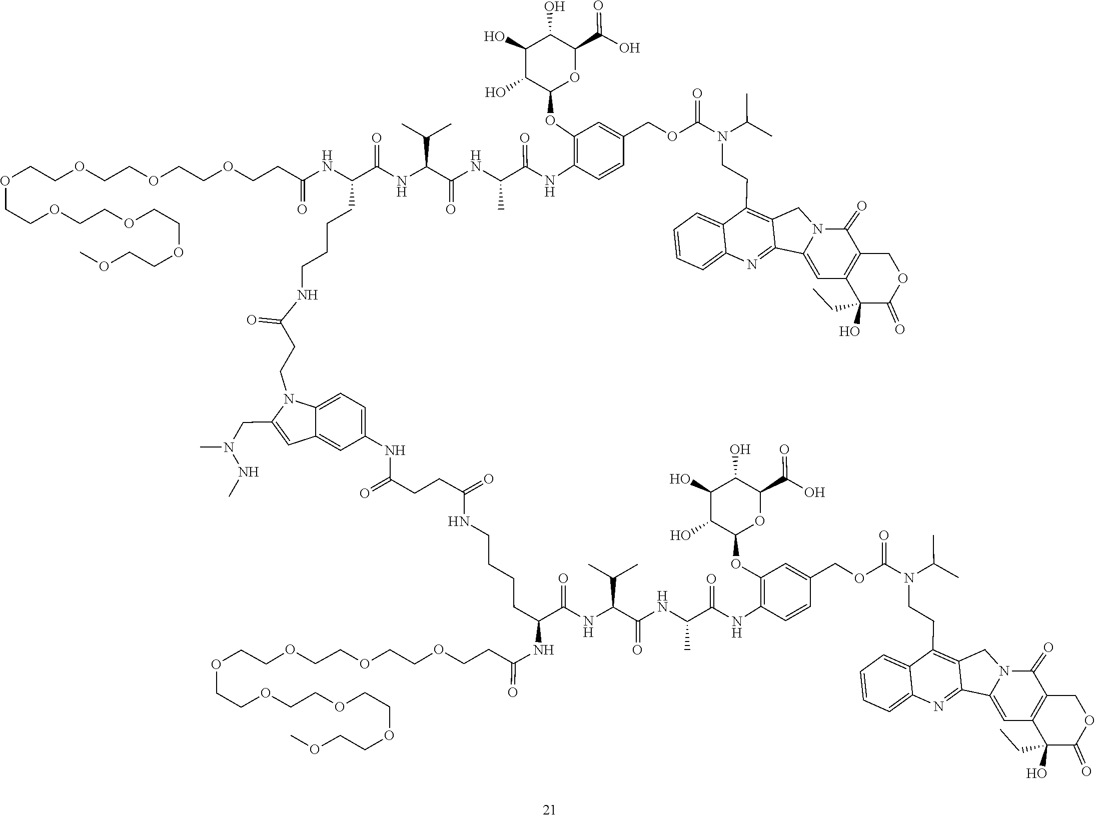 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

### Preparation of N⁶-(((9H-fluoren-9-yl)methoxy)carbonyl)-N²-(3-(2-(2-methoxyethoxy)ethoxy)propanoyl)-L-lysine 19

To a solution of mPEG8-acid 17 (100 mg, 0.24 mmol) in 2 mL of anhydrous DMF were added DIPEA (0.13 mL, 0.72 mmol) and HATU (93 mg, 0.24 mmol) at room temperature. The resulting mixture was stirred for one hour, then Lys(Fmoc)-OH 18 (89 mg, 0.24 mmol) was added to the mixture, and stirring continued for one hour. Reaction mixture was directly purified by reversed-phase chromatography HPLC (C18, 0-70% v/v MeCN—H₂O with 0.05% TFA) to give 120 mg of compound 19 (0.16 mmol, 67% yield) as a colorless oil. LRMS (ESI): m/z 763.4 [M+H]⁺, Calcd for C₃₉H₅₈N₂O₁₃ m/z 763.4.

### Preparation of (2S,3S,4S,5R,6S)-6-(2-((28S,31S,34S)-28-(4-aminobutyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (20)

To a solution of carboxylic acid 23 (45 mg, 59 μmol) in 3 mL of anhydrous DMF were added DIPEA (21 μL, 120 μmol) and HATU (22 mg, 59 μmol) at room temperature. The resulting mixture was stirred for 20 minutes and combined with amine 16 (55 mg, 58 μmol) in 1 mL of DMF. Reaction mixture was stirred for 30 minutes, then piperidine (115 μL, 1.2 mmol) was added to the mixture at room temperature. After 20 minutes, reaction mixture was directly purified by reversed-phase prep HPLC (C18, 0-50% v/v MeCN—H₂O with 0.05% TFA). Lyophilization of pure fractions afforded 34 mg (23 μmol, 40% yield) of compound 20 as a yellow powder. LRMS (ESI): m/z 1467.7 [M+H]+, Calcd for C₇₁H₁₀₂N₈O₂₅ m/z 1467.7.

### Preparation of (2S,3S,4S,5R,6S)-6-(2-((28S,31S,34S)-28-(4-(3-(5-((S)-28-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenyl)amino)-1-oxopropan-2-yl)amino)-3-methyl-1-oxobutan-2-yl)carbamoyl)-26,34-dioxo-2,5,8,11,14,17,20,23-octaoxa-27,33-diazaheptatriacontan-37-amido)-2-((1,2-dimethylhydrazineyl)methyl)-1H-indol-1-yl)propanamido)butyl)-31-isopropyl-34-methyl-26,29,32-trioxo-2,5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′:6,7]indolizino[1,2-b]quinolin-11-yl)ethyl)(isopropyl)carbamoyl)oxy)methyl)phenoxy)-3,4,5-trihydroxytetrahydro-2H-pyran-2-carboxylic acid (21)

To a mixture of compound 20 (34 mg, 23 μmol) and DIPEA (8 μL, 46 μmol) in 2 mL of DMA were added bis-PFP ester 14 (9.4 mg, 10.5 μmol), followed by HOAt (3 mg, 23 mol) at room temperature. The resulting mixture was allowed to stand for 30 minutes at room temperature, then piperidine (21 μL, 0.21 mmol) was added to the mixture at room temperature. After 20 minutes, reaction mixture was directly purified by reversed phase prep HPLC (C18, 0-50% v/v MeCN—H₂O with 0.05% TFA). Pure fractions were combined and lyophilized to afford compound 21 as a yellow solid (23 mg, 7 μmol, 67% yield). LRMS (ESI): m/z 1638.3 [M+H]²+, Calcd for C₁₆₀H₂₂₄N₂₀O₅₃ m/z 1638.8.

### Example 9

### Bioconjugation, Purification, and HPLC Analytics

HIPS conjugation of aldehyde-tagged antibodies Antibodies (15 mg/mL) bearing one aldehyde tag were conjugated to linker-payloads (see Example 7) at 1.1 mM, respectively. Reactions proceeded for 72 h at 37° C. in 20 mM sodium citrate, 50 mM NaCl pH 5.5 (20/50 buffer) containing 0.85-2.5% DMA. After conjugation, free drug was removed using a 30 kD MWCO 0.5 mL Amicon spin concentrator. Samples were added to the spin concentrator, centrifuged at 15,000×g for 7 min, then diluted with 450 μL 20 mM sodium citrate, 50 mM NaCl pH 5.5 and centrifuged again. The process was repeated 10 times. To determine the DAR of the final product, ADCs were examined by analytical chromatography using HIC (Tosoh #14947) or PLRP-RP (Agilent PL1912-1802 1000A, 8 um, 50×2.1 mm) columns. HIC analysis used mobile phase A: 1.5 M ammonium sulfate, 25 mM sodium phosphate pH 7.0, and mobile phase B: 25% isopropanol, 18.75 mM sodium phosphate pH 7.0. PLRP analysis used mobile phase A: 0.1% trifluoroacetic acid in water, and mobile phase B: 0.1% trifluoroacetic acid in acetonitrile. Prior to PLRP analysis, sample was denatured with the addition of 50 mM DTT, 4 M guanidine HCl (final concentrations) and heating at 37° C. for 30 min. To determine aggregation, samples were analyzed using analytical size exclusion chromatography (SEC; Tosoh #08541) with a mobile phase of 300 mM NaCl, 25 mM sodium phosphate pH 6.8 with 5% isopropanol.

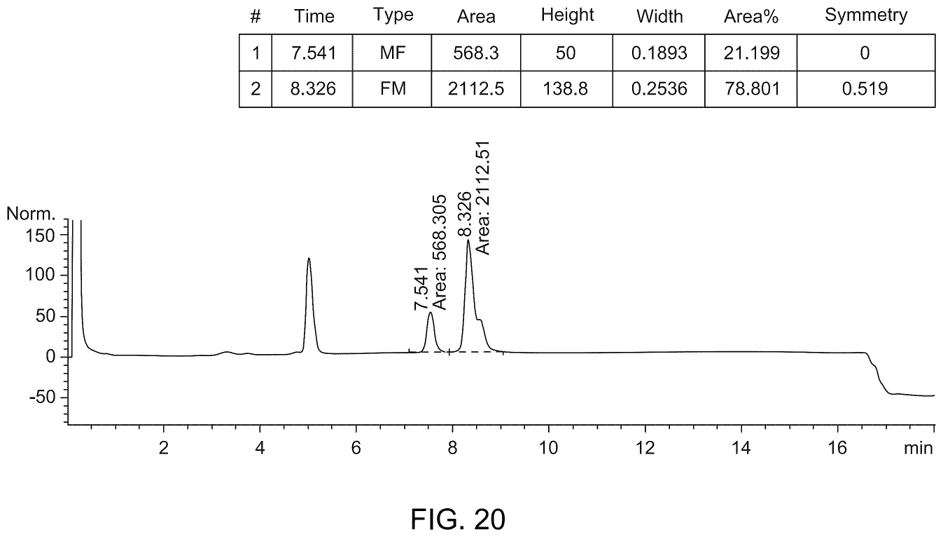

FIG. 20. Single-tagged CD30 VH4/VL4 antibody conjugated at 91N to Compound 8 (RED-601) yields a DAR of 1.58 as determined by PLRP.

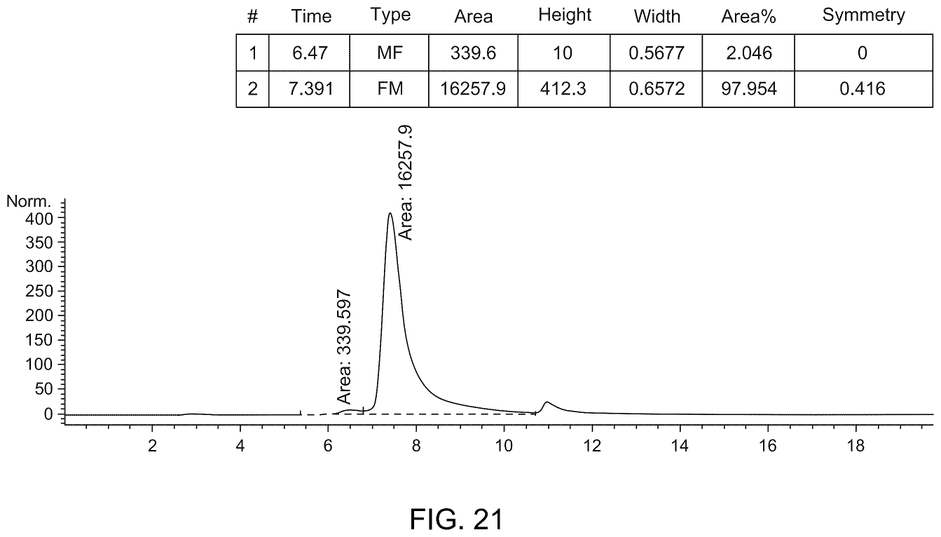

FIG. 21. Single-tagged CD30 VH4/VL4 antibody conjugated at 91N to Compound 8 (RED-601) is 98% monomeric as determined by SEC.

### Example 10: Xenograft Studies

L-82 Xenograft with CD30 ADCs

Female NOD/SCID mice were used (8 mice/group) for the study. Animals were inoculated subcutaneously in the flank with 10 million cells in 50% PBS/50% Matrigel. When tumors reached an average volume of 100 mm³, all animals were treated (Day 0) with a single 10 mg/kg intravenous dose of human IgG. Then, on Day 1, animals were treated with vehicle alone, with unconjugated antibody (3 mg/kg), or with an ADC at 1.5 or 3 mg/kg. The animals were monitored twice weekly for body weight and tumor size. Animals were euthanized when tumors reached 2000 mm³. ADCs were highly efficacious in this study.

FIG. 18 shows a graph of an L-82 xenograft study with a single intravenous dose of the listed anti-CD30 ADC on Day 0. VH4/VL4 Compound 8 (RED-601) uses the internal 91N tag and delivers half the payload dose as compared to Adcetris. At 50% ADC dosing (1.5 mg/kg) and equal dosing (3 mg/kg) VH4/VL4 Compound 8 was equally efficacious as compared with Adcetris, with all arms showing 8 complete responses out of 8 mice/group. The VH4/VL4 antibody alone had minimal activity.

Karpas 299 Xenograft with CD30 ADCs

Female CB17/SCID mice were used (6 mice/group) for the study. Animals were inoculated subcutaneously in the flank with 5 million cells in PBS. When tumors reached an average volume of 214 mm³, animals were treated with vehicle alone, with unconjugated antibody (3 mg/kg), or with an ADC at 1.5 or 3 mg/kg. The animals were monitored twice weekly for body weight and tumor size. Animals were euthanized when tumors reached 2000 mm³. ADCs were highly efficacious in this study.

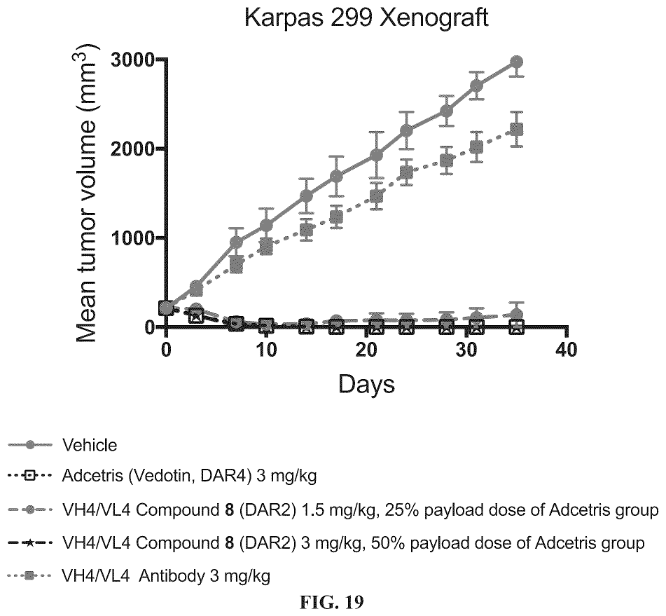

FIG. 19 shows a graph of a Karpas 299 xenograft study with a single intravenous dose of the listed anti-CD30 ADC on Day 0. VH4/VL4 Compound 8 (RED-601) uses the internal 91N tag and delivers half the payload dose as compared to Adcetris. At 50% ADC dosing (1.5 mg/kg) and equal dosing (3 mg/kg) VH4/VL4 Compound 8 gave 5/6 and 6/6 complete responses as compared with Adcetris, which gave 6/6 complete responses though with 2-fold the payload amount compared to VH4/VL4 Compound 8. The VH4/VL4 antibody alone had minimal activity.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A binding agent that specifically binds to CD30 protein, the binding agent comprising:
i) a variable heavy chain (VH) chain comprising a
sequence selected from:
(SEQ ID NO: 6)
QVQLQQSGPEVVKPGASVKVSCKASGYTFTDYYMTWVRQKPGQGLEW
MGWIYPGSGNTKYNQKFKGRVTITVDTSSSTAFMELSSLTSEDTAVYFC
ANYGNYWFAYWGQGTQVTVSA,
and
(SEQ ID NO: 11)
QVQLVQSGAEVKKPGASVKVSCKASGYTFTDYYITWVRQAPGQGLEWM
GWIYPGSGNTKYNEKFKGRVTITVDTSASTAYMELSSLRSEDTAVYYCA
NYGNYWFAYWGQGTLVTVSS;
and
ii) a variable light chain (VL) chain comprising
a sequence selected from:
(SEQ ID NO: 21)
DIVMTQSPASLAVSLGERATISCKSSQSVDFDGDSYLNWYQQKPGQPPK
LLIYAASTRESGVPARFSGSGSGTDFTLTISSLQEEDVATYYCQQSNED
PWTFGGGTKVEIK,
and
(SEQ ID NO: 26)
DIVLTQSPDSLAVSLGERATINCKASQSVDFDGDSYMNWYQQKPGQPPK
LLIYAASNRESGVPDRFSGSGSGTDFTLTISSLQAEDVAVYYCQQSNED
PWTFGGGTKVEIK.

2. The binding agent of claim 1, comprising a VH chain comprising SEQ ID NO: 11 and VL chain comprising SEQ ID NO: 26.

3. A binding agent that specifically binds to CD30, comprising:
VH chain comprising H-CDR1, H-CDR2, and H-CDR3 having the sequences of SEQ ID NOs: 31-33, respectively; and VL chain comprising L-CDR1, L-CDR2, and L-CDR3 having the sequences of SEQ ID NOs: 34-36, respectively; and wherein,
the VH chain comprises:
i) a heavy chain framework region 1 (HFR1) having the sequence of SEQ ID NO: 7, a heavy chain framework region 2 (HFR2) having the sequence of SEQ ID NO: 8, a heavy chain framework region 3 (HFR3) having the sequence of SEQ ID NO: 9, and a heavy chain framework region 4 (HFR4) having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and
the VL chain comprises:
i) a light chain framework region 1 (LFR1) having the sequence of SEQ ID NO: 22, a light chain framework region 2 (LFR2) having the sequence of SEQ ID NO: 23, a light chain framework region 3 (LFR3) having the sequence of SEQ ID NO: 24, and a light chain framework region 4 (LFR4) having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 30.

4. A binding agent that specifically binds to an antigen, comprising:
a VH chain comprising H-CDR1, H-CDR2, and H-CDR3 and a VL chain comprising L-CDR1, L-CDR2, and L-CDR3, wherein the complementarity determining regions determining the binding specificity of the binding agent for the antigen, and wherein, in the binding agent:
the VH chain comprises:
i) a HFR1 having the sequence of SEQ ID NO: 7, a HFR2 having the sequence of SEQ ID NO: 8, a HFR3 having the sequence of SEQ ID NO: 9, and a HFR4 having the sequence of SEQ ID NO: 10; or
ii) a HFR1 having the sequence of SEQ ID NO: 12, a HFR2 having the sequence of SEQ ID NO: 13, a HFR3 having the sequence of SEQ ID NO: 14, and a HFR4 having the sequence of SEQ ID NO: 15; and
the VL chain comprises:
i) a LFR1 having the sequence of SEQ ID NO: 22, a LFR2 having the sequence of SEQ ID NO: 23, a LFR3 having the sequence of SEQ ID NO: 24, and a LFR4 having the sequence of SEQ ID NO: 25; or
ii) a LFR1 having the sequence of SEQ ID NO: 27, a LFR2 having the sequence of SEQ ID NO: 28, a LFR3 having the sequence of SEQ ID NO: 29, and a LFR4 having the sequence of SEQ ID NO: 30.

5. The binding agent of claim 1, wherein the binding agent specifically binds to CD30, and comprises: a VH chain comprising H-CDR1, H-CDR2, and H-CDR3 having the sequences of SEQ ID NOs: 31-33, respectively; and VL chain comprising L-CDR1, L-CDR2, and L-CDR3 having the sequences of SEQ ID NOs: 34-36, respectively.

6. The binding agent of any one of claims 1-5, wherein the binding agent is a chimeric antibody.

7. The binding agent of any one of claims 1-6, wherein the binding agent is selected from the group consisting of: T-cell receptor, T-cell receptor like antibody, an IgG, Fv, single chain antibody, scFv, Fab, F(ab′)2, or Fab′.

8. The binding agent of any one of claims 1-7, wherein the binding agent is an IgG.

9. The binding agent of claim 8, wherein the IgG is an IgG1.

10. The binding agent of any one of claims 1-7, wherein the binding agent is a Fab.

11. The binding agent of any one of claims 1-7, wherein the binding agent is an scFv.

12. A bispecific binding agent comprising a first antigen-binding domain that specifically binds CD30, and wherein the first antigen binding domain comprises a VH chain and a VL chain as defined in any one of claims 1 to 5.

13. The binding agent of any one of claims 1-12, wherein the binding agent is detectably labeled.

14. The binding agent of any one of claims 1-12, wherein the binding agent is conjugated to an active agent.

15. The binding agent of claim 14, wherein the active agent is a cytotoxin.

16. The binding agent of any one of claims 1-15, wherein the binding agent comprises a constant region amino acid sequence comprising an amino acid sequence of a sulfatase motif.

17. The binding agent of any one of claims 1-16, wherein the binding agent is an antibody comprising a constant region amino acid sequence comprising an amino acid sequence of a sulfatase motif, and wherein the sulfatase motif is modified to comprise a 2-formylglycine (fGly) moiety.

18. The binding agent of claim 17, comprising the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the antibody, X¹ is present; and
X² and X³ are each independently any amino acid.

19. The binding agent of claim 18, wherein the sequence is L(fGly)TPSR.

20. The binding agent antibody of claim 18, wherein
Z³⁰ is selected from R, K, H, A, G, L, V, I, and P;
X¹ is selected from L, M, S, and V; and
X² and X³ are each independently selected from S, T, A, V, G, and C.

21. The binding agent of any one of claims 18 to 20, wherein the sequence is at a C-terminus of a heavy chain constant region of the antibody.

22. The binding agent of claim 21, wherein the heavy chain constant region comprises the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid,
wherein the sequence is C-terminal to the amino acid sequence SLSLSPG.

23. The binding agent of claim 21, wherein the heavy chain constant region comprises the sequence SPGSL(fGly)TPSRGS.

24. The binding agent of claim 21, wherein
Z³⁰ is selected from R, K, H, A, G, L, V, I, and P;
X¹ is selected from L, M, S, and V; and
X² and X³ are each independently selected from S, T, A, V, G, and C.

25. The binding agent of any one of claims 18 to 20, wherein the fGly moiety is positioned in a light chain constant region of the antibody.

26. The binding agent of claim 25, wherein the light chain constant region comprises the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the sequence KVDNAL, and/or is N-terminal to the sequence QSGNSQ.

27. The binding agent of claim 26, wherein the light chain constant region comprises the sequence KVDNAL(fGly)TPSRQSGNSQ.

28. The binding agent of claim 27, wherein
Z³⁰ is selected from R, K, H, A, G, L, V, I, and P;
X¹ is selected from L, M, S, and V; and
X² and X³ are each independently selected from S, T, A, V, G, and C.

29. The binding agent of any one of claims 18 to 20, wherein the fGly moiety is positioned in a heavy chain CH1 region of the antibody.

30. The binding agent of claim 29, wherein the heavy chain CH1 region comprises the sequence:
X¹(fGly)X²Z²⁰X³Z³⁰
wherein
Z²⁰ is either a proline or alanine residue;
Z³⁰ is a basic amino acid or an aliphatic amino acid;
X¹ may be present or absent and, when present, can be any amino acid, with the proviso that when the sequence is at the N-terminus of the conjugate, X¹ is present; and
X² and X³ are each independently any amino acid, and
wherein the sequence is C-terminal to the amino acid sequence SWNSGA and/or is N-terminal to the amino acid sequence GVHTFP.

31. The binding agent of claim 30, wherein the heavy chain CH1 region comprises the sequence SWNSGAL(fGly)TPSRGVHTFP.

32. The binding agent of claim 30, wherein
Z³⁰ is selected from R, K, H, A, G, L, V, I, and P;
X¹ is selected from L, M, S, and V; and
X² and X³ are each independently selected from S, T, A, V, G, and C.

33. The binding agent of any one of claims 29-30 and 32, wherein the binding agent comprises the sequence X¹(fGly)X²Z²⁰X³Z³⁰ before the asparagine residue at the 91^{st} position of the heavy chain CH1 region.

34. The binding agent of any one of claims 29-30 and 32-33, wherein the heavy chain CH1 region comprises the sequence of SEQ ID NO: 175 or a sequence having at least 90% sequence identity to the sequence of SEQ ID NO: 175.

35. The binding agent of claim 33 or 34, wherein the sequence X¹(fGly)X²Z²⁰X³Z³⁰ comprises the sequence LCTPSR (SEQ ID NO: 58).

36. The binding agent of claim 35, comprising the sequence LCTPSR (SEQ ID NO: 58) before the asparagine residue at the 91^{st} position of SEQ ID NO: 175 to produce an antibody comprising the sequence of KPSLCTPSRNTK (SEQ ID NO: 189).

37. The binding agent of claim 36, comprising the following sequence:
(SEQ ID NO: 190)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSLCTPSRNT
KVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFF
LYSKLTVDKSRWQQGNVFSCSVMHEALHNHYTQKSLSLSPGK.

38. The binding agent of any one of claims 18 to 20, wherein the fGly moiety is positioned in a heavy chain CH2 region of the antibody.

39. The binding agent of any one of claims 18 to 20, wherein the fGly moiety is positioned in a heavy chain CH3 region of the antibody.

40. The binding agent of any one of claims 17 to 39, wherein the binding agent comprises a heterologous moiety covalently linked to the antibody via the fGly moiety.

41. The binding agent of claim 40, wherein the heterologous moiety is a drug, oligonucleotide, protein, lipid nanoparticle, viral particle, a toxin, a detectable label, a water-soluble polymer, or a synthetic peptide.

42. A nucleic acid encoding a variable heavy (VH) chain, a variable light chain (VL), or both, of the binding agent of any one of claims 1 to 41.

43. The nucleic acid of claim 42, wherein the binding agent is a single chain antibody, and wherein the nucleic acid encodes the single chain antibody.

44. The nucleic acid of claim 43, wherein the single chain antibody is an scFv.

45. A recombinant expression vector comprising the nucleic acid of any one of claims 42 to 44, wherein the nucleic acid is operably linked to a transcriptional control element that is active in a eukaryotic cell.

46. A cell comprising the nucleic acid of any one of claims 42 to 44 or the expression vector of claim 45.

47. The cell of claim 42, wherein the nucleic acid encodes the VH chain and the VL chain of the binding agent.

48. The cell of claim 47, wherein the binding agent is a single chain antibody, and wherein the nucleic acid encodes the single chain antibody.

49. The cell of claim 4, wherein the single chain antibody is an scFv.

50. A cell comprising:
a first nucleic acid encoding a VH chain of a binding agent; and
a second nucleic acid encoding a VL chain of the binding agent,
wherein the VH chain and the VL chain produces the binding agent according to any of claims 1 to 41.

51. The cell of claim 50, comprising:
a first expression vector comprising the first nucleic acid; and
a second expression vector comprising the second nucleic acid.

52. A fusion protein, comprising:
a VH chain, a VL chain, or both, of the binding agent of any one of claims 1 to 41; fused to a heterologous amino acid sequence.

53. A conjugate, comprising:
the binding agent of any one of claims 1 to 41; and
an agent conjugated to the binding agent.

54. The conjugate of claim 53, wherein the agent is selected from the group consisting of: a half-life extending moiety, a labeling agent, and a drug.

55. The conjugate of claim 54, wherein the conjugate is of formula (I): 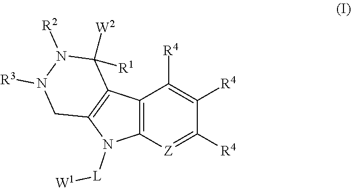
wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker;
W¹ is a drug; and
W² is the binding agent.

56. The conjugate of claim 55, wherein L comprises:
-(T¹-V¹)ₐ-(T²-V²)_{b}-(T³-V³)_{c}-(T⁴-V⁴)_{d}-(T⁵-V⁵)ₑ-(T⁶-V⁶)_{f}—,
wherein
a, b, c, d, e and f are each independently 0 or 1, wherein the sum of a, b, c, d, e and f is 1 to 6;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each m is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

57. The conjugate of claim 56, wherein:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₘ—, 4-amino-piperidine (4AP), MABO, MABC, PABO, PABC, PAB, PABA, PAP, PHP, an acetal group, a hydrazine, and an ester; and
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is 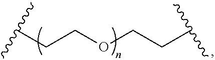
where n is an integer from 1 to 30;
EDA is an ethylene diamine moiety having the following structure: 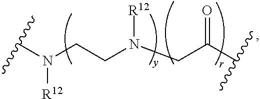
where y is an integer from 1 to 6 and r is 0 or 1;
4-amino-piperidine (4AP) is R¹²; and 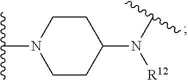
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

58. The conjugate of any of claims 56 to 57, wherein MABO, MABC, PABO, PABC, PAB, PABA, PAP and PHP are each optionally substituted with a glycoside.

59. The conjugate of claim 58, wherein the glycoside is selected from a glucuronide, a galactoside, a glucoside, a mannoside, a fucoside, O-GlcNAc, and O-GalNAc.

60. The conjugate of any of claims 56 to 59,
wherein:
T¹ is (C₁-C₁₂)alkyl and V¹ is —CO—;
T² is an amino acid analog and V² is —NH—;
T³ is (PEG)ₙ and V³ is —CO—;
T⁴ is AA and V⁴ is absent;
T⁵ is PABC and V⁵ is absent; and
f is 0.

61. The conjugate of any of claims 55 to 60, wherein the drug is monomethyl auristatin E (MMAE).

62. The conjugate of any one of claims 55 to 61, wherein the conjugate has the structure:

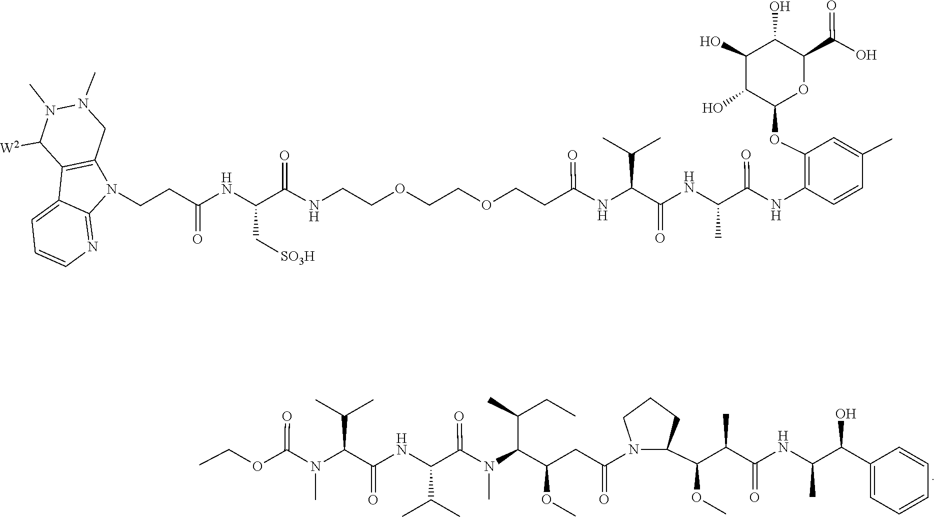

63. The conjugate of claim 54, wherein the conjugate is of formula (Ia): 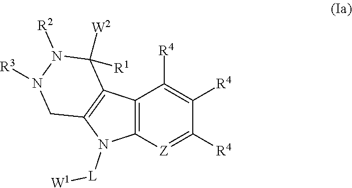
wherein
Z is CR⁴ or N;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is a linker comprising -(T¹-V¹)ₐ-(T²-V²)_{b}-(T³-V³)_{c}-(T⁴-V⁴)_{d}—, wherein a, b, c and d are each independently 0 or 1, where the sum of a, b, c and d is 1 to 4;
T¹, T², T³ and T⁴ are each independently selected from (C₁-C₁₂)alkyl, substituted (C₁-C₁₂)alkyl, (EDA)_{w}, (PEG)ₙ, (AA)ₚ, —(CR¹³OH)ₕ—, piperidin-4-amino (4AP), an acetal group, a hydrazine, a disulfide, and an ester, wherein EDA is an ethylene diamine moiety, PEG is a polyethylene glycol or a modified polyethylene glycol, and AA is an amino acid residue, wherein w is an integer from 1 to 20, n is an integer from 1 to 30, p is an integer from 1 to 20, and h is an integer from 1 to 12;
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
each R¹³ is independently selected from hydrogen, an alkyl, a substituted alkyl, an aryl, and a substituted aryl;
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
W¹ is a drug; and
W² is the binding agent.

64. The conjugate of claim 63, wherein:
T¹ is selected from a (C₁-C₁₂)alkyl and a substituted (C₁-C₁₂)alkyl;
T², T³ and T⁴ are each independently selected from (EDA)_{w}, (PEG)ₙ, (C₁-C₁₂)alkyl, substituted (C₁-C₁₂)alkyl, (AA)ₚ, —(CR¹³OH)ₕ—, 4-amino-piperidine (4AP), an acetal group, a hydrazine, and an ester; and
V¹, V², V³ and V⁴ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is 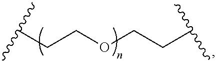
where n is an integer from 1 to 30;
EDA is an ethylene diamine moiety having the following structure: 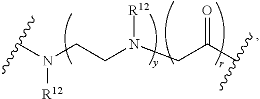
where y is an integer from 1 to 6 and r is 0 or 1;
4-amino-piperidine (4AP) is 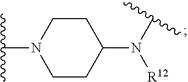
each R¹² and R¹⁵ is independently selected from hydrogen, an alkyl, a substituted alkyl, a polyethylene glycol moiety, an aryl and a substituted aryl, wherein any two adjacent R¹² groups may be cyclically linked to form a piperazinyl ring; and
R¹³ is selected from hydrogen, an alkyl, a substituted alkyl, an aryl, and a substituted aryl.

65. The conjugate of any one of claims 63 to 64, wherein T¹ is (C₁-C₁₂)alkyl, V¹ is —CO—, T² is 4AP, V² is —CO—, T³ is (C₁-C₁₂)alkyl, V³ is —CO—, T⁴ is absent and V⁴ is absent.

66. The conjugate of any one of claims 63 to 65, wherein the linker, L, comprises the following structure: 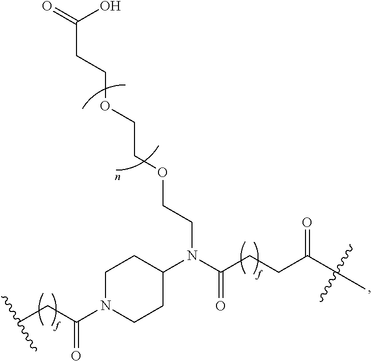
wherein
each f is independently an integer from 1 to 12; and
n is an integer from 1 to 30.

67. The conjugate of claim 54, wherein the conjugate is of formula (II): 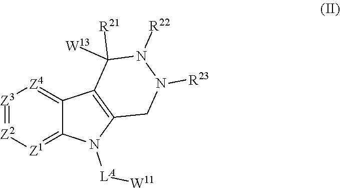
wherein:
Z¹, Z², Z³ and Z⁴ are each independently selected from CR²⁴, N and C-L^{B}-W¹², wherein at least one Z¹, Z², Z³ and Z⁴ is C-L^{B}-W¹²;
R²¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R²² and R²³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R²² and R²³ are optionally cyclically linked to form a 5 or 6-membered heterocyclyl;
each R²⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
W¹¹ is a first drug;
W¹² is a second drug; and
W¹³ is the binding agent.

68. The conjugate of claim 67, wherein Z¹ is CR²⁴.

69. The conjugate of claim 67, wherein Z¹ is N.

70. The conjugate of claim 67, wherein Z³ is C-L^{B}-W¹².

71. The conjugate of any of claims 67 to 70, wherein L^{A} comprises:
-(T¹-V¹)ₐ-(T²-V²)_{b}-(T³-V³)_{c}-(T⁴-V⁴)_{d}-(T⁵-V⁵)ₑ-(T⁶-V⁶)_{f}—,
wherein
a, b, c, d, e and f are each independently 0 or 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹—, —NR⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

72. The conjugate of claim 61, wherein MABO, MABC, PABO, PABC, PAB, PABA, PAP and PHP are each optionally substituted with a glycoside.

73. The conjugate of claim 72, wherein the glycoside is selected from a glucuronide, a galactoside, a glucoside, a mannoside, a fucoside, O-GlcNAc, and O-GalNAc.

74. The conjugate of any of claims 67 to 73,
wherein:
T¹ is (C₁-C₁₂)alkyl and V¹ is —CONH—;
T² is substituted (C₁-C₁₂)alkyl and V² is —CO—;
T³ is AA and V³ is absent;
T⁴ is PABC and V⁴ is absent; and
e and f are each 0.

75. The conjugate of any of claims 67 to 74, wherein L^{B} comprises:
-(T⁷-V⁷)_{g}-(T⁸-V⁸)ₕ-(T⁹-V⁹)-(T¹⁰-V¹⁰)ⱼ-(T¹¹-V¹¹)ₖ-(T¹²-V¹²)ᵢ-(T¹³-V¹³)ₘ,
wherein
g, h, i, j, k, l and m are each independently 0 or 1;
T⁷, T⁸, T⁹, T¹⁰, T⁴, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PHP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹—, —NR¹⁵CO, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

76. The conjugate of claim 71, wherein MABO, MABC, PABO, PABC, PAB, PABA, PAP and PHP are each optionally substituted with a glycoside.

77. The conjugate of any of claims 75 to 76, wherein the glycoside is selected from a glucuronide, a galactoside, a glucoside, a mannoside, a fucoside, O-GlcNAc, and O-GalNAc.

78. The conjugate of any of claims 75 to 77,
wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is AA and V¹⁰ is absent;
T¹¹ is PABC and V¹¹ is absent; and
l and m are each 0.

79. The conjugate of claim 67, wherein the conjugate has the structure:

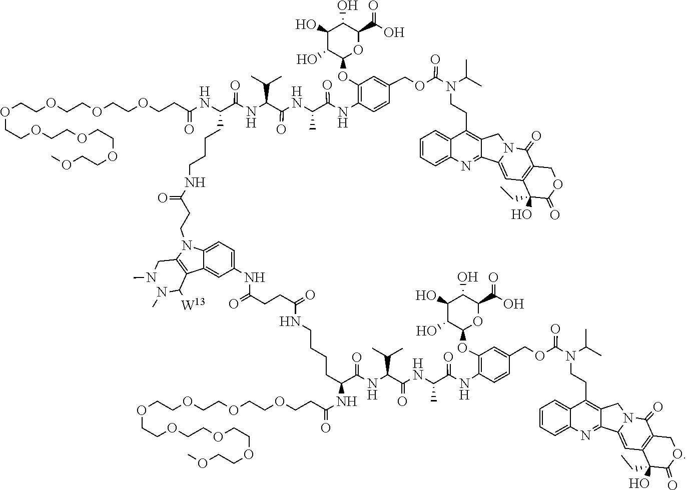

80. A pharmaceutical composition comprising:
a) the binding agent of any one of claims 1-41; and
b) a pharmaceutically acceptable carrier.

81. A pharmaceutical composition comprising:
a) the fusion protein of claim 52; and
b) a pharmaceutically acceptable carrier.

82. A pharmaceutical composition comprising:
a) the conjugate of any one of claims 53 to 79; and
b) a pharmaceutically acceptable carrier.

83. A method of treating a cell proliferative disorder in a subject, the method comprising:
administering to a subject having a cell proliferative disorder a therapeutically effective amount of the pharmaceutical composition of any one of claims 80 to 82.


SEQUENCE LISTING (195 sequences)

SEQ ID NO: 1 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 2 - protein, 25 aa, synthetic construct
QIQLQQSGPEVVKPGASVKISCKAS

SEQ ID NO: 3 - protein, 17 aa, synthetic construct
ITWVKQKPGQGLEWIGW

SEQ ID NO: 4 - protein, 38 aa, synthetic construct
KYNEKFKGKATLTVDTSSSTAFMQLSSLTSEDTAVYFC

SEQ ID NO: 5 - skipped

SEQ ID NO: 6 - protein, 117 aa, synthetic construct
QVQLQQSGPEVVKPGASVKVSCKASGYTFTDYYMTWVRQKPGQGLEWMGWIYPGSGNTKYNQKFKGRVTITVDTSSSTAFMELSSLTSEDTAVYFCANYGNYWFAYWGQGTQVTVSA

SEQ ID NO: 7 - protein, 25 aa, synthetic construct
QVQLQQSGPEVVKPGASVKVSCKAS

SEQ ID NO: 8 - protein, 17 aa, synthetic construct
MTWVRQKPGQGLEWMGW

SEQ ID NO: 9 - protein, 38 aa, synthetic construct
KYNQKFKGRVTITVDTSSSTAFMELSSLTSEDTAVYFC

SEQ ID NO: 10 - protein, 11 aa, synthetic construct
WGQGTQVTVSA

SEQ ID NO: 11 - protein, 117 aa, synthetic construct
QVQLVQSGAEVKKPGASVKVSCKASGYTFTDYYITWVRQAPGQGLEWMGWIYPGSGNTKYNEKFKGRVTITVDTSASTAYMELSSLRSEDTAVYYCANYGNYWFAYWGQGTLVTVSS

SEQ ID NO: 12 - protein, 25 aa, synthetic construct [sequence omitted]

SEQ ID NO: 13 - protein, 17 aa, synthetic construct
ITWVRQAPGQGLEWMGW

SEQ ID NO: 14 - protein, 38 aa, synthetic construct
KYNEKFKGRVTITVDTSASTAYMELSSLRSEDTAVYYC

SEQ ID NO: 15 - protein, 11 aa, synthetic construct
WGQGTLVTVSS

SEQ ID NO: 16 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 17 - protein, 26 aa, synthetic construct
DIVLTQSPASLAVSLGQRATISCKAS

SEQ ID NO: 18 - protein, 17 aa, synthetic construct
MNWYQQKPGQPPKVLIY

SEQ ID NO: 19 - protein, 34 aa, synthetic construct
ESGIPARFSGSGSGTDFTLNIHPVEEEDAATYYC

SEQ ID NO: 20 - protein, 10 aa, synthetic construct
FGGGTKLEIK

SEQ ID NO: 21 - protein, 111 aa, synthetic construct
DIVMTQSPASLAVSLGERATISCKSSQSVDFDGDSYLNWYQQKPGQPPKLLIYAASTRESGVPARFSGSGSGTDFTLTISSLQEEDVATYYCQQSNEDPWTFGGGTKVEIK

SEQ ID NO: 22 - protein, 26 aa, synthetic construct
DIVMTQSPASLAVSLGERATISCKSS

SEQ ID NO: 23 - protein, 17 aa, synthetic construct
LNWYQQKPGQPPKLLIY

SEQ ID NO: 24 - protein, 34 aa, synthetic construct
ESGVPARFSGSGSGTDFTLTISSLQEEDVATYYC

SEQ ID NO: 25 - protein, 10 aa, synthetic construct
FGGGTKVEIK

SEQ ID NO: 26 - protein, 111 aa, synthetic construct
DIVLTQSPDSLAVSLGERATINCKASQSVDFDGDSYMNWYQQKPGQPPKLLIYAASNRESGVPDRFSGSGSGTDFTLTISSLQAEDVAVYYCQQSNEDPWTFGGGTKVEIK

SEQ ID NO: 27 - protein, 26 aa, synthetic construct
DIVLTQSPDSLAVSLGERATINCKAS

SEQ ID NO: 28 - protein, 17 aa, synthetic construct
MNWYQQKPGQPPKLLIY

SEQ ID NO: 29 - protein, 36 aa, synthetic construct
NRESGVPDRFSGSGSGTDFTLTISSLQAEDVAVYYC

SEQ ID NO: 30 - skipped

SEQ ID NO: 31 - protein, 8 aa, synthetic construct
GYTFTDYY

SEQ ID NO: 32 - protein, 8 aa, synthetic construct
IYPGSGNT

SEQ ID NO: 33 - protein, 10 aa, synthetic construct
ANYGNYWFAY

SEQ ID NO: 34 - protein, 10 aa, synthetic construct
QSVDFDGDSY

SEQ ID NO: 35 - skipped

SEQ ID NO: 36 - protein, 9 aa, synthetic construct
QQSNEDPWT

SEQ ID NO: 37 - protein, 6 aa, synthetic construct
VCTPSR

SEQ ID NO: 38 - protein, 6 aa, synthetic construct
LCSPSR

SEQ ID NO: 39 - protein, 6 aa, synthetic construct
LCAPSR

SEQ ID NO: 40 - protein, 6 aa, synthetic construct
LCVPSR

SEQ ID NO: 41 - protein, 6 aa, synthetic construct
LCGPSR

SEQ ID NO: 42 - protein, 6 aa, synthetic construct
ICTPAR

SEQ ID NO: 43 - protein, 6 aa, synthetic construct
LCTPSK

SEQ ID NO: 44 - protein, 6 aa, synthetic construct
MCTPSK

SEQ ID NO: 45 - protein, 6 aa, synthetic construct
VCTPSK

SEQ ID NO: 46 - protein, 6 aa, synthetic construct
LCSPSK

SEQ ID NO: 47 - protein, 6 aa, synthetic construct
LCAPSK

SEQ ID NO: 48 - protein, 6 aa, synthetic construct
LCVPSK

SEQ ID NO: 49 - protein, 6 aa, synthetic construct
LCGPSK

SEQ ID NO: 50 - protein, 6 aa, synthetic construct
LCTPSA

SEQ ID NO: 51 - protein, 6 aa, synthetic construct
ICTPAA

SEQ ID NO: 52 - protein, 6 aa, synthetic construct
MCTPSA

SEQ ID NO: 53 - protein, 6 aa, synthetic construct
VCTPSA

SEQ ID NO: 54 - protein, 6 aa, synthetic construct
LCSPSA

SEQ ID NO: 55 - protein, 6 aa, synthetic construct
LCAPSA

SEQ ID NO: 56 - protein, 6 aa, synthetic construct
LCVPSA

SEQ ID NO: 57 - protein, 6 aa, synthetic construct
LCGPSA

SEQ ID NO: 58 - protein, 6 aa, synthetic construct
LCTPSR

SEQ ID NO: 59 - protein, 6 aa, synthetic construct
MCTPSR

SEQ ID NO: 60 - protein, 16 aa, synthetic construct
  MOD_RES 10: note = FORMYLATION
SLSLSPGSLGTPSRGS

SEQ ID NO: 61 - protein, 8 aa, synthetic construct
SLSLSPGK

SEQ ID NO: 62 - protein, 6 aa, synthetic construct
KVDNAL

SEQ ID NO: 63 - protein, 6 aa, synthetic construct
QSGNSQ

SEQ ID NO: 64 - protein, 17 aa, synthetic construct
  MOD_RES 7: note = FORMYLATION
KVDNALGTPSRQSGNSQ

SEQ ID NO: 65 - protein, 6 aa, synthetic construct
SWNSGA

SEQ ID NO: 66 - protein, 6 aa, synthetic construct
GVHTFP

SEQ ID NO: 67 - protein, 18 aa, synthetic construct
  MOD_RES 8: note = FORMYLATION
SWNSGALGTPSRGVHTFP

SEQ ID NO: 68 - protein, 6 aa, synthetic construct
ASTKGP

SEQ ID NO: 69 - protein, 7 aa, synthetic construct
KSTSGGT

SEQ ID NO: 70 - protein, 4 aa, synthetic construct
PEPV

SEQ ID NO: 71 - protein, 8 aa, synthetic construct
NSGALTSG

SEQ ID NO: 72 - protein, 21 aa, synthetic construct
NSGALTSGVHTFPAVLQSSGL

SEQ ID NO: 73 - protein, 5 aa, synthetic construct
QSSGL

SEQ ID NO: 74 - protein, 4 aa, synthetic construct
TQTY

SEQ ID NO: 75 - protein, 5 aa, synthetic construct
HKPSN

SEQ ID NO: 76 - protein, 22 aa, synthetic construct
EPKSCDKTHTCPPCPAPELLGG

SEQ ID NO: 77 - protein, 5 aa, synthetic construct
FPPKP

SEQ ID NO: 78 - protein, 5 aa, synthetic construct
ISRTP

SEQ ID NO: 79 - protein, 9 aa, synthetic construct
DVSHEDPEV

SEQ ID NO: 80 - protein, 7 aa, synthetic construct
SHEDPEV

SEQ ID NO: 81 - protein, 9 aa, synthetic construct
DGVEVHNAK

SEQ ID NO: 82 - protein, 5 aa, synthetic construct
QYNST

SEQ ID NO: 83 - protein, 5 aa, synthetic construct
VLTVL

SEQ ID NO: 84 - protein, 7 aa, synthetic construct
NKALPAP

SEQ ID NO: 85 - protein, 9 aa, synthetic construct
SKAKGQPRE

SEQ ID NO: 86 - protein, 7 aa, synthetic construct
KAKGQPR

SEQ ID NO: 87 - protein, 10 aa, synthetic construct
PPSRKELTKN

SEQ ID NO: 88 - protein, 5 aa, synthetic construct
YPSDI

SEQ ID NO: 89 - protein, 7 aa, synthetic construct
NGQPENN

SEQ ID NO: 90 - protein, 10 aa, synthetic construct
TPPVLDSDGS

SEQ ID NO: 91 - protein, 19 aa, synthetic construct
HEALHNHYTQKSLSLSPGK

SEQ ID NO: 92 - protein, 6 aa, synthetic construct
SLSPGK

SEQ ID NO: 93 - protein, 12 aa, synthetic construct
PCSRSTSESTAA

SEQ ID NO: 94 - protein, 5 aa, synthetic construct
FPEPV

SEQ ID NO: 95 - protein, 12 aa, synthetic construct
SGALTSGVHTFP

SEQ ID NO: 96 - protein, 6 aa, synthetic construct
QSSGLY

SEQ ID NO: 97 - protein, 5 aa, synthetic construct
VAGPS

SEQ ID NO: 98-100 - skipped (3 entries)

SEQ ID NO: 101 - protein, 5 aa, synthetic construct
VLTVV

SEQ ID NO: 102 - protein, 7 aa, synthetic construct
NKGLPAP

SEQ ID NO: 103 - protein, 9 aa, synthetic construct
SKTKGQPRE

SEQ ID NO: 104 - protein, 5 aa, synthetic construct
MTKNQ

SEQ ID NO: 105-106 - skipped (2 entries)

SEQ ID NO: 107 - protein, 10 aa, synthetic construct
TPPMLDSDGS

SEQ ID NO: 108 - protein, 4 aa, synthetic construct
GNVF

SEQ ID NO: 109 - skipped

SEQ ID NO: 110 - protein, 10 aa, synthetic construct
PCSRSTSGGT

SEQ ID NO: 111 - skipped

SEQ ID NO: 112 - protein, 19 aa, synthetic construct
SGALTSGVHTFPAVLQSSG

SEQ ID NO: 113 - skipped

SEQ ID NO: 114 - protein, 10 aa, synthetic construct
RVELKTPLGD

SEQ ID NO: 115 - protein, 7 aa, synthetic construct
CPRCPKP

SEQ ID NO: 116 - protein, 21 aa, synthetic construct
PKSCDTPPPCPRCPAPELLGG

SEQ ID NO: 117-121 - skipped (5 entries)

SEQ ID NO: 122 - protein, 10 aa, synthetic construct
PPSREEMTKN

SEQ ID NO: 123 - skipped

SEQ ID NO: 124 - protein, 8 aa, synthetic construct
SSGQPENN

SEQ ID NO: 125 - skipped

SEQ ID NO: 126 - protein, 7 aa, synthetic construct
HEALHNR

SEQ ID NO: 127 - skipped

SEQ ID NO: 128 - protein, 5 aa, synthetic construct
STKGP

SEQ ID NO: 129-132 - skipped (4 entries)

SEQ ID NO: 133 - protein, 11 aa, synthetic construct
CPAPEFLGGPS

SEQ ID NO: 134 - skipped

SEQ ID NO: 135 - protein, 9 aa, synthetic construct
DVSQEDPEV

SEQ ID NO: 136 - skipped

SEQ ID NO: 137 - protein, 7 aa, synthetic construct
NKGLPSS

SEQ ID NO: 138 - protein, 10 aa, synthetic construct
SKAKGQPREP

SEQ ID NO: 139 - protein, 10 aa, synthetic construct
PPSQEEMTKN

SEQ ID NO: 140-142 - skipped (3 entries)

SEQ ID NO: 143 - protein, 19 aa, synthetic construct
HEALHNHYTQKSLSLSLGK

SEQ ID NO: 144 - protein, 13 aa, synthetic construct
ASPTSPKVFPLSL

SEQ ID NO: 145 - protein, 5 aa, synthetic construct
QPDGN

SEQ ID NO: 146 - protein, 10 aa, synthetic construct
VQGFFPQEPL

SEQ ID NO: 147 - protein, 11 aa, synthetic construct
SGQGVTARNFP

SEQ ID NO: 148 - protein, 7 aa, synthetic construct
SGDLYTT

SEQ ID NO: 149 - protein, 4 aa, synthetic construct
PATQ

SEQ ID NO: 150 - protein, 4 aa, synthetic construct
HRPA

SEQ ID NO: 151 - protein, 5 aa, synthetic construct
LLGSE

SEQ ID NO: 152 - protein, 8 aa, synthetic construct
GLRDASGV

SEQ ID NO: 153 - protein, 10 aa, synthetic construct
SSGKSAVQGP

SEQ ID NO: 154 - protein, 4 aa, synthetic construct
GCYS

SEQ ID NO: 155 - protein, 4 aa, synthetic construct
CAEP

SEQ ID NO: 156 - protein, 24 aa, synthetic construct
SGNTFRPEVHLLPPPSEELALNEL

SEQ ID NO: 157 - protein, 5 aa, synthetic construct
ARGFS

SEQ ID NO: 158 - protein, 11 aa, synthetic construct
QGSQELPREKY

SEQ ID NO: 159 - protein, 4 aa, synthetic construct
AAED

SEQ ID NO: 160 - protein, 4 aa, synthetic construct
HEAL

SEQ ID NO: 161 - protein, 25 aa, synthetic construct
IDRLAGKPTHVNVSVVMAEVDGTCY

SEQ ID NO: 162 - protein, 11 aa, synthetic construct
NSGALCTPSRG

SEQ ID NO: 163 - protein, 9 aa, synthetic construct
NLCTPSRAP

SEQ ID NO: 164 - skipped

SEQ ID NO: 165 - protein, 6 aa, synthetic construct
RTVAAP

SEQ ID NO: 166 - protein, 5 aa, synthetic construct
YPREA

SEQ ID NO: 167 - protein, 8 aa, synthetic construct
DNALQSGN

SEQ ID NO: 168 - protein, 9 aa, synthetic construct
TEQDSKDST

SEQ ID NO: 169 - protein, 6 aa, synthetic construct
HQGLSS

SEQ ID NO: 170 - protein, 4 aa, synthetic construct
RGEC

SEQ ID NO: 171 - protein, 6 aa, synthetic construct
QPKAAP

SEQ ID NO: 172 - protein, 7 aa, synthetic construct
DFYPGAV

SEQ ID NO: 173 - protein, 8 aa, synthetic construct
DSSPVKAG

SEQ ID NO: 174 - protein, 6 aa, synthetic construct
APTECS

SEQ ID NO: 175 - protein, 99 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVE

SEQ ID NO: 176 - protein, 18 aa, synthetic construct
KVDNALLCTPSRQSGNSQ

SEQ ID NO: 177 - protein, 5 aa, synthetic construct
HHHHH

SEQ ID NO: 178 - protein, 6 aa, synthetic construct
HHHHHH

SEQ ID NO: 179 - protein, 10 aa, synthetic construct
EQKLISEEDL

SEQ ID NO: 180 - protein, 8 aa, synthetic construct
DYKDDDDK

SEQ ID NO: 181 - protein, 8 aa, synthetic construct
WSHPQFEK

SEQ ID NO: 182 - protein, 9 aa, synthetic construct
YPYDVPDYA

SEQ ID NO: 183 - protein, 5 aa, synthetic construct
RYIRS

SEQ ID NO: 184 - protein, 4 aa, synthetic construct
FHHT

SEQ ID NO: 185 - protein, 17 aa, synthetic construct
WEAAAREACCRECCARA

SEQ ID NO: 186 - protein, 5 aa, synthetic construct
GSGGS

SEQ ID NO: 187 - protein, 4 aa, synthetic construct
GGGS

SEQ ID NO: 188 - protein, 10 aa, synthetic construct
QKSLSLSPGK

SEQ ID NO: 189 - protein, 12 aa, synthetic construct
KPSLCTPSRNTK

SEQ ID NO: 190 - protein, 336 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSLCTPSRNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 10 aa, synthetic construct
GGGGGGGGGG

SEQ ID NO: 192 - protein, 10 aa, synthetic construct
KAKGLCTPSR

SEQ ID NO: 193 - protein, 8 aa, synthetic construct
GALTSGVH

SEQ ID NO: 194 - protein, 11 aa, synthetic construct
GALCTPSRGVH

SEQ ID NO: 195 - protein, 9 aa, synthetic construct
SLCTPSRGS